(19) United States
(12) Patent Application Publication
(10) Pub. No.: US 2009/0203547 A1
(43) Pub. Date: Aug. 13, 2009

(54) Gene and Cognate Protein Profiles and Methods to Determine Connective Tissue Markers in Normal and Pathologic Conditions

(76) Inventors: Albert Banes, Hillsborough, NC (US); Jie Qi, Chapel Hill, NC (US); Donald K. Bynum, Durham, NC (US); Beverly Koller, Chapel Hill, NC (US); Jeffrey Thompson, Durham, NC (US); Ann Fox, Broomfield, CO (US); Allison Nation, Victoria (AU)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 11/884,496

(22) PCT Filed: Feb. 21, 2006
(86) PCT No.: PCT/US2006/005948

§ 371 Date: Mar. 2, 2009

Related U.S. Application Data
(60) Provisional application No. 60/654,232, filed on Feb. 18, 2005.

(51) Int. Cl. C40B 40/06; C12Q 1/68

(52) U.S. Cl. 506/16; 435/6

(57) ABSTRACT

Differences in gene expression between connective tissue cells (e.g., tendon cells) and other closely related cell types are disclosed. Also disclosed are expression profiles between tendon cells under different genetic and environmental influences. The presently disclosed expression profiles are useful as diagnostic markers as well as markers that can be used to monitor disease states, disease progression, injury repair, drug toxicity, drug efficacy, and drug metabolism.

### RELATED APPLICATIONS

This application claims the benefit of U.S. Provisional Patent Application Ser. No. 60/654,232, filed Feb. 18, 2005, the disclosure of which is incorporated herein by reference in its entirety.

### TECHNICAL FIELD

The presently disclosed subject matter identifies differences in gene expression between cells and other closely related cell types. For example, gene expression in tendon cells relative to muscle cells is examined. The presently disclosed subject matter also identifies expression profiles between cells under different genetic and environmental influences. The presently disclosed subject matter also identifies expression profiles that serve as useful diagnostic markers as well as markers that can be used to monitor disease states, disease progression, injury repair, drug toxicity, drug efficacy, and drug metabolism.

### SEQUENCE LISTING PROVIDED ON CD-R

The Sequence Listing associated with the instant disclosure has been submitted as a 2.4 MB file on CD-R (in triplicate) instead of on paper. Each CD-R is marked in indelible ink to identify the Applicants, Title, File Name (421-140 PCT.ST25.txt)), Creation Date (Feb. 21, 2006), Computer System (IBM-PC/MS-DOS/MS-Windows), and Docket No. (421-140 PCT). The Sequence Listing submitted on CD-R is hereby incorporated by reference into the instant disclosure.

### BACKGROUND

A goal of the fields of genomics and proteomics is to utilize expression profiles of tissues to establish molecular markers that describe a given tissue at a stage of phenotype development from neonatal to juvenile to mature. In addition, a goal of these disciplines and technologies is to discover molecular markers that can be used to diagnose a stage of pathology. In some cases, an early stage of development might share some markers with a stage of pathology as in early markers of development recurring during healing from a wound. In other cases, a novel marker might be present that is indicative of a stage of disease such as a specific cancer such as breast or prostate cancer.

In the case of marker selection for connective tissues such as tendon, little work has been done to develop methodologies with respect to the selection of markers or to the development of expression profiles that are specific to such tissues. The identification of specific markers and the elucidation of changes in gene expression profiles that occur during injury and/or disease processes, as well as during the repair of and/or recovery from the same, would be extremely valuable for the diagnosis and/or monitoring of connective tissue disorders.

### SUMMARY

The presently disclosed subject matter provides methods for detecting connective tissue-specific gene expression in a sample. In some embodiments, the methods comprise detecting a level of expression in a sample of at least one gene for which expression is connective tissue-specific. In some embodiments, the connective tissue is selected from the group consisting of muscle and tendon. In some embodiments, the connective tissue is tendon. In some embodiments, the at least one gene is selected from the group consisting of those genes listed in Tables 1-4. In some embodiments, the detecting comprising hybridizing a nucleic acid isolated from the sample to an array comprising the at least one gene.

The presently disclosed subject matter also provides methods for diagnosing a disease of or an injury to a connective tissue in a mammalian subject. In some embodiments, the methods comprise detecting a level of expression in a biological sample of at least one gene for which an expression level is indicative of disease or injury in a connective tissue. In some embodiments, the connective tissue is selected from the group consisting of muscle and tendon. In some embodiments, the connective tissue is tendon. In some embodiments, the at least one gene is selected from the group consisting of those genes listed in Tables 1-4. In some embodiments, differential expression of at least one of the genes listed in Tables 1-4 is indicative of a disease or injury to a tendon. In some embodiments, the detecting comprising hybridizing a nucleic acid isolated from a sample isolated from the mammalian subject to an array comprising the at least one gene.

The presently disclosed subject matter also provides methods for detecting the progression of a disease of or an injury to a connective tissue in a mammalian subject. In some embodiments, the methods comprise detecting a level of expression in a biological sample of at least one gene for which an expression level is indicative of progression of a disease or injury in a connective tissue. In some embodiments, the connective tissue is selected from the group consisting of muscle and tendon. In some embodiments, the connective tissue is tendon. In some embodiments, the at least one gene is selected from the group consisting of those genes listed in Tables 1-4. In some embodiments, differential expression of at least one of the genes listed in Tables 1-4 is indicative of progression of a disease of or an injury to a tendon. In some embodiments, the detecting comprising hybridizing a nucleic acid isolated from a sample isolated from the mammalian subject to an array comprising the at least one gene.

The presently disclosed subject matter also provides methods for monitoring the treatment of a mammalian subject with a disease of or an injury to a connective tissue. In some embodiments, the methods comprise (a) providing a treatment to the subject; (b) detecting a level of expression of at least one gene from a cell or biological sample from the subject; and (c) comparing the level of expression detected in step (b) to a level of expression from a cell population comprising normal connective tissue cells, to a level of expression from a cell population comprising diseased or injured connective tissue, or both. In some embodiments, the connective tissue is selected from the group consisting of muscle and tendon. In some embodiments, the connective tissue is tendon. In some embodiments, the at least one gene is selected from the group consisting of those genes listed in Tables 1-4. In some embodiments, differential expression of at least one of the genes listed in Tables 1-4 is indicative of an effect of the treatment provided on a disease of or an injury to a tendon. In some embodiments, the detecting comprising hybridizing a nucleic acid isolated from a sample isolated from the mammalian subject to an array comprising the at least one gene.

The presently disclosed subject matter also provides kits for detecting expression of a gene differentially expressed in a connective tissue. In some embodiments, the kits comprise a plurality of reagents that can be used to detect expression levels for at least one gene for which expression is connective tissue-specific. In some embodiments, the at least one gene is selected from the group consisting of those genes listed in Tables 1-4. In some embodiments, the plurality of reagents comprise at least one oligonucleotide pair that can be used to specifically amplify at least one of the genes listed in Tables 1-4. In some embodiments, the kits further comprise one or more solid supports comprising one or more oligonucleotides attached thereto that specifically bind to at least one of the genes listed in Tables 1-4. In some embodiments, the one or more solid supports comprise an array, a microarray, or combinations thereof.

Accordingly, it is an object of the presently disclosed subject matter to provide specific marker genes and profiles of gene expression changes that occur as a result of, and subsequent to, connective tissue injury and/or disease. This and other objects are achieved in whole or in part by the presently disclosed subject matter.

An object of the presently disclosed subject matter having been stated above, other objects and advantages of the presently disclosed subject matter will become apparent to those of ordinary skill in the art after a study of the following description and non-limiting Examples.

### BRIEF DESCRIPTION OF THE SEQUENCE LISTING

SEQ ID NOs: 1-724 correspond to publicaly available nucleotide sequences for the database Accession Numbers presented in Tables 1-4.

### DETAILED DESCRIPTION

A goal in the connective tissue field, including that of hard tissues (bone, cartilage, fibrocartilage) as well as soft connective tissues (tendons, ligaments, menisci, muscle, fascia, sheaths, etc.) is to develop specific markers that characterize a given tissue, particularly with respect to pathology and staging of disease and/or injury processes. Investigators generally focus on the study of naturally occurring diseases to search for pathognomonic markers for cells and/or tissues of interest based on the assumption that one can learn about normal tissue development from studying pathologic processes. Important areas in hard tissue biology include rheumatoid arthritis and the search for markers that indicate a stage of the disease and whether or not it is progressing, is static, or is regressing.

The practical importance of finding and utilizing such markers and assessment strategies includes the ability to perform drug discovery research to identify pharmaceutical therapies that block or modulate the disease and to stage the disease to discern if the treatment therapy is working. Other practical outcomes of the latter diagnostic test data include, but are not limited to allowing judgments to be made as to whether a patient should receive a given treatment, whether insurers should pay for the treatment, and whether or not a patient is responding to the treatment and should continue a given drug therapy.

During the past decade, advances in the technology of disease markers has drastically changed from randomly searching for molecules that are affected by disease to those which are specifically regulated or co-regulated differently in disease versus non-disease states and represent an expression profile of the disease state. In addition, the use of gene arrays wherein an investigator can sample the expression profile of an entire transcriptome at any point in time has allowed the development of focused strategies to select environmental conditions that favor the specific marker discovery.

One form of a gene array is a representation of a portion of each gene expressed by mammalian cells as an oligonucleotide chemically immobilized to a glass surface in a “spot”. Each spot is about 10 microns in diameter in a specific location on a glass slide that is 25×75 mm in dimension. In this way, a representation of at least 40,000 genes as oligonucleotide arrays can be positioned on the glass surface. One can then isolate RNA (total ribonucleic acid, although the important part of the sample is the messenger RNA (mRNA)) from a tissue specimen, convert the RNA into cDNA (complementary deoxyribonucleic acid), prepare fluorescently labeled (green dye, Cy 3) control cDNA from one specimen and fluorescently labeled (red dye, Cy 5) test cDNA from a subject, then hybridize the two differently colored cDNAs to the oligonucleotide array on the glass slide in a special hybridization chamber. Once the excess colored sample cDNAs are washed from the slide, the array can be visualized as colored spots. A spot representing a specific oligonucleotide and therefore a specific gene product that is colored green is one that is more highly expressed in the control specimen than in the test specimen. Likewise, a spot that is more highly colored red is one that is expressed more highly in the test specimen than in the control specimen.

In this way, one can compare the relative expression levels of each gene represented by an oligonucleotide in the gene array. There are programs that allow the analysis of the fluorescence intensity of each dye for each sample at each spot. The program allows for the accurate quantitation of the fluorescence intensities for each candidate cDNA as well as a comparison between the two specimens on each slide. The latter example is of a direct comparison between samples. One can also make an indirect comparison between and among samples hybridized to targets on other slides, as long as the slides are of high quality and reproducibility. One such slide type is that produced by Agilent Technologies, Inc. (Palo Alto, Calif., United States of America), and is the 44 k whole mouse genome or the whole human genome slide. The spot intensities can be read in a slide reader, specially designed to read this type of slide to yield intensities for each spot. Quality control of control spots that are distributed over the slide is also done. Once this basic spot intensity quantitation is performed, then intensities of replicate spots can be determined among three or more replicates of each sample on different slides.

A further technique that is used to analyze the reproducibility of the expression levels of each spot is a statistical measure of the mean and standard deviation. A SAM (supervised analysis of microarray; Tusher et al., 2001) plot can then be calculated which yields the number of genes whose expression levels are statistically different between the two samples. SAGE analysis (supervised analysis of gene arrays) includes partitioning the data into groups of genes that are expressed by 2, 3, 4, 8, and more fold differences, usually in two fold increments. The data are generally expressed as log base 2 of the mean of the fluorescence intensities for each spot. In this way, one can select genes that are highly overexpressed or underexpressed in any comparison.

### I. DEFINITIONS

Unless otherwise defined, all technical and scientific terms used herein have the same meaning as commonly understood by one of ordinary skill in the art to which the presently disclosed subject matter pertains. For clarity of the present specification, certain definitions are presented hereinbelow.

Following long-standing patent law convention, the articles “a”, “an”, and “the” refer to “one or more” when used in this application, including in the claims. For example, the phrase “a tendon cell” refers to one or more tendon cells. Similarly, the phrase “at least one”, when employed herein to refer to an oligonucleotide, a gene, or any other entity, refers to, for example, 1, 2, 3, 4, 5, 6, 7, 8, 9, 10, 15, 20, 25, 50, 100, or more of that entity. Thus, the phrase “at least one gene” used in the context of the genes disclosed in Tables 1-4, refers to 1, 2, 3, 4, 5, 6, 7, 8, 9, 10, up to every gene disclosed in Tables 1-4, including every value in between.

As used herein, the phrase “biological sample” refers to a sample isolated from a subject (e.g., a biopsy) or from a cell or tissue from a subject (e.g., RNA isolated from, or cDNA reverse transcribed and/or derived therefrom). In some embodiments, a biological sample is a clinical sample such as a biopsy or a sample otherwise removed from a subject for any purpose. Biological samples can be of any biological tissue or fluid or cells from any organism as well as cells cultured in vitro, such as cell lines and tissue culture cells. Frequently the sample will be a “clinical sample” which is a sample derived from a patient (i.e., a subject undergoing a diagnostic procedure and/or a treatment). Typical clinical samples include, but are not limited to, blood, blood cells (e.g., white cells), tissue or fine needle biopsy samples (e.g., a tendon biopsy), and cells therefrom. Biological samples can also include sections of tissues, such as frozen sections or formalin fixed sections taken for histological purposes.

As used herein, the term “complementary” refers to two nucleotide sequences that comprise antiparallel nucleotide sequences capable of pairing with one another upon formation of hydrogen bonds between the complementary base residues in the antiparallel nucleotide sequences. As is known in the art, the nucleic acid sequences of two complementary strands are the reverse complement of each other when each is viewed in the 5′ to 3′ direction. Unless specifically indicated to the contrary, the term “complementary” as used herein refers to 100% complementarity throughout the length of at least one of the two antiparallel nucleotide sequences.

As used herein, the term “fragment” refers to a sequence that comprises a subset of another sequence. When used in the context of a nucleic acid or amino acid sequence, the terms “fragment” and “subsequence” are used interchangeably. A fragment of a nucleic acid sequence can be any number of nucleotides that is less than that found in another nucleic acid sequence, and thus includes, but is not limited to, the sequences of an exon or intron, a promoter, an enhancer, an origin of replication, a 5′ or 3′ untranslated region, a coding region, and/or a polypeptide binding domain. It is understood that a fragment or subsequence can also comprise less than the entirety of a nucleic acid sequence, for example, a portion of an exon or intron, promoter, enhancer, etc. Similarly, a fragment or subsequence of an amino acid sequence can be any number of residues that is less than that found in a naturally occurring polypeptide, and thus includes, but is not limited to, domains, features, repeats, etc. Also similarly, it is understood that a fragment or subsequence of an amino acid sequence need not comprise the entirety of the amino acid sequence of the domain, feature, repeat, etc.

As used herein, the term “gene” is used broadly to refer to any segment of DNA associated with a biological function. Thus, genes include, but are not limited to, coding sequences, the regulatory sequences required for their expression, intron sequences associates with the coding sequences, and combinations thereof. Genes can also include non-expressed DNA segments that, for example, form recognition sequences for a polypeptide. Genes can be obtained from a variety of sources, including cloning from a source of interest or synthesizing from known or predicted sequence information, and can include sequences designed to have desired parameters.

The terms “heterologous”, “recombinant”, and “exogenous”, when used herein to refer to a nucleic acid sequence (e.g., a DNA sequence) or a gene, refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or other recombinant techniques. The terms also include non-naturally occurring multiple copies of a naturally occurring DNA sequence. Thus, the terms refer to a DNA segment that is foreign to the cell, or homologous to the cell but in a position or form within the host cell in which the element is not ordinarily found. Similarly, when used in the context of a polypeptide or amino acid sequence, an exogenous polypeptide or amino acid sequence is a polypeptide or amino acid sequence that originates from a source foreign to the particular host cell or, if from the same source, is modified from its original form. Thus, exogenous DNA segments can be expressed to yield exogenous polypeptides.

An “endogenous” or “native” nucleic acid (or amino acid) sequence is a nucleic acid (or amino acid) sequence naturally associated with a host cell into which it is introduced. In this context, the terms “heterologous” and “endogenous” are antonymous.

The phrase “hybridizing specifically to” refers to the binding, duplexing, or hybridizing of a molecule only to a particular nucleotide sequence under stringent conditions when that sequence is present in a complex mixture (e.g., total cellular) of DNA and/or RNA. The phrase “bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

As used herein, the term “isolated”, when used in the context of an isolated nucleic acid or an isolated polypeptide, is a nucleic acid or polypeptide that, by the hand of man, exists apart from its native environment and is therefore not a product of nature. An isolated nucleic acid molecule or polypeptide can exist in a purified form or can exist in a non-native environment such as, for example, in a transformed host cell.

As used herein, the term “native” refers to a gene that is naturally present in the genome of an untransformed cell. Similarly, when used in the context of a polypeptide, a “native polypeptide” is a polypeptide that is encoded by a native gene of an untransformed cell's genome. Thus, the terms “native” and “endogenous” are synonymous.

As used herein, the term “naturally occurring” refers to an object that is found in nature as distinct from being artificially produced or manipulated by man. For example, a polypeptide or nucleotide sequence that is present in an organism (including a virus) in its natural state, which has not been intentionally modified or isolated by man in the laboratory, is naturally occurring. As such, a polypeptide or nucleotide sequence is considered “non-naturally occurring” if it is encoded by or present within a recombinant molecule, even if the amino acid or nucleic acid sequence is identical to an amino acid or nucleic acid sequence found in nature.

As used herein, the term “nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., 1991; Ohtsuka et al., 1985; Rossolini et al., 1994). The terms “nucleic acid” or “nucleic acid sequence” can also be used interchangeably with gene, cDNA, and mRNA encoded by a gene.

As used herein, the phrase “oligonucleotide” refers to a polymer of nucleotides of any length. In some embodiments, an oligonucleotide is a primer that is used in a polymerase chain reaction (PCR) and/or reverse transcription-polymerase chain reaction (RT-PCR), and the length of the oligonucleotide is typically between about 15 and 30 nucleotides. In some embodiments, the oligonucleotide is present on an array and is specific for a gene of interest. In whatever embodiment that an oligonucleotide is employed, one of ordinary skill in the art is capable of designing the oligonucleotide to be of sufficient length and sequence to be specific for the gene of interest (i.e., that would be expected to specifically bind only to a product of the gene of interest under a given hybridization condition).

As used herein, the phrase “percent identical”, in the context of two nucleic acid or polypeptide sequences, refers to two or more sequences or subsequences that have in some embodiments 60%, in some embodiments 70%, in some embodiments 75%, in some embodiments 80%, in some embodiments 85%, in some embodiments 90%, in some embodiments 92%, in some embodiments 94%, in some embodiments 95%, in some embodiments 96%, in some embodiments 97%, in some embodiments 98%, in some embodiments 99%, and in some embodiments 100% nucleotide or amino acid residue identity, respectively, when compared and aligned for maximum correspondence, as measured using one of the following sequence comparison algorithms or by visual inspection. The percent identity exists in some embodiments over a region of the sequences that is at least about 50 residues in length, in some embodiments over a region of at least about 100 residues, and in some embodiments, the percent identity exists over at least about 150 residues. In some embodiments, the percent identity exists over the entire length of the sequences.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm disclosed in Smith & Waterman, 1981; by the homology alignment algorithm disclosed in Needleman & Wunsch, 1970; by the search for similarity method disclosed in Pearson & Lipman, 1988; by computerized implementations of these algorithms (GAP, BESTFIT, FASTA, and TFASTA in the GCG® WISCONSIN PACKAGE®, available from Accelrys, Inc., San Diego, Calif., United States of America), or by visual inspection. See generally, Altschul et al., 1990; Ausubel et al., 2002; and Ausubel et al., 2003.

One example of an algorithm that is suitable for determining percent sequence identity and sequence similarity is the BLAST algorithm, which is described in Altschul et al., 1990. Software for performing BLAST analysis is publicly available through the website of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. See generally, Altschul et al., 1990. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences (see e.g., Karlin & Altschul, 1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

As used herein, the term “subject” refers to any organism for which analysis of gene expression would be desirable. Thus, the term “subject” is desirably a human subject, although it is to be understood that the principles of the presently disclosed subject matter indicate that the presently disclosed subject matter is effective with respect to invertebrate and to all vertebrate species, including mammals, which are intended to be included in the term “subject”. Moreover, a mammal is understood to include any mammalian species in which detection of differential gene expression is desirable, particularly agricultural and domestic mammalian species. The methods of the presently disclosed subject matter are particularly useful in the analysis of gene expression in warm-blooded vertebrates, e.g., mammals and birds.

More particularly, the presently disclosed subject matter can be used for the analysis of gene expression (e.g., connective tissue gene expression) in a mammal such as a human. Also provided is the analysis of gene expression in mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses (e.g., thoroughbreds and race horses). Also provided is the analysis of gene expression of birds, including those kinds of birds that are endangered, or kept in zoos, as well as fowl, and more particularly domesticated fowl, e.g., poultry, such as turkeys, chickens, ducks, geese, guinea fowl, quail, pheasant, and the like, as they are also of economic importance to humans. Thus, provided is the analysis of gene expression in livestock, including, but not limited to, domesticated swine (pigs and hogs), ruminants, poultry, and the like.

### II. ANALYSIS OF DIFFERENTIAL GENE EXPRESSION

Many biological functions are accomplished by altering the expression of various genes through transcriptional (e.g., through control of initiation, provision of RNA precursors, RNA processing, etc.) and/or translational control. For example, fundamental biological processes such as cell cycle, cell differentiation, and cell death, are often characterized by the variations in the expression levels of groups of genes.

Thus, differential gene expression can result in the differentiation of a pluripotent precursor cell into different cell types (e.g., the differentiation of tendon cells from pluripotent mesenchymal stem cells). As this differentiation takes place, unique combinations of genes are typically expressed in different terminally differentiated cell types, and the expression of these unique combinations of genes can be identified. As disclosed herein, genes that are differentially expressed in cells of connective tissue (e.g., tendon cells) as compared to cells of other related tissues (e.g., muscle cells) have been identified.

II.A. Identification of Connective Tissue-Specific Genes

The presently disclosed subject matter provides in some embodiments methods for identifying connective tissue-specific genes. As used herein, the phrase “connective tissue” refers to those tissues that are typically classified as soft connective tissues including, for example, tendons, ligaments, menisci, muscle, fascia, sheaths and the like. Included within the definition of “connective tissue” are terminally differentiated cells as well as precursor cells that have the potential to differentiate into connective tissue cells and tissues.

The presently disclosed subject matter provides in some embodiments methods for detecting tendon-specific gene expression in a sample. In some embodiments, the methods comprise detecting a level of expression in a sample of at least one gene listed in Tables 1-4, wherein the at least one gene is tendon-specific. In some embodiments, the methods comprise detecting a level of expression in a sample of at least 1, 2, 3, 4, 5, 6, 7, 8, 9, 10, 15, 20, 25, 50, or more of the genes listed in Tables 1-4, wherein the at least 1, 2, 3, 4, 5, 6, 7, 8, 9, 10, 15, 20, 25, 50, or more of the genes are tendon-specific. In some embodiments, the 1, 2, 3, 4, 5, 6, 7, 8, 9, 10, or more genes that are tendon-specific are listed in Table 1B.

As used herein, the phrase “tendon-specific” refers in some embodiments to a gene that is expressed in a tendon cell and for which expression in some or all other cell types is negligible. Thus, in some embodiments “tendon-specific” means that the gene in question is expressed only in a tendon cell or a precursor cell committed to tendon differentiation.

In some embodiments, however, “tendon-specific” refers to a gene that is upregulated and/or expressed at a higher level in tendon cells and their committed precursors relative to another cell type. An example of a tendon-specific gene within this meaning is mouse procollagen, type I, alpha 1 (Col1a1; GENBANK® Accession No. NM_{—}007742), which as disclosed in Table 1B is expressed in Achilles tendon at a level that is more than 16 fold higher than the gene is expressed in gastrocnemius muscle. Thus, in these embodiments “tendon-specific” is used in a relative sense and not in an absolute sense.

Exemplary tendon-specific genes include those genes listed in Tables 1-4. In some embodiments, a tendon-specific gene is selected from the group consisting of those genes listed in Tables 1B, 2B, and 3A.

II.B. Identification of Chances in Gene Expression under Different Genetic Influences

The presently disclosed subject matter also provides in some embodiments methods for analyzing differential gene expression in a cell or tissue type that result from genetic differences between subjects or in the same subject at different times (e.g., before an after the occurrence of a mutation). In some embodiments, the genetic differences result from a mutation in (e.g., a targeted disruption of) one or more genes the products of which are normally expressed in a connective tissue, such as tendon.

An example of a genetic influence relevant to tendon development is the activity of the metabotropic purinoceptors P2Y₁ and P2Y₂ (also referred to as P2RY₁ and P2RY₂). These receptors are coupled to G-protein coupled receptors that activate a phosphatidylinositol-calcium second messenger system in many cell types including tendon cells. Targeted disruption of P2Y₁, P2Y₂, or both P2Y₁ and P2Y₂ greatly influences gene expression in tendons, as shown in Examples 2 and 3 and Tables 2 and 3.

II.C. Identification of Changes in Gene Expression During Different Physiological Responses

The presently disclosed subject matter also provides in some embodiments methods for analyzing differential gene expression in a cell or tissue type in response to different environmental factors including, but not limited to disease, injury, exposure to bioactive molecules, and combinations thereof.

Connective tissues, such as tendons, are constantly being remodeled in subjects as a result of normal use, and particularly in the event of injury or disease. All of these conditions (e.g., normal use, injury, and/or disease) induce both catabolic and anabolic responses in connective tissues, often inducing anabolic responses followed by catabolic responses as the connective tissue recovers and/or heals. Thus, it is desirable to analyze how gene expression is affected by processes that result in catabolic and/or anabolic pathways in connective tissues, such as tendons.

In some embodiments, a technique to stimulate expression of marker gene expression that is indicative of a catabolic pathway is the application of hyperphysiologic levels of exercise as mechanical load. Mechanical load, when given in a hyper-physiologic dose results in pathology and can result in matrix degradation and loss of material properties. Hence, one assessment of potential negative effects of hyperphysiologic mechanical load is the tensile strength of the biologic material. One method to test such a property is to apply a tensile load to a biologic tissue at a controlled rate and force and apply load until the specimen fails. The characteristics of the stress train curve yield a quantitative assessment of the material's modulus or degree of stiffness.

Next, another strategy to stimulate expression marker gene expression that is indicative of a catabolic pathway that represents the environmental scenario induced during a pathologic response can be used. An example of a catabolic factor is interleukin 1β (IL-1β), which induces a group of matrix destructive genes called matrix metalloproteinases (MMPs). These MMPs degrade the material that lends tensile load bearing strength to most connective tissues, particularly to tendons.

To simulate catabolic responses in tendons, tendon cells can be isolated and exposed to IL-1β (for example, human tendon cells can be treated in vitro with recombinant human IL-1β). Differential gene expression analysis can then be employed to analyze how tendon cells respond to catabolic conditions, and the genes identified as being responsive to catabolic activity can be identified. This technique is disclosed in Example 4 and the genes so identified are presented in Table 4.

II.D. Other Applications

The genes and gene expression information provided herein, such as in Tables 1-4, can also be used as markers for the monitoring of disease and/or injury progression and/or the progress of a treatment, for instance, a recovery from an injury to a connective tissue, such as a tendon. For example, a tendon tissue sample or other sample from a patient can be assayed by any of the approaches disclosed herein, and the expression levels in the sample from a gene or genes from Tables 1-4 can be compared to the expression levels found in a reference tissue, e.g. normal tendon tissue and/or discarded or injured tissue. Comparison of the expression data, as well as available sequence or other information can be done by researcher or diagnostician or can be done with the aid of a computer and databases as described herein. Representative treatments include pharmacological treatments, physical therapy treatments, and combinations thereof.

The genes and gene expression information provided herein, such as in Tables 1-4, can also be used as markers for the diagnosis of connective tissue disease, for instance, a disease of a connective tissue such as a tendon. For example, a tendon tissue sample or other sample from a patient suspected of having a tendon disease can be assayed by any of the approaches disclosed herein, and the expression levels in the sample from a gene or genes from Tables 1-4 can be compared to the expression levels found in a reference tissue, e.g. normal tendon tissue (e.g., from another tendon in the same subject or a different subject).

Monitoring changes in gene expression can also provide certain advantages during drug screening development. Often drugs are screened and prescreened for the ability to interact with a major target without regard to other effects the drugs have on cells. Often such other effects cause toxicity in the whole animal, which prevent the development and use of the potential drug.

According to the presently disclosed subject matter, the genes disclosed herein, for example those disclosed in Tables 1-4, can also be used as markers to evaluate the effects of a candidate drug or agent on a connective tissue cell, such as but not limited to a tendon cell undergoing repair from injury or disease, such as for example, a tendon cell or tendon tissue sample. A candidate drug or agent can be screened for the ability to stimulate the transcription or expression of a given marker or markers (drug targets) or to down-regulate or counteract the transcription or expression of a marker or markers. According to the presently disclosed subject matter, one can also compare the specificity of a drug's effects by looking at the number of markers that the drugs have and comparing them. More specific drugs will have fewer transcriptional targets. Similar sets of markers identified for two drugs indicate a similarity of effects.

Assays to monitor the expression of a marker or markers disclosed herein, such as those defined in Tables 1-4, can utilize any available technique of monitoring for changes in the expression level of the biosequences disclosed herein. As used herein, an agent is said to modulate the expression of a biosequence if it is capable of up- or down-regulating expression of the biosequence in a cell.

In some embodiments, gene chips containing oligonucleotides that specifically bind to at least one, two, three, four, five, six, seven, eight, nine, ten, or more genes from a target cell type (e.g., those genes disclosed in Tables 1-4) can be used to directly monitor or detect changes in gene expression in the treated or exposed cell. In another format, cell lines that contain reporter gene fusions between the open reading frame and/or the 3′ or 5′ regulatory regions of a gene (e.g., those listed in Tables 1-4) and any assayable fusion partner can be prepared. Numerous assayable fusion partners are known and readily available including the firefly luciferase gene and the gene encoding chloramphenicol acetyltransferase (Alam et al., 1990). Cell lines containing the reporter gene fusions are then exposed to the agent to be tested under appropriate conditions and time. Differential expression of the reporter gene between samples exposed to the agent and control samples identifies agents that modulate the expression of the nucleic acid.

Additional assay formats can be used to monitor the ability of the agent to modulate the expression of a gene identified herein (e.g., in Tables 1-4). For instance, as described above, mRNA expression can be monitored directly by hybridization of probes to the biosequences disclosed herein. Cell lines are exposed to the agent to be tested under appropriate conditions and time and total RNA or mRNA is isolated by standard procedures such those disclosed in Sambrook and Russell, 2001.

In some embodiments, cells or cell lines are first identified which express the gene products disclosed herein physiologically. Cell and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Such cell lines can be, but are not required to be, derived from connective tissue, such as tendon. Further, such cells or cell lines can be transduced or transfected with an expression vehicle (e.g., a plasmid or viral vector) construct comprising an operable non-translated 5′-promoter containing end of the structural gene encoding the presently disclosed gene products fused to one or more antigenic fragments, which are peculiar to the presently disclosed gene products, wherein said fragments are under the transcriptional control of said promoter and are expressed as polypeptides whose molecular weight can be distinguished from the naturally occurring polypeptides or can further comprise an immunologically distinct tag. Such a process is known in the art (see Sambrook and Russell, 2001).

Cells or cell lines transduced or transfected as outlined above are then contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum, or conditioned media comprising PBS or BSS and serum incubated at 37° C. These conditions can be modulated as deemed necessary by one of skill in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation, or Western blot). The pool of proteins isolated from the “agent-contacted” sample can be compared with a control sample where only the excipient is contacted with the cells and an increase or decrease in the immunologically generated signal from the “agent-contacted” sample compared to the control can be used to distinguish the effectiveness of the agent.

In some embodiments, the presently disclosed subject matter provides methods for identifying agents that modulate the levels, concentration, or at least one activity of a protein(s) encoded by genes disclosed herein, such as in Tables 1-4. Such methods or assays can utilize any method of monitoring or detecting the desired activity.

In some embodiments, the relative amounts of a protein of the presently disclosed subject matter between a cell population that has been exposed to the agent to be tested compared to an unexposed control cell population can be assayed. In this format, probes such as specific antibodies are used to monitor the differential expression of the protein in the different cell populations. Cell lines or populations are exposed to the agent to be tested under appropriate conditions and time. Cellular lysates can be prepared from the exposed cell line or population and a control, unexposed cell line or population. The cellular lysates are then analyzed with the probe, such as a specific antibody.

Agents that are assayed in the above methods can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis, which takes into account the sequence of the target site and/or its conformation in connection with the agent's action. Agents can be rationally selected or rationally designed by utilizing the peptide sequences that make up these sites.

For example, a rationally selected peptide agent can be a peptide comprising an amino acid sequence identical to or a derivative of any functional consensus site.

The agents of the presently disclosed subject matter can include, but are not limited to peptides, small molecules, vitamin derivatives, and carbohydrates. Dominant negative proteins, DNA encoding these proteins, antibodies to these proteins, peptide fragments of these proteins, and/or mimics of these proteins can be introduced into cells to affect function. “Mimic” as used herein refers to the modification of a region or several regions of a peptide molecule to provide a structure chemically different from the parent peptide but topographically and functionally similar to the parent peptide (see Grant 1995). A skilled artisan can readily recognize that there is no limit as to the structural nature of the agents of the presently disclosed subject matter.

II.E. Methods of Gene Expression Analysis

II.E.1. Assay Formats

The genes identified as being differentially expressed in, for example, tendon cells versus muscle cells, or in tendon cells under different genetic or environmental conditions, can be used in a variety of nucleic acid detection assays to detect or quantitate the expression level of a gene or multiple genes in a given sample. For example, Northern blotting, nuclease protection, RT-PCR (e.g., quantitative RT-PCR; QRT-PCR), and/or differential display methods can be used for detecting gene expression levels. In some embodiments, methods and assays of the presently disclosed subject matter are employed with array or chip hybridization-based methods for detecting the expression of a plurality of genes.

Any hybridization assay format can be used, including solution-based and solid support-based assay formats. Representative solid supports containing oligonucleotide probes for differentially expressed genes of the presently disclosed subject matter can be filters, polyvinyl chloride dishes, silicon, glass based chips, etc. Such wafers and hybridization methods are widely available and include, for example, those disclosed in PCT International Patent Application Publication WO 95/11755). Any solid surface to which oligonucleotides can be bound, either directly or indirectly, either covalently or non-covalently, can be used. An exemplary solid support is a high-density array or DNA chip. These contain a particular oligonucleotide probe in a predetermined location on the array. Each predetermined location can contain more than one molecule of the probe, but in some embodiments each molecule within the predetermined location has an identical sequence. Such predetermined locations are termed features. There can be any number of features on a single solid support including, for example, about 2, 10, 100, 1000, 10,000, 100,000, or 400,000 of such features on a single solid support. The solid support, or the area within which the probes are attached, can be of any convenient size (for example, on the order of a square centimeter).

Oligonucleotide probe arrays for differential gene expression monitoring can be made and employed according to any techniques known in the art (see e.g., Lockhart et al, 1996; McGall et al, 1996). Such probe arrays can contain at least two or more oligonucleotides that are complementary to or hybridize to two or more of the genes described herein. Such arrays can also contain oligonucleotides that are complementary or hybridize to at least about 2, 3, 4, 5, 6, 7, 8, 9, 10, 15, 20, 25, 30, 50, 70, 100, or more of the nucleic acid sequences disclosed herein.

The genes that are assayed according to the presently disclosed subject matter are typically in the form of RNA (e.g., total RNA or mRNA) or reverse transcribed RNA. The genes can be cloned or not, and the genes can be amplified or not. In some embodiments, poly A⁺ RNA is employed as a source.

The sequences of the expression marker genes disclosed herein are in the public databases and/or are disclosed in the Sequence Listing. Tables 1-4 provide the GENBANK® Accession Numbers for the nucleic acid sequences identified. The sequences of the genes in the GENBANK® database are expressly incorporated by reference as are equivalent and related sequences present in GENBANK® or other public databases. Also expressly incorporated herein by reference are all annotations present in the GENBANK® database associated with the sequences disclosed herein.

It is understood, for example, that although Tables 1-3 disclose nucleic acid sequences from mouse and Table 4 discloses nucleic acid sequences from human, the techniques disclosed herein can be used to detect differential expression of the genes disclosed in Tables 1-4 for any species. For example, Table 1 discloses that Annexin A8 (Anxa8) is expressed to an about 10 fold higher level in mouse Achilles tendon than in mouse gastrocnemius muscle. The nucleic acid sequence of a mouse Anxa8 gene product is disclosed as corresponding to GENBANK® Accession No. NM_{—}013473. However, when the subject is a human subject, it is understood that the expression level of the human ANXA8 gene would be assayed, and reagents that are capable of detecting expression of a human ANXA8 gene product (e.g., an RNA transcribed from, or a polypeptide encoded by, human ANXA8) would be designed based upon the nucleic acid and/or amino acid sequences of human ANXA8. It is further understood that the nucleic acid and amino acid sequences of these gene products are also publicly available, for example in the GENBANK® database (Accession Nos. NM_{—}001630 and NP_{—}001621, respectively), as are the nucleic acid and amino acid sequences of the genes listed in Tables 1-4 from several species other than human and mouse. As such, sequences corresponding to the GENBANK® database entries explicitly recited herein, as well as all sequences corresponding to orthologous sequences in other species that are also present in the GENBANK® database, are incorporated herein by reference.

Probes based on the sequences of the genes described herein can be prepared by any commonly available method. Oligonucleotide probes for assaying the tissue or cell sample are in some embodiments of sufficient length to specifically hybridize only to appropriate, complementary genes or transcripts. Typically, the oligonucleotide probes are at least 10, 12, 14, 16, 18, 20, or 25 nucleotides in length. In some embodiments, longer probes of at least 30, 40, 50, or 60 nucleotides are employed.

As used herein, oligonucleotide sequences that are complementary to one or more of the genes described herein are oligonucleotides that are capable of hybridizing under stringent conditions to at least part of the nucleotide sequence of said genes. Such hybridizable oligonucleotides will typically exhibit in some embodiments at least about 75% sequence identity, in some embodiments about 80% sequence identity, in some embodiments about 85% sequence identity, in some embodiments about 90% sequence identity, in some embodiments about 95% sequence identity, and in some embodiments greater than 95% sequence identity (e.g., 96%, 97%, 98%, 99%, or 100% sequence identity) at the nucleotide level to the nucleic acid sequences disclosed herein.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals can also be produced by intrinsic fluorescence of the array components themselves. A single background signal can be calculated for the entire array, or a different background signal can be calculated for each target nucleic acid. In some embodiments, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample such as bacterial genes where the sample is mammalian nucleic acids). Background can also be calculated as the average signal intensity produced by regions of the array that lack any probes at all.

Assays and methods of the presently disclosed subject matter can utilize available formats to simultaneously screen in some embodiments at least about 10, in some embodiments at least about 50, in some embodiments at least about 100, in some embodiments at least about 1000, in some embodiments at least about 10,000, and in some embodiments at least about 40,000 or more different nucleic acid hybridizations.

The terms “mismatch control” and “mismatch probe” refer to a probe comprising a sequence that is deliberately selected not to be perfectly complementary to a particular target sequence. For each mismatch (MM) control in a high-density array there typically exists a corresponding perfect match (PM) probe that is perfectly complementary to the same particular target sequence. The mismatch can comprise one or more bases.

While the mismatch(s) can be located anywhere in the mismatch probe, terminal mismatches are less desirable as a terminal mismatch is less likely to prevent hybridization of the target sequence. In some embodiments, the mismatch is located at or near the center of the probe such that the mismatch is most likely to destabilize the duplex with the target sequence under the test hybridization conditions.

The phrase “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe, or the like. A perfect match control or perfect match probe is, however, distinguished from a “mismatch control” or “mismatch probe”.

As used herein, a “probe” is defined as a nucleic acid that is capable of binding to a target nucleic acid of complementary sequence through one or more types of chemical bonds, usually through complementary base pairing, usually through hydrogen bond formation. As used herein, a probe can include natural (i.e., A, G, U, C, or T) or modified bases (7-deazaguanosine, inosine, etc.). In addition, the bases in probes can be joined by a linkage other than a phosphodiester bond, so long as it does not interfere with hybridization. Thus, probes can be peptide nucleic acids in which the constituent bases are joined by peptide bonds rather than phosphodiester linkages.

II.E.2. Probe Design

Upon review of the present disclosure, one of skill in the art will appreciate that an enormous number of array designs are suitable for the practice of the presently disclosed subject matter. The high-density array typically includes a number of probes that specifically hybridize to the sequences of interest. See PCT International Patent Application Publication WO 99/32660, incorporated herein be reference in its entirety, for methods of producing probes for a given gene or genes. In addition, in some embodiments, the array includes one or more control probes.

High-density array chips of the presently disclosed subject matter include in some embodiments “test probes”. Test probes can be oligonucleotides that in some embodiments range from about 5 to about 500 or about 5 to about 50 nucleotides, in some embodiments from about 10 to about 40 nucleotides, and in some embodiments from about 15 to about 40 nucleotides in length. In some embodiments, the probes are about 20 to 25 nucleotides in length. In some embodiments, test probes are double or single strand DNA sequences. DNA sequences are isolated or cloned from natural sources and/or amplified from natural sources using natural nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high-density array can contain a number of control probes. The control probes fall into three categories referred to herein as (1) normalization controls; (2) expression level controls; and (3) mismatch controls.

Normalization controls are oligonucleotide or other nucleic acid probes that are complementary to labeled reference oligonucleotides or other nucleic acid sequences that are added to the nucleic acid sample. The signals obtained from the normalization controls after hybridization provide a control for variations in hybridization conditions, label intensity, “reading” efficiency and other factors that can cause the signal of a perfect hybridization to vary between arrays. In some embodiments, signals (e.g., fluorescence intensity) read from all other probes in the array are divided by the signal (e.g., fluorescence intensity) from the control probes, thereby normalizing the measurements.

Virtually any probe can serve as a normalization control. However, it is recognized that hybridization efficiency varies with base composition and probe length. Exemplary normalization probes can be selected to reflect the average length of the other probes present in the array; however, they can be selected to cover a range of lengths. The normalization control(s) can also be selected to reflect the (average) base composition of the other probes in the array; however, in some embodiments, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typical expression level control probes have sequences complementary to subsequences of constitutively expressed “housekeeping genes” including, but not limited to, the β-actin gene, the transferrin receptor gene, the GAPDH gene, and the like.

Mismatch controls can also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). In some embodiments, mismatch probes contain one or more central mismatches. Thus, for example, where a probe is a 20-mer, a corresponding mismatch probe will have the identical sequence except for a single base mismatch (e.g., substituting a G, a C, or a T for an A) at any of positions 6 through 14 (the central mismatch).

Mismatch probes thus provide a control for non-specific binding or cross hybridization to a nucleic acid in the sample other than the target to which the probe is directed. Mismatch probes also indicate whether a hybridization is specific or not. For example, if the target is present the perfect match probes should be consistently brighter than the mismatch probes. In addition, if all central mismatches are present, the mismatch probes can be used to detect a mutation. The difference in intensity between the perfect match and the mismatch probe (IBM)-I(MM)) provides a good measure of the concentration of the hybridized material.

II.E.3. Nucleic Acid Samples

A biological sample that can be analyzed in accordance with the presently disclosed subject matter comprises in some embodiments a nucleic acid. The terms “nucleic acid”, “nucleic acids”, and “nucleic acid molecules” each refer in some embodiments to deoxyribonucleotides, ribonucleotides, and polymers and folded structures thereof in either single- or double-stranded form. Nucleic acids can be derived from any source, including any organism. Deoxyribonucleic acids can comprise genomic DNA, cDNA derived from ribonucleic acid, DNA from an organelle (e.g., mitochondrial DNA or chloroplast DNA), or combinations thereof. Ribonucleic acids can comprise genomic RNA (e.g., viral genomic RNA), messenger RNA (mRNA), ribosomal RNA (rRNA), transfer RNA (tRNA), or combinations thereof.

II.E.3.i. Isolation of Nucleic Acid Samples

Nucleic acid samples used in the methods and assays of the presently disclosed subject matter can be prepared by any available method or process. Methods of isolating total mRNA are also known to those of skill in the art. For example, methods of isolation and purification of nucleic acids are described in detail in Chapter 3 of Tijssen 1993. Such samples include RNA samples, but also include cDNA synthesized from an mRNA sample isolated from a cell or tissue of interest. Such samples also include DNA amplified from the cDNA, an RNA transcribed from the amplified DNA, and combinations thereof. One of skill in the art would appreciate that it can be desirable to inhibit or destroy RNase present in homogenates before homogenates are used as a source of RNA.

The presently disclosed subject matter encompasses use of a sufficiently large biological sample to enable a comprehensive survey of low abundance nucleic acids in the sample. Thus, the sample can optionally be concentrated prior to isolation of nucleic acids. Several protocols for concentration have been developed that alternatively use slide supports (Kohsaka & Carson, 1994; Millar et al., 1995), filtration columns (Bej et al., 1991), or immunomagnetic beads (Albert et al., 1992; Chiodi et al., 1992). Such approaches can significantly increase the sensitivity of subsequent detection methods.

As one example, SEPHADEX® matrix (Sigma of St. Louis, Mo., United States of America) is a matrix of diatomaceous earth and glass suspended in a solution of chaotropic agents and has been used to bind nucleic acid material (Boom et al., 1990; Buffone et al., 1991). After the nucleic acid is bound to the solid support material, impurities and inhibitors are removed by washing and centrifugation, and the nucleic acid is then eluted into a standard buffer. Target capture also allows the target sample to be concentrated into a minimal volume, facilitating the automation and reproducibility of subsequent analyses (Lanciotti et al., 1992).

Methods for nucleic acid isolation can comprise simultaneous isolation of total nucleic acid, or separate and/or sequential isolation of individual nucleic acid types (e.g., genomic DNA, cDNA, organelle DNA, genomic RNA, mRNA, poly A⁺ RNA, rRNA, tRNA) followed by optional combination of multiple nucleic acid types into a single sample.

When RNA (e.g., mRNA) is selected for analysis, the disclosed methods allow for an assessment of gene expression in the tissue or cell type from which the RNA was isolated. RNA isolation methods are known to one of skill in the art. See Albert et al., 1992; Busch et al., 1992; Hamel et al., 1995; Herrewegh et al., 1995; Izraeli et al., 1991; McCaustland et al., 1991; Natarajan et al., 1994; Rupp et al., 1988; Tanaka et al., 1994; and Vankerckhoven et al., 1994. A representative procedure for RNA isolation from a clinical sample is set forth in Example 1.

Simple and semi-automated extraction methods can also be used for nucleic acid isolation, including for example, the SPLIT SECONDT™ system (Boehringer Mannheim of Indianapolis, Ind., United States of America), the TRIZOL™ Reagent system (Life Technologies of Gaithersburg, Md., United States of America), and the FASTPREP™ system (Bio 101 of La Jolla, Calif., United States of America). See also Smith 1998; and Paladichuk 1999.

In some embodiments, nucleic acids that are used for subsequent amplification and labeling are analytically pure as determined by spectrophotometric measurements or by visual inspection following electrophoretic resolution. In some embodiments, the nucleic acid sample is free of contaminants such as polysaccharides, proteins, and inhibitors of enzyme reactions. When a biological sample comprises an RNA molecule that is intended for use in producing a probe, it is preferably free of DNase and RNase. Contaminants and inhibitors can be removed or substantially reduced using resins for DNA extraction (e.g., CHELEX™ 100 from BioRad Laboratories of Hercules, Calif., United States of America) or by standard phenol extraction and ethanol precipitation.

II.E.3.ii. Amplification of Nucleic Acid Samples

In some embodiments, a nucleic acid isolated from a biological sample is amplified prior to being used in the methods disclosed herein. In some embodiments, the nucleic acid is an RNA molecule, which is converted to a complementary DNA (cDNA) prior to amplification. Techniques for the isolation of RNA molecules and the production of cDNA molecules from the RNA molecules are known (see generally, Silhavy et al., 1984; Sambrook & Russell, 2001; Ausubel et al., 2002; and Ausubel et al., 2003). In some embodiments, the amplification of RNA molecules isolated from a biological sample is a quantitative amplification (e.g., by quantitative RT-PCR).

The terms “template nucleic acid” and “target nucleic acid” as used herein each refer to nucleic acids isolated from a biological sample as described herein above. The terms “template nucleic acid pool”, “template pool”, “target nucleic acid pool”, and “target pool” each refer to an amplified sample of “template nucleic acid”. Thus, a target pool comprises amplicons generated by performing an amplification reaction using the template nucleic acid. In some embodiments, a target pool is amplified using a random amplification procedure as described herein.

The term “target-specific primer” refers to a primer that hybridizes selectively and predictably to a target sequence, for example a tendon-specific sequence, in a target nucleic acid sample. A target-specific primer can be selected or synthesized to be complementary to known nucleotide sequences of target nucleic acids.

The term “random primer” refers to a primer having an arbitrary sequence. The nucleotide sequence of a random primer can be known, although such sequence is considered arbitrary in that it is not specifically designed for complementarity to a nucleotide sequence of the presently disclosed subject matter. The term “random primer” encompasses selection of an arbitrary sequence having increased probability to be efficiently utilized in an amplification reaction. For example, the Random Oligonucleotide Construction Kit (ROCK) is a macro-based program that facilitates the generation and analysis of random oligonucleotide primers (Strain & Chmielewski, 2001). Representative primers include but are not limited to random hexamers and rapid amplification of polymorphic DNA (RAPD)-type primers as described by Williams et al., 1990.

A random primer can also be degenerate or partially degenerate as described by Telenius et al., 1992. Briefly, degeneracy can be introduced by selection of alternate oligonucleotide sequences that can encode a same amino acid sequence.

In some embodiments, random primers can be prepared by shearing or digesting a portion of the template nucleic acid sample. Random primers so-constructed comprise a sample-specific set of random primers.

The term “heterologous primer” refers to a primer complementary to a sequence that has been introduced into the template nucleic acid pool. For example, a primer that is complementary to a linker or adaptor, as described below, is a heterologous primer. Representative heterologous primers can optionally include a poly(dT) primer, a poly(T) primer, or as appropriate, a poly(dA) or poly(A) primer.

The term “primer” as used herein refers to a contiguous sequence comprising in some embodiments about 6 or more nucleotides, in some embodiments about 10-20 nucleotides (e.g., 15-mer), and in some embodiments about 20-30 nucleotides (e.g., a 22-mer). Primers used to perform the methods of the presently disclosed subject matter encompass oligonucleotides of sufficient length and appropriate sequence so as to provide initiation of polymerization on a nucleic acid molecule.

U.S. Pat. No. 6,066,457 to Hampson et al. describes a method for substantially uniform amplification of a collection of single stranded nucleic acid molecules such as RNA. Briefly, the nucleic acid starting material is anchored and processed to produce a mixture of directional shorter random size DNA molecules suitable for amplification of the sample.

In accordance with the methods of the presently disclosed subject matter, any PCR technique or related technique can be employed to perform the step of amplifying the nucleic acid sample. In addition, such methods can be optimized for amplification of a particular subset of nucleic acid (e.g., genomic DNA versus RNA), and representative optimization criteria and related guidance can be found in the art. See Cha & Thilly, 1993; Linz et al., 1990; Robertson & Walsh-Weller, 1998; Roux 1995; Williams 1989; and McPherson et al., 1995.

II.E.3.iii. Labeling of Nucleic Acid Samples

Optionally, a nucleic acid sample (e.g., a quantitatively amplified RNA sample) further comprises a detectable label. In some embodiments of the presently disclosed subject matter, the amplified nucleic acids can be labeled prior to hybridization to an array. Alternatively, randomly amplified nucleic acids are hybridized with a set of probes, without prior labeling of the amplified nucleic acids. For example, an unlabeled nucleic acid in the biological sample can be detected by hybridization to a labeled probe. In some embodiments, both the randomly amplified nucleic acids and the one or more pathogen-specific probes include a label, wherein the proximity of the labels following hybridization enables detection. An exemplary procedure using nucleic acids labeled with chromophores and fluorophores to generate detectable photonic structures is described in U.S. Pat. No. 6,162,603 to Heller.

In accordance with the methods of the presently disclosed subject matter, the amplified nucleic acids or pathogen-specific probes/probe sets can be labeled using any detectable label. It will be understood to one of skill in the art that any suitable method for labeling can be used, and no particular detectable label or technique for labeling should be construed as a limitation of the disclosed methods.

Direct labeling techniques include incorporation of radioisotopic or fluorescent nucleotide analogues into nucleic acids by enzymatic synthesis in the presence of labeled nucleotides or labeled PCR primers. A radio-isotopic label can be detected using autoradiography or phosphorimaging. A fluorescent label can be detected directly using emission and absorbance spectra that are appropriate for the particular label used. Any detectable fluorescent dye can be used, including but not limited to FITC (fluorescein isothiocyanate), FLUOR X™, ALEXA FLUOR® 488, OREGON GREEN® 488, 6-JOE (6-carboxy-4′,5′-dichloro-2′,7′-dimethoxyfluorescein, succinimidyl ester), ALEXA FLUOR® 532, Cy3, ALEXA FLUOR® 546, TMR (tetramethylrhodamine), ALEXA FLUOR® 568, ROX (X-rhodamine), ALEXA FLUOR® 594, TEXAS RED®, BODIPY® 630/650, and Cy5 (available from Amersham Pharmacia Biotech of Piscataway, N.J., United States of America or from Molecular Probes Inc. of Eugene, Oreg., United States of America). Fluorescent tags also include sulfonated cyanine dyes (available from Li-Cor, Inc. of Lincoln, Nebr., United States of America) that can be detected using infrared imaging. Methods for direct labeling of a heterogeneous nucleic acid sample are known in the art and representative protocols can be found in, for example, DeRisi et al., 1996; Sapolsky & Lipshutz, 1996; Schena et al., 1995; Schena et al., 1996; Shalon et al., 1996; Shoemaker et al., 1996; and Wang et al., 1998.

In some embodiments, nucleic acid molecules isolated from different cell types and/or cell types from different genetic and/or environmental backgrounds are labeled with different detectable markers, allowing the nucleic acids to analyzed simultaneously on an array. For example, as disclosed in EXAMPLE 1, a first RNA sample (e.g., mouse Achilles tendon (AT) RNAs) can be reverse transcribed into cDNAs labeled with cyanine 3 (a green dye fluorophore; Cy3) while a second RNA sample to which the first RNA sample is to be compared (e.g., gastrocnemius muscle (GM) RNAs) can be labeled with cyanine 5 (a red dye fluorophore; Cy5).

The quality of probe or nucleic acid sample labeling can be approximated by determining the specific activity of label incorporation. For example, in the case of a fluorescent label, the specific activity of incorporation can be determined by the absorbance at 260 nm and 550 nm (for Cy3) or 650 nm (for Cy5) using published extinction coefficients (Randolph & Waggoner, 1995). Very high label incorporation (specific activities of >1 fluorescent molecule/20 nucleotides) can result in a decreased hybridization signal compared with probe with lower label incorporation. Very low specific activity (<1 fluorescent molecule/100 nucleotides) can give unacceptably low hybridization signals. See Worley et al., 2000. Thus, it will be understood to one of skill in the art that labeling methods can be optimized for performance in microarray hybridization assay, and that optimal labeling can be unique to each label type.

II.E.4. Forming High-Density Arrays

In some embodiments of the presently disclosed subject matter, probes or probe sets are immobilized on a solid support such that a position on the support identifies a particular probe or probe set. In the case of a probe set, constituent probes of the probe set can be combined prior to placement on the solid support or by serial placement of constituent probes at a same position on the solid support.

A microarray can be assembled using any suitable method known to one of skill in the art, and any one microarray configuration or method of construction is not considered to be a limitation of the presently disclosed subject matter. Representative microarray formats that can be used in accordance with the methods of the presently disclosed subject matter are described herein below and include, but are not limited to light-directed chemical coupling, and mechanically directed coupling (see U.S. Pat. Nos. 5,143,854 to Pirrung et al.; 5,800,992 to Fodor et al.; and 5,837,832 to Chee et al.).

II.E.4.i. Array Substrate and Configuration

The substrate for printing the array should be substantially rigid and amenable to DNA immobilization and detection methods (e.g., in the case of fluorescent detection, the substrate must have low background fluorescence in the region of the fluorescent dye excitation wavelengths). The substrate can be nonporous or porous as determined most suitable for a particular application. Representative substrates include but are not limited to a glass microscope slide, a glass coverslip, silicon, plastic, a polymer matrix, an agar gel, a polyacrylamide gel, and a membrane, such as a nylon, nitrocellulose or ANAPORE™ (Whatman of Maidstone, United Kingdom) membrane.

Porous substrates (membranes and polymer matrices) are preferred in that they permit immobilization of relatively large amount of probe molecules and provide a three-dimensional hydrophilic environment for biomolecular interactions to occur (Dubiley et al., 1997; Yershov et al., 1996). A BIOCHIP ARRAYER™ dispenser (Packard Instrument Company of Meriden, Conn., United States of America) can effectively dispense probes onto membranes such that the spot size is consistent among spots whether one, two, or four droplets were dispensed per spot (Englert 2000).

A microarray substrate for use in accordance with the methods of the presently disclosed subject matter can have either a two-dimensional (planar) or a three-dimensional (non-planar) configuration. An exemplary three-dimensional microarray is the FLOW-THRU™ chip (Gene Logic, Inc. of Gaithersburg, Md., United States of America), which has implemented a gel pad to create a third dimension. Such a three-dimensional microarray can be constructed of any suitable substrate, including glass capillary, silicon, metal oxide filters, or porous polymers. See Yang et al., 1998.

Briefly, a FLOW-THRU™ chip (Gene Logic, Inc.) comprises a uniformly porous substrate having pores or microchannels connecting upper and lower faces of the chip. Probes are immobilized on the walls of the microchannels and a hybridization solution comprising sample nucleic acids can flow through the microchannels. This configuration increases the capacity for probe and target binding by providing additional surface relative to two-dimensional arrays. See U.S. Pat. No. 5,843,767 to Beattie.

II.E.4.ii. Surface Chemistry

The particular surface chemistry employed is inherent in the microarray substrate and substrate preparation. Probe immobilization of nucleic acids probes post-synthesis can be accomplished by various approaches, including adsorption, entrapment, and covalent attachment. Typically, the binding technique is designed to not disrupt the activity of the probe.

For substantially permanent immobilization, covalent attachment is generally performed. Since few organic functional groups react with an activated silica surface, an intermediate layer is advisable for substantially permanent probe immobilization. Functionalized organosilanes can be used as such an intermediate layer on glass and silicon substrates (Liu & Hlady, 1996; Shriver-Lake 1998). A hetero-bifunctional cross-linker requires that the probe have a different chemistry than the surface, and is preferred to avoid linking reactive groups of the same type. A representative hetero-bifunctional cross-linker comprises gamma-maleimidobutyryloxy-succimide (GMBS) that can bind maleimide to a primary amine of a probe. Procedures for using such linkers are known to one of skill in the art and are summarized by Hermanson 1990. A representative protocol for covalent attachment of DNA to silicon wafers is described by O'Donnell et al., 1997.

When using a glass substrate, the glass should be substantially free of debris and other deposits and have a substantially uniform coating. Pretreatment of slides to remove organic compounds that can be deposited during their manufacture can be accomplished, for example, by washing in hot nitric acid. Cleaned slides can then be coated with 3-aminopropyltrimethoxysilane using vapor-phase techniques. After silane deposition, slides are washed with deionized water to remove any silane that is not attached to the glass and to catalyze unreacted methoxy groups to cross-link to neighboring silane moieties on the slide. The uniformity of the coating can be assessed by known methods, for example electron spectroscopy for chemical analysis (ESCA) or ellipsometry (Ratner & Castner, 1997; Schena et al., 1995). See also Worley et al., 2000.

For attachment of probes greater than about 300 base pairs, noncovalent binding is suitable. A representative technique for noncovalent linkage involves use of sodium isothiocyanate (NaSCN) in the spotting solution. When using this method, amino-silanized slides are typically employed because this coating improves nucleic acid binding when compared to bare glass. This method works well for spotting applications that use about 100 ng/μl (Worley et al., 2000).

In the case of nitrocellulose or nylon membranes, the chemistry of nucleic acid binding chemistry to these membranes has been well characterized (Southern 1975; Sambrook and Russell, 2001).

II.E.4.iii. Arraying Techniques

A microarray for the detection of pathogens in a biological sample can be constructed using any one of several methods available in the art, including but not limited to photolithographic and microfluidic methods, further described herein below. In some embodiments, the method of construction is flexible, such that a microarray can be tailored for a particular purpose.

As is standard in the art, a technique for making a microarray should create consistent and reproducible spots. Each spot is preferably uniform, and appropriately spaced away from other spots within the configuration. A solid support for use in the presently disclosed subject matter comprises in some embodiments about 10 or more spots, in some embodiments about 100 or more spots, in some embodiments about 1,000 or more spots, and in some embodiments about 10,000 or more spots. In some embodiments, the volume deposited per spot is about 10 picoliters to about 10 nanoliters, and in some embodiments about 50 picoliters to about 500 picoliters. The diameter of a spot is in some embodiments about 50 μm to about 1000 μm, and in some embodiments about 100 μm to about 250 μm.

Light-directed synthesis. This technique was developed by Fodor et al. (Fodor et al., 1991; Fodor et al., 1993), and commercialized by Affymetrix of Santa Clara, Calif., United States of America. Briefly, the technique uses precision photolithographic masks to define the positions at which single, specific nucleotides are added to growing single-stranded nucleic acid chains. Through a stepwise series of defined nucleotide additions and light-directed chemical linking steps, high-density arrays of defined oligonucleotides are synthesized on a solid substrate. A variation of the method, called Digital Optical Chemistry, employs mirrors to direct light synthesis in place of photolithographic masks (PCT International Patent Application Publication No. WO 99/63385). This approach is generally limited to probes of about 25 nucleotides in length or less. See also Warrington et al., 2000.

Contact Printing. Several procedures and tools have been developed for printing microarrays using rigid pin tools. In surface contact printing, the pin tools are dipped into a sample solution, resulting in the transfer of a small volume of fluid onto the tip of the pins. Touching the pins or pin samples onto a microarray surface leaves a spot, the diameter of which is determined by the surface energies of the pin, fluid, and microarray surface. Typically, the transferred fluid comprises a volume in the nanoliter or picoliter range.

One common contact printing technique uses a solid pin replicator. A replicator pin is a tool for picking up a sample from one stationary location and transporting it to a defined location on a solid support. A typical configuration for a replicating head is an array of solid pins, generally in an 8×12 format, spaced at 9-mm centers that are compatible with 96- and 384-well plates. The pins are dipped into the wells, lifted, moved to a position over the microarray substrate, lowered to touch the solid support, whereby the sample is transferred. The process is repeated to complete transfer of all the samples. See Maier et al., 1994. A recent modification of solid pins involves the use of solid pin tips having concave bottoms, which print more efficiently than flat pins in some circumstances. See Rose 2000.

Solid pins for microarray printing can be purchased, for example, from TeleChem International, Inc. of Sunnyvale, Calif. in a wide range of tip dimensions. The CHIPMAKER™ and STEALTH™ pins from TeleChem contain a stainless steel shaft with a fine point. A narrow gap is machined into the point to serve as a reservoir for sample loading and spotting. The pins have a loading volume of 0.2 μl to 0.6 μl to create spot sizes ranging from 75 μm to 360 μm in diameter.

To permit the printing of multiple arrays with a single sample loading, quill-based array tools, including printing capillaries, tweezers, and split pins have been developed. These printing tools hold larger sample volumes than solid pins and therefore allow the printing of multiple arrays following a single sample loading. Quill-based arrayers withdraw a small volume of fluid into a depositing device from a microwell plate by capillary action. See Schena et al., 1995. The diameter of the capillary typically ranges from about 10 μm to about 100 μm. A robot then moves the head with quills to the desired location for dispensing. The quill carries the sample to all spotting locations, where a fraction of the sample is deposited. The forces acting on the fluid held in the quill must be overcome for the fluid to be released. Accelerating and then decelerating by impacting the quill on a microarray substrate accomplishes fluid release. When the tip of the quill hits the solid support, the meniscus is extended beyond the tip and transferred onto the substrate. Carrying a large volume of sample fluid minimizes spotting variability between arrays. Because tapping on the surface is required for fluid transfer, a relatively rigid support, for example a glass slide, is appropriate for this method of sample delivery.

A variation of the pin printing process is the PIN-AND-RING™ technique developed by Genetic MicroSystems Inc. of Woburn, Mass., United States of America. This technique involves dipping a small ring into the sample well and removing it to capture liquid in the ring. A solid pin is then pushed through the sample in the ring, and the sample trapped on the flat end of the pin is deposited onto the surface. See Mace et al., 2000. The PIN-AND-RING™ technique is suitable for spotting onto rigid supports or soft substrates such as agar, gels, nitrocellulose, and nylon. A representative instrument that employs the PIN-AND-RING™ technique is the 417™ Arrayer available from Affymetrix of Santa Clara, Calif., United States of America.

Additional procedural considerations relevant to contact printing methods, including array layout options, print area, print head configurations, sample loading, preprinting, microarray surface properties, sample solution properties, pin velocity, pin washing, printing time, reproducibility, and printing throughput are known in the art, and are summarized by Rose 2000.

Noncontact Ink-Jet Printing. A representative method for noncontact ink-jet printing uses a piezoelectric crystal closely apposed to the fluid reservoir. One configuration places the piezoelectric crystal in contact with a glass capillary that holds the sample fluid. The sample is drawn up into the reservoir and the crystal is biased with a voltage, which causes the crystal to deform, squeeze the capillary, and eject a small amount of fluid from the tip. Piezoelectric pumps offer the capability of controllable, fast jetting rates and consistent volume deposition. Most piezoelectric pumps are unidirectional pumps that need to be directly connected, for example by flexible capillary tubing, to a source of sample supply or wash solution. The capillary and jet orifices should be of sufficient inner diameter so that molecules are not sheared. The void volume of fluid contained in the capillary typically ranges from about 100 μl to about 500 μl and generally is not recoverable. See U.S. Pat. No. 5,965,352 to Stoughton & Friend.

Devices that provide thermal pressure, sonic pressure, or oscillatory pressure on a liquid stream or surface can also be used for ink-jet printing. See Theriault et al., 1999.

Syringe-Solenoid Printing. Syringe-solenoid technology combines a syringe pump with a microsolenoid valve to provide quantitative dispensing of nanoliter sample volumes. A high-resolution syringe pump is connected to both a high-speed microsolenoid valve and a reservoir through a switching valve. For printing microarrays, the system is filled with a system fluid, typically water, and the syringe is connected to the microsolenoid valve. Withdrawing the syringe causes the sample to move upward into the tip. The syringe then pressurizes the system such that opening the microsolenoid valve causes droplets to be ejected onto the surface. With this configuration, a minimum dispense volume is on the order of 4 nl to 8 nl. The positive displacement nature of the dispensing mechanism creates a substantially reliable system. See U.S. Pat. Nos. 5,743,960 and 5,916,524, both to Tisone.

Electronic Addressing. This method involves placing charged molecules at specific positions on a blank microarray substrate, for example a NANOCHIP™ substrate (Nanogen Inc. of San Diego, Calif., United States of America). A nucleic acid probe is introduced to the microchip, and the negatively-charged probe moves to the selected charged position, where it is concentrated and bound. Serial application of different probes can be performed to assemble an array of probes at distinct positions. See U.S. Pat. No. 6,225,059 to Ackley et al. and PCT International Patent Application Publication No. WO 01/23082.

Nanoelectrode Synthesis. An alternative array that can also be used in accordance with the methods of the presently disclosed subject matter provides ultra small structures (nanostructures) of a single or a few atomic layers synthesized on a semiconductor surface such as silicon. The nanostructures can be designed to correspond precisely to the three-dimensional shape and electrochemical properties of molecules, and thus can be used to recognize nucleic acids of a particular nucleotide sequence. See U.S. Pat. No. 6,123,819 to Peeters.

In brief, the light-directed combinatorial synthesis of oligonucleotide arrays on a glass surface proceeds using automated phosphoramidite chemistry and chip masking techniques. In some embodiments, a glass surface is derivatized with a silane reagent containing a functional group, e.g., a hydroxyl or amine group blocked by a photolabile protecting group. Photolysis through a photolithogaphic mask is used selectively to expose functional groups that are then ready to react with incoming 5′ photoprotected nucleoside phosphoramidites. The phosphoramidites react only with those sites that are illuminated (and thus exposed by removal of the photolabile blocking group). Thus, the phosphoramidites only add to those areas selectively exposed from the preceding step. These steps are repeated until the desired array of sequences has been synthesized on the solid surface. Combinatorial synthesis of different oligonucleotide analogues at different locations on the array is determined by the pattern of illumination during synthesis and the order of addition of coupling reagents.

In addition to the foregoing, other methods that can be used to generate an array of oligonucleotides on a single substrate are described in PCT International Patent Application Publication WO 93/09668. High-density nucleic acid arrays can also be fabricated by depositing pre-made and/or natural nucleic acids in predetermined positions. Synthesized or natural nucleic acids are deposited on specific locations of a substrate by light directed targeting and oligonucleotide directed targeting. A dispenser that moves from region to region to deposit nucleic acids in specific spots can also be employed.

II.E.5. Hybridization

II.E.5.i. General Considerations

The terms “specifically hybridizes” and “selectively hybridizes” each refer to binding, duplexing, or hybridizing of a molecule only to a particular nucleotide sequence under stringent conditions when that sequence is present in a complex nucleic acid mixture (e.g., total cellular DNA or RNA).

The phrase “substantially hybridizes” refers to complementary hybridization between a probe nucleic acid molecule and a substantially identical target nucleic acid molecule as defined herein. Substantial hybridization is generally permitted by reducing the stringency of the hybridization conditions using art-recognized techniques.

“Stringent hybridization conditions” and “stringent hybridization wash conditions” in the context of nucleic acid hybridization experiments are both sequence- and environment-dependent. Longer sequences hybridize specifically at higher temperatures. Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. Typically, under “stringent conditions” a probe hybridizes specifically to its target sequence, but to no other sequences.

An extensive guide to the hybridization of nucleic acids is found in Tijssen 1993. In general, a signal to noise ratio of 2-fold (or higher) than that observed for a negative control probe in a same hybridization assay indicates detection of specific or substantial hybridization.

II.E.5.ii. Hybridization on a Solid Support

In some embodiments of the presently disclosed subject matter, an amplified and/or labeled nucleic acid sample is hybridized to specific probes or probe sets that are immobilized on a continuous solid support comprising a plurality of identifying positions. Representative formats of such solid supports are described herein.

The following are examples of hybridization and wash conditions that can be used to clone homologous nucleotide sequences that are substantially identical to reference nucleotide sequences of the presently disclosed subject matter: a probe nucleotide sequence hybridizes in one example to a target nucleotide sequence in 7% sodium dodecyl sulfate (SDS), 0.5M NaPO₄, 1 mm ethylene diamine tetraacetic acid (EDTA), 1% BSA at 50° C. followed by washing in 2×SSC, 0.1% SDS at 50° C.; in another example, a probe and target sequence hybridize in 7% SDS, 0.5 M NaPO₄, 1 mm EDTA, 1% BSA at 50° C. followed by washing in 1×SSC, 0.1% SDS at 50° C.; in another example, a probe and target sequence hybridize in 7% SDS, 0.5 M NaPO₄, 1 mm EDTA, 1% BSA at 50° C. followed by washing in 0.5×SSC, 0.1% SDS at 50° C.; in another example, a probe and target sequence hybridize in 7% SDS, 0.5 M NaPO₄, 1 mm EDTA, 1% BSA at 50° C. followed bywashing in 0.1×SSC, 0.1% SDS at 50° C.; in yet another example, a probe and target sequence hybridize in 7% SDS, 0.5 M NaPO₄, 1 mm EDTA, 1% BSA at 50° C. followed by washing in 0.1×SSC, 0.1% SDS at 65° C. In some embodiments, hybridization conditions comprise hybridization in a roller tube for at least 12 hours at 42° C. In each of the above conditions, the sodium phosphate hybridization buffer can be replaced by a hybridization buffer comprising 6×SSC (or 6×SSPE), 5×Denhardt's reagent, 0.5% SDS, and 100 g/ml carrier DNA, including 0-50% formamide, with hybridization and wash temperatures chosen based upon the desired stringency. Other hybridization and wash conditions are known to those of skill in the art (see also Sambrook and Russell, 2001; Ausubel et al., 2002; and Ausubel et al., 2003; each of which is incorporated herein in its entirety). As is known in the art, the addition of formamide in the hybridization solution reduces the Tₘ by about 0.4° C. Thus, high stringency conditions include the use of any of the above solutions and 0% formamide at 65° C., or any of the above solutions plus 50% formamide at 42° C.

For some high-density glass-based microarray experiments, hybridization at 65° C. is too stringent for typical use, at least in part because the presence of fluorescent labels destabilizes the nucleic acid duplexes (Randolph & Waggoner, 1997). Alternatively, hybridization can be performed in a formamide-based hybridization buffer as described in Piétu et al., 1996.

A microarray format can be selected for use based on its suitability for electrochemical-enhanced hybridization. Provision of an electric current to the microarray, or to one or more discrete positions on the microarray facilitates localization of a target nucleic acid sample near probes immobilized on the microarray surface. Concentration of target nucleic acid near arrayed probe accelerates hybridization of a nucleic acid of the sample to a probe. Further, electronic stringency control allows the removal of unbound and nonspecifically bound DNA after hybridization. See U.S. Pat. Nos. 6,017,696 to Heller and 6,245,508 to Heller and Sosnowski.

II.E.5.iii. Hybridization in Solution

In some embodiments of the presently disclosed subject matter, an amplified and/or labeled nucleic acid sample is hybridized to one or more probes in solution. Representative stringent hybridization conditions for complementary nucleic acids having more than about 100 complementary residues are overnight hybridization in 50% formamide with 1 mg of heparin at 42° C. An example of highly stringent wash conditions is 15 minutes in 0.1×SSC, 5 M NaCl at 65° C. An example of stringent wash conditions is 15 minutes in 0.2×SSC buffer at 65° C. (see Sambrook and Russell, 2001, for a description of SSC buffer). A high stringency wash can be preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4-6×SSC at 40° C. Stringent conditions can also be achieved with the addition of destabilizing agents such as formamide.

For short probes (e.g., about 10 to 50 nucleotides), stringent conditions typically involve salt concentrations of less than about 1M Na⁺ ion, typically about 0.01 M to 1 M Na⁺ ion concentration (or other salts) at pH 7.0-8.3, and the temperature is typically at least about 30° C.

Optionally, nucleic acid duplexes or hybrids can be captured from the solution for subsequent analysis, including detection assays. For example, in a simple assay, a single pathogen-specific probe set is hybridized to an amplified and labeled RNA sample derived from a target nucleic acid sample. Following hybridization, an antibody that recognizes DNA:RNA hybrids is used to precipitate the hybrids for subsequent analysis. The presence of the pathogen is determined by detection of the label in the precipitate.

Alternate capture techniques can be used as will be understood to one of skill in the art, for example, purification by a metal affinity column when using probes comprising a histidine tag. As another example, the hybridized sample can be hydrolyzed by alkaline treatment wherein the double-stranded hybrids are protected while non-hybridizing single-stranded template and excess probe are hydrolyzed. The hybrids are then collected using any nucleic acid purification technique for further analysis.

To assess the expression of multiple genes and/or samples from multiple different sources simultaneously, probes or probe sets can be distinguished by differential labeling of probes or probe sets. Alternatively, probes or probe sets can be spatially separated in different hybridization vessels.

In some embodiments, a probe or probe set having a unique label is prepared for each gene or source to be detected. For example, a first probe or probe set can be labeled with a first fluorescent label, and a second probe or probe set can be labeled with a second fluorescent label. Multi-labeling experiments should consider label characteristics and detection techniques to optimize detection of each label. Representative first and second fluorescent labels are Cy3 and Cy5 (Amersham Pharmacia Biotech of Piscataway, New Jersey, United States of America), which can be analyzed with good contrast and minimal signal leakage.

A unique label for each probe or probe set can further comprise a labeled microsphere to which a probe or probe set is attached. A representative system is LabMAP (Luminex Corporation of Austin, Tex., United States of America). Briefly, LabMAP (Laboratory Multiple Analyte Profiling) technology involves performing molecular reactions, including hybridization reactions, on the surface of color-coded microscopic beads called microspheres. When used in accordance with the methods of the presently disclosed subject matter, an individual pathogen-specific probe or probe set is attached to beads having a single color-code such that they can be identified throughout the assay. Successful hybridization is measured using a detectable label of the amplified nucleic acid sample, wherein the detectable label can be distinguished from each color-code used to identify individual microspheres. Following hybridization of the randomly amplified, labeled nucleic acid sample with a set of microspheres comprising pathogen-specific probe sets, the hybridization mixture is analyzed to detect the signal of the color-code as well as the label of a sample nucleic acid bound to the microsphere. See Vignali 2000; Smith et al., 1998; and PCT International Patent Application Publication Nos. WO 01/13120; WO 01/14589; WO 99/19515; WO 99/32660; and WO 97/14028.

II.E.6. Detection

Methods for detecting hybridization are typically selected according to the label employed.

In the case of a radioactive label (e.g., ³²P-dNTP) detection can be accomplished by autoradiography or by using a phosphorimager as is known to one of skill in the art. In some embodiments, a detection method can be automated and is adapted for simultaneous detection of numerous samples.

Common research equipment has been developed to perform high-throughput fluorescence detecting, including instruments from GSI Lumonics (Watertown, Mass., United States of America), Amersham Pharmacia Biotech/Molecular Dynamics (Sunnyvale, Calif., United States of America), Applied Precision Inc. (Issauah, Wash., United States of America), Genomic Solutions Inc. (Ann Arbor, Mich., United States of America), Genetic MicroSystems Inc. (Woburn, Mass., United States of America), Axon (Foster City, Calif., United States of America), Hewlett Packard (Palo Alto, Calif., United States of America), and Virtek (Woburn, Mass., United States of America). Most of the commercial systems use some form of scanning technology with photomultiplier tube detection. Criteria for consideration when analyzing fluorescent samples are summarized by Alexay et al., 1996.

In some embodiments, a nucleic acid sample or probe is labeled with far infrared, near infrared, or infrared fluorescent dyes. Following hybridization, the mixture of nucleic acids and probes is scanned photoelectrically with a laser diode and a sensor, wherein the laser scans with scanning light at a wavelength within the absorbance spectrum of the fluorescent label, and light is sensed at the emission wavelength of the label. See U.S. Pat. Nos. 6,086,737 to Patonay et al.; 5,571,388 to Patonav et al.; 5,346,603 to Middendorf & Brumbaugh; 5,534,125 to Middendorf et al.; 5,360,523 to Middendorf et al.; 5,230,781 to Middendorf & Patonay; 5,207,880 to Middendorf & Brumbaugh; and 4,729,947 to Middendorf & Brumbaugh. An ODYSSEY™ infrared imaging system (Li-Cor, Inc. of Lincoln, Nebr., United States of America) can be used for data collection and analysis.

If an epitope label has been used, a protein or compound that binds the epitope can be used to detect the epitope. For example, an enzyme-linked protein can be subsequently detected by development of a calorimetric or luminescent reaction product that is measurable using a spectrophotometer or luminometer, respectively.

In some embodiments, INVADER® technology (Third Wave Technologies of Madison, Wis., United States of America) is used to detect target nucleic acid/probe complexes. Briefly, a nucleic acid cleavage site (such as that recognized by a variety of enzymes having 5′ nuclease activity) is created on a target sequence, and the target sequence is cleaved in a site-specific manner, thereby indicating the presence of specific nucleic acid sequences or specific variations thereof. See U.S. Pat. Nos. 5,846,717 to Brow et al.; 5,985,557 to Prudent et al.; 5,994,069 to Hall et al.; 6,001,567 to Brow et al.; and 6,090,543 to Prudent et al.

In some embodiments, target nucleic acid/probe complexes are detected using an amplifying molecule, for example a poly-dA oligonucleotide as described by Lisle et al., 2001. Briefly, a tethered probe is employed against a target nucleic acid having a complementary nucleotide sequence. A target nucleic acid having a poly-dT sequence, which can be added to any nucleic acid sequence using methods known to one of skill in the art, hybridizes with an amplifying molecule comprising a poly-dA oligonucleotide. Short oligo-dT₄₀ signaling moieties are labeled with any suitable label (e.g., fluorescent, chemiluminescent, radioisotopic labels). The short oligo-dT₄₀ signaling moieties are subsequently hybridized along the molecule, and the label is detected.

The presently disclosed subject matter also envisions use of electrochemical technology for detecting a nucleic acid hybrid according to the disclosed method. In this case, the detection method relies on the inherent properties of DNA, and thus a detectable label on the target sample or the probe/probe set is not required. In some embodiments, probe-coupled electrodes are multiplexed to simultaneously detect multiple genes using any suitable microarray or multiplexed liquid hybridization format. To enable detection, gene-specific and control probes are synthesized with substitution of the non-physiological nucleic acid base inosine for guanine, and subsequently coupled to an electrode. Following hybridization of a nucleic acid sample with probe-coupled electrodes, a soluble redox-active mediator (e.g., ruthenium 2,2′-bipyridine) is added, and a potential is applied to the sample. In the absence of guanine, each mediator is oxidized only once. However, when a guanine-containing nucleic acid is present, by virtue of hybridization of a sample nucleic acid molecule to the probe, a catalytic cycle is created that results in the oxidation of guanine and a measurable current enhancement. See U.S. Pat. Nos. 6,127,127 to Eckhardt et al.; 5,968,745 to Thorp et al.; and 5,871,918 to Thorp et al.

Surface plasmon resonance spectroscopy can also be used to detect hybridization. See e.g., Heaton et al., 2001; Nelson et al., 2001; and Guedon et al., 2000.

II.E.7. Data Analysis

Databases and software designed for use with use with microarrays is discussed in U.S. Pat. No. 6,229,911 to Balaban & Aggarwal, a computer-implemented method for managing information, stored as indexed tables, collected from small or large numbers of microarrays, and U.S. Pat. No. 6,185,561 to Balaban & Khurgin, a computer-based method with data mining capability for collecting gene expression level data, adding additional attributes and reformatting the data to produce answers to various queries. U.S. Pat. No. 5,974,164 to Chee, disclose a software-based method for identifying mutations in a nucleic acid sequence based on differences in probe fluorescence intensities between wild type and mutant sequences that hybridize to reference sequences.

Analysis of microarray data can also be performed using the method disclosed in Tusher et al., 2001, which describes the Significance Analysis of Microarrays (SAM) method for determining significant differences in gene expression among two or more samples.

II.F. Profiles

Once an expression level is determined for a gene, a profile can be created. As used herein, the term “profile” (e.g., a “gene expression profile”) refers to a repository of the expression level data that can be used to compare the expression levels of different genes among various subjects. For example, for a given subject, the term “profile” can encompass the expression levels of all genes detected in whatever units (as described herein above) are chosen.

The term “profile” is also intended to encompass manipulations of the expression level data derived from a subject. For example, once relative expression levels are determined for a given set of genes in a subject, the relative expression levels for that subject can be compared to a standard to determine if the expression levels in that subject are higher or lower than for the same genes in the standard. Standards can include any data deemed to be relevant for comparison.

In some embodiments, a standard is prepared by determining the average expression level of a gene in a normal population, a normal population being defined as subjects that do not have connective tissue disease and/or injury. In some embodiments, a standard is prepared by determining the average expression level of a gene in a population of subjects that do have a connective tissue disease and/or injury. In some embodiments, a standard is prepared by determining the average expression level of a gene in the population as a whole (i.e. subjects are grouped together irrespective of connective tissue disease and/or injury status). In some embodiments, a standard is prepared by determining the average expression level of a gene in a normal population, the average expression level of a gene in an population of subjects with connective tissue disease and/or injury, adding those two values, and dividing the sum by two to determine the midpoint of the average expression in these populations. In this latter embodiment, a profile for a “new” subject can be compared to the standard, and the profile can further comprise data indicating whether for each gene, the expression level in the new subject is higher or lower than the expression level of that gene in the standard.

For example, a new subject's profile can comprise a score of “1” for each gene for which the expression in the subject is higher than in the standard, and a score of “0” for each gene for which the expression in the subject is lower than in the standard. In this way, a profile can comprise an overall “score”, the score being defined as the sum total of all the ones and zeroes present in the profile. These scores can then be used to in the methods disclosed herein to diagnose, detect the progression of, and/or monitor a treatment in the new subject. It is understood that the use of 1s and 0s is exemplary only, and any convenient value can be assigned in the practice of the methods of the presently claimed subject matter.

### III. KITS

The presently disclosed subject matter further includes kits comprising, in different combinations, high-density oligonucleotide arrays and reagents for use with the arrays. The kits can be used, for example, to predict or model the toxic response of a test compound, to monitor the progression of disease states, to identify genes that show promise as new drug targets, and to screen known and newly designed drugs as potential therapeutics.

In some embodiments, a kit comprises a plurality of reagents that can be used to detect expression levels for one or more (e.g., 1, 2, 3, 4, 5, 6, 7, 8, 9, 10, 15, 20, 25, 50, or more) of genes disclosed herein, such as in Tables 1-4. For example, a kit comprises a plurality of reagents that can be used to detect expression levels for in some embodiments at least five and in some embodiments at least 10 of genes disclosed herein, such as in Tables 1-4. In some embodiments, the plurality of reagents comprise one or more (e.g., 1, 5, 10, or more) oligonucleotide pairs, each pair of which can be employed to specifically amplify one of the genes listed herein, such as in Tables 1-4. In some embodiments, a kit comprises an array comprising one or more oligonucleotides attached thereto that specifically binds to a gene product (e.g., an RNA or a cDNA derived therefrom) from one or more of the genes listed herein, such as in Tables 1-4. In some embodiments, the solid support comprises one or more oligonucleotides that specifically binds to a product of a control gene and/or the kit comprises at least one oligonucleotide pair that can be employed to specifically amplify a product from a control gene, wherein the phrase “control gene” refers to a gene the expression of which is known or suspected of not being differentially expressed in the samples being analyzed. Representative control genes include the so-called “housekeeping genes”, a listing of which is disclosed in Su et al., 2003 (19 Trends in Genetics 362-365), incorporated herein by reference in its entirety.

The kits can be employed in the pharmaceutical industry, where the need for early drug testing is strong due to the high costs associated with drug development, but where bioinformatics, in particular gene expression informatics related to tendon cells, is still lacking. These kits will reduce the costs, time and risks associated with traditional new drug screening using cell cultures and laboratory animals. The results of large-scale drug screening of pre-grouped patient populations, pharmacogenomics testing, can also be applied to select drugs with greater efficacy and fewer side-effects. The kits can also be used by smaller biotechnology companies and research institutes that do not have the facilities for performing such large-scale testing themselves.

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### General Materials and Methods for Examples 1-4

Production and Labeling of cDNA. RNA was purified using Qiagen columns (Qiagen Inc., Valencia, Calif., United States of America). RNA was eluted with water and stored in ethanol at −80° C. Samples were reconstituted in water and the quality of the RNA checked by separation in an acrylamide gel with a ratio comparison of 18 to 28S rRNA bands (acceptable RNA preparations had a 28S:18S intensity ratio of at least about 2:1).

RNA was then prepared for a reverse transcriptase reaction using random hexamers to prepare cDNAs. A first sample of RNAs from one tissue or cell type was reverse transcribed into cDNAs using dCTP labeled with Cyanine 3 (a green dye fluorophore; Cy3) as the control dye while a second sample of RNAs from a second tissue or cell type was reverse transcribed using dCTP labeled with cyanine 5 (a red dye fluorophore; Cy5).

Hybridization of Samples to Microarrays. cDNAs from the first sample or the second sample were pooled in equal proportions then hybridized with arrayed DNA sequences. Arrays that were employed were the Agilent Whole Mouse Genome Oligo Microarray Kit (Product No. G4122A; Agilent Technologies, Inc., Palo Alto, Calif., United States of America) for mouse cells and tissues, and a microarray produced by the University of North Carolina at Chapel Hill's Microarray Database Facility. ARRAYASSIST® software (available from Stratagene, La Jolla, Calif., United States of America) was used for expression analysis. The hybridizations and washes were performed according to the procedures disclosed in the Agilent Technologies, Inc. “Two-Color Microarray Based Gene Expression Analysis” Manual.

Hybridized arrays were then imaged and fluorescence quantitation was made for each dye and each spot according to the Agilent Technologies, Inc. “Two-Color Microarray Based Gene Expression Analysis” Manual. The ratio of fluorescence intensities for red and green for each spot was proportional to the relative abundance of each cDNA in the target specimens.

Statistical Analysis. The Significance Analysis of Microarrays (SAM) method of Tusher et al., 2001 was employed for determining significant differences in gene expression among two or more samples.

### Example 1

### Comparisons of the Tendon and Muscle Transcriptomes

Gastrocnemious muscle and Achilles tendon tissues were collected at their anatomic midpoints with separate sterile instruments and pooled from 6 wild type (wt) mice (E129 genetic background) weighing 26 g and immediately frozen in liquid N₂. Tissues were thawed and mechanically homogenized in TRIZOL® (Invitrogen Corporation, Carlsbad, Calif., United States of America). Nucleic acids were extracted, precipitated, and the samples subjected to DNase treatment. RNA was purified using Qiagen columns (Qiagen Inc., Valencia, Calif., United States of America).

RNA was isolated and reverse transcribed as described above in General Materials and Methods. Mouse Achilles tendon (AT) RNAs were reverse transcribed into cDNAs labeled with Cyanine 3 (a green dye fluorophore; Cy3) as the control dye while gastrocnemius muscle (GM) RNAs were labeled with cyanine 5 (a red dye fluorophore; Cy5). cDNAs from AT or GM were pooled in equal proportions then hybridized with arrayed DNA sequences using the Agilent chip. Hybridized arrays were then imaged and fluorescence quantitation was made for each dye and each spot.

Approximately 41,000 genes were assessed with the Agilent Whole Mouse Genome Oligo Microarray Kit (Product No. G4122A; Agilent Technologies, Inc., Palo Alto, Calif., United States of America) comparing tendon and muscle expression levels that were graded as positive. The data presented in Table 1 show the genes expressed for which at least a 4-fold difference in expression level was observed between tendon and muscle. For instance, given a minimum of a 4-fold difference in gene expression as a baseline to determine differences, about 100 genes were expressed more in tendon than muscle, nineteen at 8 fold, and seven at 16 fold. ARRAYASSIST® software (available from Stratagene, La Jolla, Calif., United States of America) was used for expression analysis. Of these seven genes that had an expression level that differed at least 16 fold between tendon and muscle, five of had names attributed to them by the microarray manufacturer.

Surprisingly, genes that were most highly expressed in tendon compared to muscle were loricrin and other keratins. Other highly expressed genes included a several procollagens, fibronectin 1, secreted phosphoprotein 1 (Sppl), several cartilage-related genes (e.g., cartilage intermediate layer protein 2 (Cilp2) and cartilage oligomeric matrix protein (Comp)), and proteoglycan 4, among others.

TABLE 1
Comparison of Gene Expression Levels Between Wild Type Mouse Gastrocnemius Muscle and Achilles Tendon^{a}

| | | | | | | | |
|---|---|---|---|---|---|---|---|
| | | SEQ | Experi- | Experiment | Experiment | | |
| Agilent ID No. | NAME^{b} | ID NO. | ment A | B | C | Mean | STDEV |

A. Genes More Highly Expressed by at Least Two-fold in Gastrocnemius Muscle than Achilles Tendon

| | | | | | | | |
|---|---|---|---|---|---|---|---|
| A_51_P199168 | Cell death-inducing DNA fragmentation factor, alpha subunit- | 1 | 4.3150 | 4.6950 | −1.8540 | 2.3853 | 3.6763 |
| | like effector A Cidea NM_007702 | | | | | | |
| A_51_P194099 | Thyroid hormone responsive SPOT14 homolog (Rattus) Thrsp | 2 | 2.8080 | 3.6780 | 0.3690 | 2.2850 | 1.7154 |
| | NM_009381 | | | | | | |
| A_52_P347176 | cDNA sequence BC034068 BC034068 | 3 | 3.0830 | 2.8980 | 0.6060 | 2.1957 | 1.3798 |
| A_52_P260346 | Hemoglobin, beta adult major chain Hbb-b1 NM_008220 | 4 | 1.4950 | 2.2570 | 1.7620 | 1.8380 | 0.3866 |
| A_51_P264695 | Crystallin, mu Crym NM_016669 | 5 | 1.8780 | 1.7380 | 1.4790 | 1.6983 | 0.2024 |
| A_51_P374476 | Hemoglobin, beta adult major chain Hbb-b1 NM_008220 | 4 | 1.5960 | 1.6530 | 1.7560 | 1.6683 | 0.0811 |
| A_52_P266643 | RIKEN cDNA 9630033F20 gene 9630033F20Rik NM_177003 | 6 | 1.3110 | 1.9960 | 1.6930 | 1.6667 | 0.3433 |
| A_51_P521010 | Protein phosphatase 1, regulatory (inhibitor) subunit 3C | 7 | 1.4460 | 2.0760 | 1.2470 | 1.5897 | 0.4328 |
| | Ppp1r3c NM_016854 | | | | | | |
| A_52_P208681 | Hemoglobin alpha, adult chain 1 Hba-a1 M10466 | 8 | 1.7360 | 1.5400 | 1.4890 | 1.5883 | 0.1304 |
| A_52_P346113 | Forkhead box N2 Foxn2 NM_180974 | 9 | 0.7200 | 2.1760 | 1.7990 | 1.5650 | 0.7557 |
| A_51_P233597 | Resistin Retn NM_022984 | 10 | 2.3040 | 2.1800 | 0.1880 | 1.5573 | 1.1875 |
| A_51_P137125 | Myosin binding protein H Mybph NM_016749 | 11 | 0.9340 | 1.7750 | 1.9290 | 1.5460 | 0.5356 |
| A_52_P470017 | RIKEN cDNA 2310032D16 gene 2310032D16Rik NM_028802 | 12 | 1.2160 | 1.2080 | 2.1920 | 1.5387 | 0.5658 |
| A_51_P137121 | Myosin binding protein H Mybph NM_016749 | 11 | 1.2190 | 1.8010 | 1.5860 | 1.5353 | 0.2943 |
| A_51_P464791 | RIKEN cDNA 2310032D16 gene 2310032D16Rik NM_028802 | 12 | 1.2830 | 1.1230 | 2.1860 | 1.5307 | 0.5731 |
| A_52_P320553 | TIGR Accession No. TC1515832 | 13 | 0.8790 | 2.0350 | 1.6440 | 1.5193 | 0.5880 |
| A_51_P374468 | Hemoglobin, beta adult major chain Hbb-b1 NM_008220 | 14 | 1.2810 | 1.4060 | 1.8500 | 1.5123 | 0.2990 |
| A_51_P321126 | Fatty acid synthase Fasn NM_007988 | 15 | 2.5150 | 2.4760 | −0.5110 | 1.4933 | 1.7359 |
| A_52_P492062 | ENSEMBL Accession No. ENSMUST0000000505 | 16 | 1.2920 | 1.8120 | 1.3730 | 1.4923 | 0.2798 |
| A_52_P278538 | Hemoglobin alpha, adult chain 1 Hba-a1 NM_008218 | 17 | 1.3260 | 1.7010 | 1.4400 | 1.4890 | 0.1922 |
| A_52_P467128 | RIKEN cDNA 4933434E20 gene 4933434E20Rik NM_027500 | 18 | 0.5820 | 1.8600 | 2.0160 | 1.4860 | 0.7868 |
| A_51_P250217 | Phosphoenolpyruvate carboxykinase 1, cytosolic Pck1 | 19 | 2.5840 | 2.4160 | −0.6290 | 1.4570 | 1.8085 |
| | NM_011044 | | | | | | |
| A_52_P82991 | ENSEMBL Accession No. ENSMUST00000050537 | 20 | 1.0240 | 1.6640 | 1.6380 | 1.4420 | 0.3622 |
| A_52_P602147 | Myosin, heavy polypeptide 4, skeletal muscle Myh4 | 21 | 0.9550 | 1.7160 | 1.6240 | 1.4317 | 0.4154 |
| | NM_010855 | | | | | | |
| A_52_P344376 | Eukaryotic translation initiation factor 4A2 Eif4a2 NM_013506 | 22 | 0.8890 | 1.4970 | 1.8830 | 1.4230 | 0.5011 |
| A_51_P489452 | Cysteine dioxygenase 1, cytosolic Cdo1 NM_033037 | 23 | 2.3810 | 2.5530 | −0.6910 | 1.4143 | 1.8253 |
| A_51_P267986 | Cytosolic ovarian carcinoma antigen 1 Cova1 NM_145951 | 24 | 1.0730 | 1.4080 | 1.7550 | 1.4120 | 0.3410 |
| A_52_P127682 | Neural stem cell-derived dendrite regulator Nsddr AK129183 | 25 | 0.9560 | 1.1200 | 2.1500 | 1.4087 | 0.6472 |
| A_52_P654534 | Orthodenticle homolog 3 (Drosophila) Otx3 NM_130865 | 26 | 0.9850 | 1.2920 | 1.8440 | 1.3737 | 0.4353 |
| A_52_P323044 | High mobility group box 1 Hmgb1 NM_010439 | 27 | 0.9220 | 1.6120 | 1.5860 | 1.3733 | 0.3911 |
| A_52_P317346 | RIKEN cDNA D330025O06 gene D330025O06Rik AK084656 | 28 | 0.9550 | 1.5810 | 1.4920 | 1.3427 | 0.3387 |
| A_52_P679105 | Protease, serine, 23 Prss23 NM_029614 | 29 | 0.9840 | 0.8970 | 2.1310 | 1.3373 | 0.6887 |
| A_52_P655842 | Ankyrin 1, erythroid Ank1 NM_031158 | 30 | 0.6760 | 1.8460 | 1.4800 | 1.3340 | 0.5985 |
| A_52_P475825 | RIKEN cDNA 1110032D12 gene 1110032D12Rik NM_019770 | 31 | 0.4690 | 1.7280 | 1.8030 | 1.3333 | 0.7495 |
| A_52_P513347 | Phosphorylase kinase beta Phkb NM_199446 | 32 | 1.3770 | 1.4420 | 1.1720 | 1.3303 | 0.1409 |
| A_52_P5420 | Mitochondrial ribosomal protein S23 Mrps23 NM_024174 | 33 | 1.3630 | 1.2520 | 1.3590 | 1.3247 | 0.0630 |
| A_51_P235835 | RIKEN cDNA 2310061N23 gene D12Ertd647e AK075797 | 34 | 1.4040 | 1.8450 | 0.6950 | 1.3147 | 0.5802 |
| A_51_P114094 | Calsyntenin 3 Clstn3 NM_153508 | 35 | 2.3000 | 2.2920 | −0.6690 | 1.3077 | 1.7118 |
| A_52_P484807 | S-adenosylmethionine decarboxylase 1 Amd1 NM_009665 | 36 | 0.9650 | 1.0000 | 1.9360 | 1.3003 | 0.5508 |
| A_52_P224104 | Calmodulin 1 Calm1 NM_009790 | 37 | 0.5590 | 2.1990 | 1.1410 | 1.2997 | 0.8314 |
| A_52_P213909 | Hemoglobin, beta adult major chain Hbb-b1 NM_008220 | 14 | 1.1520 | 1.0120 | 1.7280 | 1.2973 | 0.3795 |
| A_52_P48569 | Solute carrier family 38, member 4 Slc38a4 NM_027052 | 38 | 1.1690 | 0.9600 | 1.7580 | 1.2957 | 0.4138 |
| A_51_P307624 | Phosphorylase kinase beta Phkb NM_199446 | 32 | 1.1800 | 1.5600 | 1.0870 | 1.2757 | 0.2506 |
| A_51_P198045 | RAB28, member RAS oncogene family Rab28 AK012286 | 39 | 0.4530 | 2.2100 | 1.1630 | 1.2753 | 0.8839 |
| A_52_P568895 | Potassium voltage-gated channel, shaker-related subfamily, | 40 | 0.8690 | 0.8550 | 2.0960 | 1.2733 | 0.7125 |
| | beta member 1 Kcnab1 NM_010597 | | | | | | |
| A_52_P101454 | Cardiomyopathy associated 5 Cmya5 AJ575748 | 41 | 0.4850 | 1.8890 | 1.4320 | 1.2687 | 0.7161 |
| A_52_P34806 | Karyopherin (importin) alpha 3 Kpna3 NM_008466 | 42 | 1.0010 | 1.0220 | 1.7770 | 1.2667 | 0.4421 |
| A_51_P452779 | Liver glycogen phosphorylase Pygl NM_133198 | 43 | 2.3090 | 2.2190 | −0.7310 | 1.2657 | 1.7297 |
| A_52_P677822 | Transmembrane protein 5 Tmem5 NM_153059 | 44 | 1.0280 | 1.4560 | 1.2940 | 1.2593 | 0.2161 |
| A_52_P89683 | Similar to L-lactate dehydrogenase A chain (LDH-A) (LDH | 45 | 0.6710 | 1.6600 | 1.4390 | 1.2567 | 0.5191 |
| | muscle subunit) (LDH-M) XM_358191 | | | | | | |
| A_51_P145404 | Tubulin, alpha 3 Tuba3 NM_009446 | 46 | 1.1400 | 1.2570 | 1.3680 | 1.2550 | 0.1140 |
| A_51_P471520 | Serine/threonine kinase 25 (yeast) Stk25 NM_021537 | 47 | 0.2820 | 1.7640 | 1.7130 | 1.2530 | 0.8413 |
| A_52_P278311 | Phosphorylase kinase alpha 1 Phka1 NM_008832 | 48 | 1.0860 | 1.6900 | 0.9790 | 1.2517 | 0.3834 |
| A_52_P411716 | Polymerase (DNA directed), eta (RAD 30 related) Polh | 49 | 0.6460 | 1.5380 | 1.5460 | 1.2433 | 0.5173 |
| | BC049159 | | | | | | |
| A_52_P55972 | Resistin Retn NM_022984 | 10 | 1.7590 | 2.2290 | −0.3020 | 1.2287 | 1.3463 |
| A_51_P338072 | Myosin, heavy polypeptide 4, skeletal muscle Myh4 | 21 | 1.0610 | 0.9400 | 1.6470 | 1.2160 | 0.3781 |
| | NM_010855 | | | | | | |
| A_52_P680710 | Karyopherin (importin) alpha 3 Kpna3 NM_008466 | 42 | 1.0320 | 1.1420 | 1.4690 | 1.2143 | 0.2273 |
| A_51_P352782 | Protein kinase C, epsilon Prkce AK017901 | 50 | 0.8870 | 1.2760 | 1.4760 | 1.2130 | 0.2995 |
| A_52_P142143 | Junctophilin 2 Jph2 BC022635 | 51 | 0.7320 | 2.2460 | 0.6580 | 1.2120 | 0.8962 |
| A_51_P519189 | Eukaryotic translation initiation factor 3, subunit 2 (beta) Eif3s2 | 52 | 0.6330 | 2.0880 | 0.9110 | 1.2107 | 0.7724 |
| | NM_018799 | | | | | | |
| A_51_P335583 | Sperm associated antigen 7 Spag7 NM_172561 | 53 | 0.4560 | 1.8730 | 1.2810 | 1.2033 | 0.7117 |
| A_51_P366672 | Solute carrier family 36 (proton/amino acid symporter), member | 54 | 2.1430 | 1.6550 | −0.1960 | 1.2007 | 1.2339 |
| | 2 Slc36a2 NM_153170 | | | | | | |
| A_51_P347862 | Actinin, alpha 1 Actn1 NM_134156 | 55 | 1.1740 | 1.7060 | 0.7130 | 1.1977 | 0.4969 |
| A_52_P480044 | Agilent Accession No. A_52_P480044 | | 0.5990 | 1.3150 | 1.6790 | 1.1977 | 0.5495 |
| A_51_P255657 | RIKEN cDNA 2210011C24 gene 2210011C24Rik AK008705 | 56 | 0.9680 | 1.0300 | 1.5160 | 1.1713 | 0.3001 |
| A_52_P16419 | Glycerol-3-phosphate dehydrogenase 1 (soluble) Gpd1 | 57 | 1.1800 | 1.3140 | 1.0000 | 1.1647 | 0.1576 |
| | NM_010271 | | | | | | |
| A_52_P171033 | RIKEN cDNA 1110007A13 gene 1110007A13Rik NM_145955 | 58 | 0.7990 | 1.8290 | 0.8570 | 1.1617 | 0.5787 |
| A_52_P402897 | Cadherin 4 Cdh4 AK049087 | 59 | 0.8290 | 1.1610 | 1.4820 | 1.1573 | 0.3265 |
| A_51_P108408 | 2,3-bisphosphoglycerate mutase Bpgm NM_007563 | 60 | 0.8580 | 1.6650 | 0.9190 | 1.1473 | 0.4493 |
| A_52_P592909 | Diacylglycerol O-acyltransferase 2 Dgat2 NM_026384 | 61 | 1.7220 | 1.8240 | −0.1070 | 1.1463 | 1.0866 |
| A_51_P436596 | Rabphilin 3A Rph3a NM_011286 | 62 | 0.8930 | 1.3310 | 1.2070 | 1.1437 | 0.2258 |
| A_52_P490032 | Ras-related GTP binding D C030003H22Rik Rragd | 63 | 0.5410 | 1.6440 | 1.2440 | 1.1430 | 0.5584 |
| | NM_027491 | | | | | | |
| A_52_P359739 | Diacylglycerol O-acyltransferase 2 Dgat2 NM_026384 | 64 | 1.5920 | 1.9140 | −0.0840 | 1.1407 | 1.0727 |
| A_52_P636038 | Parkin Park2 NM_016694 | 65 | 0.4080 | 1.8040 | 1.2020 | 1.1380 | 0.7002 |
| A_51_P143296 | Myosin, heavy polypeptide 8, skeletal muscle, perinatal Myh8 | 66 | 0.9770 | 1.3240 | 1.1070 | 1.1360 | 0.1753 |
| | NM_177369 | | | | | | |
| A_51_P380807 | Creatine kinase, muscle Ckm NM_007710 | 67 | 0.8980 | 1.4120 | 1.0960 | 1.1353 | 0.2592 |
| A_51_P116137 | Leucine-rich repeats and immunoglobulin-like domains 1 Lrig1 | 68 | 0.6260 | 1.4210 | 1.3330 | 1.1267 | 0.4358 |
| | NM_008377 | | | | | | |
| A_51_P266861 | Malic enzyme, supernatant Mod1 NM_008615 | 69 | 0.7820 | 1.0920 | 1.5060 | 1.1267 | 0.3632 |
| A_51_P225048 | Zinc finger, RAN-binding domain containing 1 Zranb1 | 70 | 0.6350 | 1.3990 | 1.3460 | 1.1267 | 0.4266 |
| | AJ250693 | | | | | | |
| A_51_P339200 | HLA-B associated transcript 5 Bat5 NM_178592 | 71 | 0.2780 | 1.8400 | 1.2550 | 1.1243 | 0.7892 |
| A_51_P499020 | Fructose bisphosphatase 2 Fbp2 NM_007994 | 72 | 1.2310 | 1.5550 | 0.5740 | 1.1200 | 0.4998 |
| A_51_P336827 | RIKEN cDNA 1810044O22 gene 1810044O22Rik NM_025558 | 73 | 1.1270 | 1.3840 | 0.8460 | 1.1190 | 0.2691 |
| A_52_P1157979 | Calmodulin 3 Calm3 NM_007590 | 74 | 0.4590 | 1.5850 | 1.2980 | 1.1140 | 0.5851 |
| A_51_P486512 | LETM1 domain containing 1 Letmd1 NM_134093 | 75 | 1.5500 | 1.5110 | 0.2770 | 1.1127 | 0.7240 |
| A_52_P2659 | ENSEMBL Accession No. ENSMUST00000059414 | 76 | 1.0250 | 1.2850 | 1.0110 | 1.1070 | 0.1543 |
| A_51_P483617 | RIKEN cDNA 0610040J01 gene 0610040J01Rik NM_029554 | 77 | 0.7220 | 0.6480 | 1.9510 | 1.1070 | 0.7319 |
| A_52_P507393 | ADP-ribosylation factor-like 10C Arl10c NM_026011 | 78 | 0.8740 | 1.8480 | 0.5910 | 1.1043 | 0.6594 |
| A_52_P436238 | Ornithine decarboxylase, structural 1 Odc1 NM_013614 | 79 | 0.5600 | 0.6020 | 2.1420 | 1.1013 | 0.9015 |
| A_52_P399054 | RIKEN cDNA 1110032D12 gene 1110032D12Rik NM_019770 | 31 | 0.3560 | 1.0230 | 1.9150 | 1.0980 | 0.7822 |
| A_52_P350554 | Potassium voltage gated channel, Shab-related subfamily, | 80 | 0.9560 | 1.5170 | 0.8170 | 1.0967 | 0.3706 |
| | member 1 Kcnb1 NM_008420 | | | | | | |
| A_52_P415047 | Olfactory receptor 973 Olfr973 NM_146613 | 81 | 0.8080 | 1.3530 | 1.1230 | 1.0947 | 0.2736 |
| A_52_P454950 | Ubiquitin-conjugating enzyme E2B, RAD6 homology | 82 | 1.0080 | 0.9170 | 1.3580 | 1.0943 | 0.2328 |
| | (S. cerevisiae) Ube2b NM_009458 | | | | | | |
| A_51_P445417 | RIKEN cDNA 4930571C24 gene 4930571C24Rik AK019803 | 83 | 1.1670 | 1.0950 | 1.0140 | 1.0920 | 0.0765 |
| A_52_P306744 | Tetraspanin 8 Tspan8 NM_146010 | 84 | 1.1430 | 0.5390 | 1.5710 | 1.0843 | 0.5185 |
| A_51_P204486 | RIKEN cDNA 1200009I06 gene 1200009I06Rik NM_028807 | 85 | 0.8900 | 1.0400 | 1.3220 | 1.0840 | 0.2193 |
| A_52_P1139966 | 10 days neonate cerebellum cDNA, RIKEN full-length enriched | 86 | 0.6760 | 1.0300 | 1.5340 | 1.0800 | 0.4312 |
| | library, clone: B930015L22 product: unknown EST, full insert | | | | | | |
| | sequence AK047066 | | | | | | |
| A_52_P315988 | RIKEN cDNA 0610010D24 gene 0610010D24Rik BC043115 | 87 | 0.7330 | 1.5520 | 0.9490 | 1.0780 | 0.4245 |
| A_51_P418765 | Selenophosphate synthetase 2 Sephs2 NM_009266 | 88 | 0.6790 | 1.0720 | 1.4750 | 1.0753 | 0.3980 |
| A_51_P364140 | Lactate dehydrogenase 1, A chain Ldh1 NM_010699 | 89 | 0.7390 | 0.7870 | 1.6960 | 1.0740 | 0.5392 |
| A_52_P151211 | Homer homolog 1 (Drosophila) Homer1 NM_152134 | 90 | 0.8850 | 1.2980 | 1.0380 | 1.0737 | 0.2088 |
| A_52_P474379 | TIGR Accession No. TC1497215 | 91 | 0.8690 | 0.7180 | 1.6340 | 1.0737 | 0.4911 |
| A_52_P409498 | Tubulin, alpha 4 Tuba4 NM_009447 | 92 | 0.4610 | 1.3710 | 1.3880 | 1.0733 | 0.5304 |
| A_52_P385606 | Creatine kinase, brain Ckb NM_021273 | 93 | 0.8160 | 1.4270 | 0.9660 | 1.0697 | 0.3184 |
| A_52_P485542 | Homeo box D8 Hoxd8 XM_355338 | 94 | 1.1010 | 1.5140 | 0.5920 | 1.0690 | 0.4618 |
| A_51_P149872 | Potassium voltage-gated channel, shaker-related subfamily, | 95 | 0.8070 | 1.4440 | 0.9520 | 1.0677 | 0.3339 |
| | member 7 Kcna7 NM_010596 | | | | | | |
| A_52_P176999 | RIKEN cDNA 9830147e 9830147 NM_177238 | 96 | 0.6980 | 1.3360 | 1.1690 | 1.0677 | 0.3309 |
| A_51_P507023 | RIKEN cDNA C630002B14 gene C630002B14Rik | 97 | 0.7080 | 0.8060 | 1.6800 | 1.0647 | 0.5351 |
| | NM_175331 | | | | | | |
| A_51_P284937 | G elongation factor Gfm1 NM_138591 | 98 | 0.4890 | 1.7460 | 0.9480 | 1.0610 | 0.6361 |
| A_51_P268559 | Isocitrate dehydrogenase 3 (NAD+) alpha Idh3a NM_029573 | 99 | 0.9290 | 1.3700 | 0.8700 | 1.0563 | 0.2732 |
| A_51_P164504 | Apolipoprotein C-I Apoc1 NM_007469 | 100 | 2.3760 | 2.1700 | −1.3870 | 1.0530 | 2.1156 |
| A_51_P450957 | Actin, alpha 2, smooth muscle, aorta Acta2 NM_007392 | 101 | 0.6080 | 0.9020 | 1.6430 | 1.0510 | 0.5333 |
| A_52_P85152 | RIKEN cDNA 5730439E10 gene 5730439E10Rik NM_175324 | 102 | 0.6090 | 1.5810 | 0.9620 | 1.0507 | 0.4920 |
| | NM_175324 | | | | | | |
| A_52_P594894 | Cell division cycle 34 homolog (S. cerevisiae) Cdc34 | 103 | 0.7030 | 0.9980 | 1.4430 | 1.0480 | 0.3725 |
| | NM_177613 | | | | | | |
| A_52_P26161 | Pentaxin related gene Ptx3 NM_008987 | 104 | 0.6870 | 1.3070 | 1.1470 | 1.0470 | 0.3219 |
| A_51_P316993 | ADP-ribosylation factor-like 6 interacting protein 2 Arl6ip2 | 105 | 0.7380 | 1.3680 | 1.0150 | 1.0403 | 0.3158 |
| | NM_019717 | | | | | | |
| A_52_P532910 | Tropomyosin 1, alpha Tpm1 NM_024427 | 106 | 1.1320 | 1.3240 | 0.6590 | 1.0383 | 0.3423 |
| A_51_P145735 | Acylphosphatase 1, erythrocyte (common) type Acyp1 | 107 | 0.8340 | 0.7740 | 1.5020 | 1.0367 | 0.4041 |
| | NM_025421 | | | | | | |
| A_52_P58024 | Similar to ALY LOC544730 XM_282933 | 108 | 0.9710 | 1.1430 | 0.9900 | 1.0347 | 0.0943 |
| A_52_P421133 | Branched chain ketoacid dehydrogenase E1, alpha polypeptide | 109 | 0.6260 | 1.2220 | 1.2540 | 1.0340 | 0.3537 |
| | Bckdha NM_007533 | | | | | | |
| A_52_P279557 | F-box protein 40 Fbxo40 AK036684 | 110 | 0.6180 | 1.8700 | 0.6060 | 1.0313 | 0.7263 |
| A_51_P445841 | DEP domain containing 6 Depdc6 NM_145470 | 111 | 0.7950 | 1.4690 | 0.8290 | 1.0310 | 0.3797 |
| A_51_P280890 | Phosphorylase kinase gamma 1 Phkg1 NM_011079 | 112 | 1.0790 | 1.1100 | 0.9040 | 1.0310 | 0.1111 |
| A_51_P411217 | Motile sperm domain containing 1 Mospd1 NM_027409 | 113 | 0.7870 | 0.8980 | 1.4010 | 1.0287 | 0.3272 |
| A_51_P283175 | ENSEMBL Accession No. ENSMUST00000021240 | 114 | 0.7120 | 0.9590 | 1.4130 | 1.0280 | 0.3556 |
| A_51_P518586 | Gene rich cluster, C2f gene Grcc2f NM_013536 | 115 | 0.9390 | 1.3510 | 0.7910 | 1.0270 | 0.2902 |
| A_52_P656699 | Actinin alpha 3 Actn3 NM_013456 | 116 | 0.6830 | 1.6910 | 0.7060 | 1.0267 | 0.5754 |
| A_51_P105927 | RAS-like, family 12 Rasl12 AK014511 | 117 | 0.9910 | 1.3210 | 0.7560 | 1.0227 | 0.2838 |
| A_51_P199187 | RIKEN cDNA 2900024C23 gene 2900024C23Rik NM_026062 | 118 | 0.9820 | 0.5260 | 1.5550 | 1.0210 | 0.5156 |
| A_51_P381763 | S-adenosylmethionine decarboxylase 1 Amd1 Z14986 | 119 | 0.6270 | 0.6130 | 1.8200 | 1.0200 | 0.6929 |
| A_51_P251717 | RIKEN cDNA 0610007e 0610007 NM_026304 | 120 | 0.7020 | 1.3140 | 1.0430 | 1.0197 | 0.3067 |
| A_52_P478339 | RIKEN cDNA 2510006C20 gene 2510006C20Rik NM_026527 | 121 | 0.6850 | 0.6940 | 1.6790 | 1.0193 | 0.5713 |
| A_51_P101879 | Peptidylprolyl isomerase D (cyclophilin D) Ppid NM_026352 | 122 | 0.7550 | 0.4180 | 1.8770 | 1.0167 | 0.7639 |
| A_51_P128575 | Secretoglobin, family 1A, member 1 (uteroglobin) Scgb1a1 | 123 | 1.2750 | 0.6140 | 1.1530 | 1.0140 | 0.3517 |
| | NM_011681 | | | | | | |
| A_52_P177021 | 6-pyruvoyl-tetrahydropterin synthase Pts NM_011220 | 124 | 0.9700 | 1.1490 | 0.9180 | 1.0123 | 0.1212 |
| A_51_P394515 | Transketolase Tkt NM_009388 | 125 | 2.3580 | 1.8760 | −1.2000 | 1.0113 | 1.9302 |
| A_51_P493886 | Glutamic pyruvate transaminase (alanine aminotransferase) 2 | 126 | 0.9710 | 1.3170 | 0.7440 | 1.0107 | 0.2886 |
| | Gpt2 NM_173866 | | | | | | |
| A_51_P203306 | Vomeronasal 1 receptor, I10 V1ri10 NM_134245 | 127 | 0.9590 | 1.0570 | 1.0160 | 1.0107 | 0.0492 |
| A_51_P389531 | Heterogeneous nuclear ribonucleoproteins methyltransferase- | 128 | 0.4980 | 1.6640 | 0.8640 | 1.0087 | 0.5963 |
| | like 2 (S. cerevisiae) Hrmt1l2 NM_019830 | | | | | | |
| A_52_P383572 | Myosin light chain, phosphorylatable, fast skeletal muscle Mylpf | 129 | 0.7980 | 1.0690 | 1.1490 | 1.0053 | 0.1840 |
| | NM_016754 | | | | | | |
| A_52_P576863 | Inosine triphosphatase (nucleoside triphosphate | 130 | 0.4850 | 1.5010 | 1.0260 | 1.0040 | 0.5084 |
| | pyrophosphatase) Itpa NM_025922 | | | | | | |
| A_51_P364146 | Lactate dehydrogenase 1, A chain Ldh1 NM_010699 | 89 | 0.7020 | 1.4990 | 0.8080 | 1.0030 | 0.4328 |

B. Genes More Highly Expressed by at Least Four Fold in Achilles Tendon than Gastrocnemius Muscle

| | | | | | | | |
|---|---|---|---|---|---|---|---|
| A_51_P196087 | Neuron navigator 1 Nav1 NM_173437 | 131 | −1.5730 | −2.3530 | −2.1010 | −2.0090 | 0.3981 |
| A_52_P173197 | Dual specificity phosphatase 7 Dusp7 NM_153459 | 132 | −4.2460 | −1.1240 | −0.6610 | −2.0103 | 1.9499 |
| A_51_P320852 | CD9 antigen Cd9 NM_007657 | 133 | −1.2400 | −2.1210 | −2.6720 | −2.0110 | 0.7223 |
| A_52_P401504 | Thrombospondin 4 Thbs4 NM_011582 | 134 | −1.9460 | −2.5530 | −1.5880 | −2.0290 | 0.4878 |
| A_51_P416647 | Kallikrein 13 Klk13 NM_010115 | 135 | −1.8420 | −1.8890 | −2.3820 | −2.0377 | 0.2991 |
| A_52_P361673 | Myosin IB Myo1b NM_010863 | 136 | −1.4800 | −2.7870 | −1.8510 | −2.0393 | 0.6735 |
| A_51_P324351 | Antigen p97 (melanoma associated) identified by monoclonal | 137 | −1.7170 | −2.3650 | −2.0390 | −2.0403 | 0.3240 |
| | antibodies 133.2 and 96.5 Mfi2 NM_013900 | | | | | | |
| A_52_P675052 | Golgi autoantigen, golgin subfamily b, macrogolgin 1 Golgb1 | 138 | −1.8130 | −2.4730 | −1.8530 | −2.0463 | 0.3700 |
| | XM_148244 | | | | | | |
| A_51_P207622 | Fibromodulin Fmod NM_021355 | 139 | −2.2310 | −1.4920 | −2.4400 | −2.0543 | 0.4981 |
| A_51_P507669 | 18S ribosomal RNA-like mRNA, partial sequence AY248756 | 140 | −1.9530 | −5.2140 | 0.9840 | −2.0610 | 3.1004 |
| A_52_P535255 | CCNDBP1 interactor Cbpin NM_026780 | 141 | −1.5590 | −2.4890 | −2.1490 | −2.0657 | 0.4706 |
| A_51_P453909 | Cytochrome P450, family 2, subfamily f, polypeptide 2 Cyp2f2 | 142 | −1.8200 | −2.1690 | −2.2140 | −2.0677 | 0.2157 |
| | NM_007817 | | | | | | |
| A_51_P133684 | Cysteine and glycine-rich protein 3 Csrp3 NM_013808 | 143 | −2.0670 | −1.9740 | −2.1820 | −2.0743 | 0.1042 |
| A_52_P626069 | Chromodomain helicase DNA binding protein 9 Chd9 | 144 | −1.7420 | −2.9350 | −1.5620 | −2.0797 | 0.7462 |
| | AK040994 | | | | | | |
| A_51_P423981 | Cathepsin S Ctss NM_021281 | 145 | −1.2570 | −1.7030 | −3.3360 | −2.0987 | 1.0945 |
| A_51_P405397 | Extracellular matrix protein 1 Ecm1 NM_007899 | 146 | −1.8490 | −1.3120 | −3.1380 | −2.0997 | 0.9385 |
| A_52_P81252 | Extracellular matrix protein 1 Ecm1 NM_172599 | 147 | −1.2990 | −3.7520 | −1.2690 | −2.1067 | 1.4250 |
| A_52_P244682 | RIKEN cDNA 5430435G22 gene 5430435G22Rik NM_145509 | 148 | −1.4250 | −2.5330 | −2.3930 | −2.1170 | 0.6034 |
| A_52_P593278 | Microtubule-associated protein 1 A Mtap1a AK018185 | 149 | −2.5310 | −1.8330 | −1.9960 | −2.1200 | 0.3651 |
| A_52_P649074 | Vacuolar protein sorting 13C (yeast) Vps13c XM_620758 | 150 | −1.6610 | −2.6560 | −2.0520 | −2.1230 | 0.5013 |
| A_51_P420276 | Plexin domain containing 2 Plxdc2 NM_026162 | 151 | −1.5640 | −2.2380 | −2.5850 | −2.1290 | 0.5192 |
| A_51_P145010 | RIKEN cDNA 2310067L16 gene AK010095 | 152 | −2.4410 | −2.1760 | −1.7820 | −2.1330 | 0.3316 |
| A_51_P204831 | Cysteine-rich protein 1 (intestinal) Crip1 NM_007763 | 153 | −1.4790 | −2.1040 | −2.8330 | −2.1387 | 0.6777 |
| A_52_P228437 | Muscleblind-like 1 (Drosophila) Mbnl1 AK088871 | 154 | −2.1620 | −3.0700 | −1.2000 | −2.1440 | 0.9351 |
| A_52_P360921 | RNA binding motif protein 5 Rbm5 NM_148930 | 155 | −1.7300 | −3.2260 | −1.4800 | −2.1453 | 0.9442 |
| A_51_P275949 | Lysyl oxidase-like 2 Loxl2 NM_033325 | 156 | −1.4840 | −2.5160 | −2.4380 | −2.1460 | 0.5746 |
| A_52_P187855 | Tripartite motif protein 37 Trim37 NM_197987 | 157 | −1.2020 | −2.4610 | −2.8260 | −2.1630 | 0.8520 |
| A_51_P244492 | Neuroblastoma, suppression of tumorigenicity 1 Nbl1 | 158 | −1.9040 | −1.9110 | −2.6790 | −2.1647 | 0.4454 |
| | NM_008675 | | | | | | |
| A_51_P462428 | UDP-N-acetyl-alpha-D-galactosamine:polypeptide N- | 159 | −1.4910 | −1.5130 | −3.5440 | −2.1827 | 1.1790 |
| | acetylgalactosaminyltransferase-like 2 Galntl2 NM_030166 | | | | | | |
| A_51_P383270 | Fraser syndrome 1 homolog (human) Fras1 NM_175473 | 160 | −1.7110 | −2.9910 | −1.8980 | −2.2000 | 0.6914 |
| A_52_P413395 | Sarcolipin Sln NM_025540 | 161 | −2.1640 | −3.0610 | −1.4090 | −2.2113 | 0.8270 |
| A_51_P504037 | SWI/SNF related, matrix associated, actin dependent regulator | 162 | −1.5960 | −3.2260 | −1.8410 | −2.2210 | 0.8789 |
| | of chromatin, subfamily a, member 2 Smarca2 NM_011416 | | | | | | |
| A_52_P10793 | Pleiotrophin Ptn NM_008973 | 163 | −1.6540 | −2.2110 | −2.8250 | −2.2300 | 0.5857 |
| A_52_P599728 | Microtubule-associated protein 1 A Mtap1a XM_194040 | 164 | −1.8590 | −3.4620 | −1.3900 | −2.2370 | 1.0865 |
| A_51_P154417 | Fibulin 1 Fbln1 NM_010180 | 165 | −1.4670 | −2.3700 | −2.8780 | −2.2383 | 0.7147 |
| A_51_P199266 | mRNA for RCK, complete cds D50494 | 166 | −1.5300 | −4.8510 | −0.3580 | −2.2463 | 2.3306 |
| A_51_P194230 | Zinc finger protein of the cerebellum 1 Zic1 NM_009573 | 167 | −1.6410 | −2.6110 | −2.5310 | −2.2610 | 0.5384 |
| A_51_P517075 | Serine (or cysteine) proteinase inhibitor, clade F, member 1 | 168 | −1.9700 | −2.2740 | −2.5970 | −2.2803 | 0.3135 |
| | Serpinf1 NM_011340 | | | | | | |
| A_51_P365344 | AHNAK nucleoprotein (desmoyokin) Ahnak NM_009643 | 169 | −1.5880 | −3.0580 | −2.2240 | −2.2900 | 0.7372 |
| A_51_P381260 | FXYD domain-containing ion transport regulator 5 Fxyd5 | 170 | −1.6930 | −2.2800 | −2.9240 | −2.2990 | 0.6157 |
| | NM_008761 | | | | | | |
| A_52_P527944 | Protein tyrosine phosphatase, receptor type Z, polypeptide 1 | 172 | −3.2350 | −5.4710 | 1.7870 | −2.3063 | 3.7170 |
| | Ptprz1 AJ428208 | | | | | | |
| A_51_P367720 | Clusterin Clu NM_013492 | 173 | −2.1010 | −2.2840 | −2.5600 | −2.3150 | 0.2311 |
| A_51_P115178 | Scavenger receptor class A, member 3 Scara3 NM_172604 | 174 | −1.9430 | −1.9610 | −3.0430 | −2.3157 | 0.6300 |
| A_51_P443902 | Kallikrein 16 Klk16 NM_008454 | 175 | −1.9360 | −2.0990 | −2.9230 | −2.3193 | 0.5291 |
| A_51_P160673 | Potassium voltage-gated channel, Isk-related family, member | 176 | −1.5110 | −1.5550 | −3.9080 | −2.3247 | 1.3714 |
| | 1-like Kcne1l NM_021487 | | | | | | |
| A_52_P508750 | Granulin Grn NM_008175 | 177 | −2.9310 | −6.2110 | 2.1580 | −2.3280 | 4.2170 |
| A_51_P353221 | Thrombospondin 4 Thbs4 NM_011582 | 134 | −1.9460 | −1.8180 | −3.2250 | −2.3297 | 0.7780 |
| A_52_P434306 | RIKEN cDNA 2310067L16 gene 2310067L16Rik XM_193814 | 178 | −2.7830 | −2.1860 | −2.0220 | −2.3303 | 0.4005 |
| A_51_P298107 | Vitrin Vit NM_028813 | 179 | −1.8720 | −1.7480 | −3.3750 | −2.3317 | 0.9057 |
| A_51_P291062 | Procollagen, type XVI, alpha 1 Col16a1 NM_028266 | 180 | −2.3150 | −1.9440 | −2.7400 | −2.3330 | 0.3983 |
| A_51_P183746 | Paired related homeobox 2 Prrx2 NM_009116 | 181 | −2.3730 | −2.8000 | −1.8920 | −2.3550 | 0.4543 |
| A_51_P395309 | Kallikrein 5 Klk5 NM_008456 | 182 | −2.1900 | −2.5250 | −2.3870 | −2.3673 | 0.1684 |
| A_52_P416123 | RIKEN cDNA 9430072K23 gene 9430072K23Rik AK020483 | 183 | −1.9190 | −4.4280 | −0.8340 | −2.3937 | 1.8434 |
| A_52_P540219 | Tissue inhibitor of metalloproteinase 2 Timp2 NM_011594 | 184 | −2.0620 | −2.5520 | −2.5790 | −2.3977 | 0.2910 |
| A_52_P440284 | RIKEN cDNA 1810057P16 gene 1810057P16Rik AK021409 | 185 | −2.0580 | −2.8640 | −2.2920 | −2.4047 | 0.4146 |
| A_52_P335089 | RIKEN cDNA 2610005L07 gene 2610005L07Rik AK009182 | 186 | −1.7860 | −3.4520 | −2.0180 | −2.4187 | 0.9024 |
| A_52_P120037 | Epithelial membrane protein 1 Emp1 NM_010128 | 187 | −1.6940 | −2.2150 | −3.3610 | −2.4233 | 0.8528 |
| A_52_P533161 | Actin-binding LIM protein 1 Ablim1 NM_178688 | 188 | −1.9010 | −3.6130 | −1.8580 | −2.4573 | 1.0011 |
| A_52_P472583 | Ribosome binding protein 1 Rrbp1 XM_622097 | 189 | −2.2550 | −2.8500 | −2.3040 | −2.4697 | 0.3303 |
| A_51_P349546 | CD109 antigen Cd109 NM_153098 | 190 | −1.7560 | −2.6680 | −2.9940 | −2.4727 | 0.6417 |
| A_51_P193475 | RIKEN cDNA D130005J21 gene C130096N06Rik NM_176841 | 191 | −2.1410 | −2.6120 | −2.6710 | −2.4747 | 0.2905 |
| A_52_P270429 | RIKEN cDNA 2200001I15 gene 2200001I15Rik NM_183278 | 192 | −1.1460 | −0.9690 | −5.3790 | −2.4980 | 2.4966 |
| A_51_P449624 | RIKEN cDNA 6430706D22 gene 6430706D22Rik BC004768 | 193 | −1.9020 | −3.7950 | −1.7990 | −2.4987 | 1.1238 |
| A_52_P434549 | Apoptotic chromatin condensation inducer 1 Acin1 NM_023190 | 194 | −1.8590 | −3.0440 | −2.5990 | −2.5007 | 0.5986 |
| A_52_P115191 | Similar to hypothetical protein 1 (rRNA external transcribed | 195 | −2.2840 | −5.4200 | 0.1530 | −2.5170 | 2.7938 |
| | spacer) - mouse LOC434481 XM_486315 | | | | | | |
| A_51_P261999 | RIKEN cDNA 2410075B13 gene 2410075B13Rik NM_146059 | 196 | −1.6930 | −3.3390 | −2.5840 | −2.5387 | 0.8239 |
| A_51_P281089 | S100 calcium binding protein A6 (calcyclin) S100a6 | 197 | −2.1130 | −2.2760 | −3.2480 | −2.5457 | 0.6137 |
| | NM_011313 | | | | | | |
| A_51_P372819 | Prostaglandin I2 (prostacyclin) synthase Ptgis NM_008968 | 198 | −2.2840 | −2.2540 | −3.1010 | −2.5463 | 0.4806 |
| A_51_P123655 | Keratocan Kera NM_008438 | 199 | −2.0950 | −3.5360 | −2.0520 | −2.5610 | 0.8446 |
| A_51_P107140 | Keratin complex 1, acidic, gene 24 Krt1-24 NM_016880 | 200 | −2.7570 | −3.2770 | −1.6570 | −2.5637 | 0.8271 |
| A_51_P249957 | Fibroblast growth factor 18 Fgf18 NM_008005 | 201 | −2.1810 | −2.8080 | −2.7390 | −2.5760 | 0.3438 |
| A_52_P581138 | DNA segment, Chr 2, ERATO Doi 485, expressed D2Ertd485e | 202 | −2.1320 | −4.0810 | −1.5190 | −2.5773 | 1.3378 |
| | NM_212450 | | | | | | |
| A_51_P157042 | Connective tissue growth factor Ctgf NM_010217 | 203 | −2.3910 | −2.5480 | −2.8380 | −2.5923 | 0.2268 |
| A_51_P334104 | Decorin Dcn NM_007833 | 204 | −2.0630 | −2.7930 | −2.9460 | −2.6007 | 0.4719 |
| A_51_P377045 | RIKEN cDNA 9430072K23 gene 9430072K23Rik AK090111 | 205 | −2.2590 | −4.3140 | −1.3510 | −2.6413 | 1.5181 |
| A_51_P416126 | Chromodomain helicase DNA binding protein 3 Chd3 | 206 | −2.5020 | −3.3810 | −2.0530 | −2.6453 | 0.6755 |
| | XM_484041 | | | | | | |
| A_52_P249402 | Prothymosin alpha Ptma NM_008972 | 207 | −1.5370 | −3.7690 | −2.6760 | −2.6607 | 1.1161 |
| A_51_P395652 | Myosin, heavy polypeptide 2, skeletal muscle, adult Myh2 | 208 | −2.9150 | −3.1360 | −1.9760 | −2.6757 | 0.6159 |
| | NM_144961 | | | | | | |
| A_51_P475049 | Ubiquitin carboxy-terminal hydrolase L1 Uchl1 NM_011670 | 209 | −2.3610 | −2.3850 | −3.3330 | −2.6930 | 0.5544 |
| A_51_P394383 | Metastasis associated lung adenocarcinoma transcript 1 (non- | 210 | −2.4460 | −3.3850 | −2.3080 | −2.7130 | 0.5860 |
| | coding RNA) Malat1 BC004722 | | | | | | |
| A_51_P321579 | Chromodomain helicase DNA binding protein 5 Chd5 | 211; | −2.3080 | −4.0960 | −1.8570 | −2.7537 | 1.1842 |
| | XM_196334; NM_029216 | 171 | | | | | |
| A_51_P314501 | Leucyl-tRNA synthetase, mitochondrial Lars2 NM_153168 | 212 | −2.2900 | −5.8750 | −0.2200 | −2.7950 | 2.8611 |
| A_51_P204153 | Insulin-like growth factor binding protein 5 Igfbp5 NM_010518 | 213 | −2.0130 | −3.5930 | −2.7890 | −2.7983 | 0.7900 |
| A_51_P412926 | Keratin complex 1, acidic, gene C29 Krt1-c29 NM_010666 | 214 | −2.2990 | −2.4740 | −3.6950 | −2.8227 | 0.7605 |
| A_52_P467690 | Spectrin beta 2 Spnb2 NM_175836 | 215 | −2.2270 | −3.5740 | −2.7160 | −2.8390 | 0.6819 |
| A_51_P110830 | A disintegrin-like and metalloprotease (reprolysin type) with | 216 | −2.4040 | −3.3270 | −2.9030 | −2.8780 | 0.4620 |
| | thrombospondin type 1 motif, 8 Adamts8 NM_013906 | | | | | | |
| A_52_P302544 | Procollagen, type VIII, alpha 2 Col8a2 NM_199473 | 217 | −2.6290 | −3.1970 | −2.8540 | −2.8933 | 0.2860 |
| A_52_P631547 | Cytokine like 1 Cyt1 BC063103 | 218 | −2.5890 | −3.1850 | −2.9130 | −2.8957 | 0.2984 |
| A_52_P496566 | AHNAK nucleoprotein (desmoyokin) Ahnak NM_175108 | 219 | −2.4980 | −3.9270 | −2.3250 | −2.9167 | 0.8792 |
| A_51_P194070 | Peptidylglycine alpha-amidating monooxygenase Pam | 220 | −2.3910 | −3.6980 | −2.6640 | −2.9177 | 0.6894 |
| | NM_013626 | | | | | | |
| A_51_P100856 | Fibronectin 1 Fn1 NM_010233 | 221 | −2.5980 | −3.2920 | −2.9870 | −2.9590 | 0.3478 |
| A_52_P846109 | Microtubule-associated protein 1 A Mtap1a XM_194040 | 164 | −1.8550 | −3.2240 | −3.9180 | −2.9990 | 1.0497 |
| A_52_P658611 | Procollagen, type I, alpha 1 Col1a1 NM_007742 | 222 | −2.0650 | −4.8650 | −2.0700 | −3.0000 | 1.6151 |
| A_51_P441898 | RIKEN cDNA 4631426H08 gene 4631426H08Rik NM_133730 | 223 | −2.7560 | −2.7210 | −3.5490 | −3.0087 | 0.4683 |
| A_51_P358765 | Secreted phosphoprotein 1 Spp1 NM_009263 | 224 | −2.4410 | −2.9060 | −4.0040 | −3.1170 | 0.8026 |
| A_52_P509020 | A disintegrin-like and metalloprotease (reprolysin type) with | 216 | −2.8000 | −3.8820 | −2.7370 | −3.1397 | 0.6437 |
| | thrombospondin type 1 motif, 8 Adamts8 NM_013906 | | | | | | |
| A_52_P525107 | Procollagen, type I, alpha 1 Col1a1 NM_007742 | 222 | −2.9910 | −3.7460 | −2.7660 | −3.1677 | 0.5133 |
| A_51_P303217 | RIKEN cDNA 1110017I16 gene 1110017I16Rik NM_026754 | 225 | −2.7180 | −3.7460 | −3.1760 | −3.2133 | 0.5150 |
| A_51_P495269 | Loricrin Lor NM_008508 | 226 | −1.1290 | −2.1470 | −6.7300 | −3.3353 | 2.9836 |
| A_51_P480073 | Chondroadherin Chad NM_007689 | 227 | −3.1360 | −3.6710 | −3.7660 | −3.5243 | 0.3396 |
| A_51_P182303 | Procollagen, type I, alpha 2 Col1a2 NM_007743 | 228 | −3.3030 | −3.6030 | −3.6930 | −3.5330 | 0.2042 |
| A_51_P207591 | Annexin A8 Anxa8 NM_013473 | 229 | −3.0530 | −3.9810 | −3.6230 | −3.5523 | 0.4680 |
| A_51_P207591 | Annexin A8 Anxa8 NM_013473 | 229 | −3.5780 | −3.2580 | −4.0470 | −3.6277 | 0.3968 |
| A_51_P486121 | AF4/FMR2 family, member 3 Aff3 AK209098 | 230 | −3.1750 | −4.8910 | −2.8890 | −3.6517 | 1.0828 |
| A_51_P207591 | Annexin A8 Anxa8 NM_013473 | 229 | −3.3790 | −3.9780 | −3.7300 | −3.6957 | 0.3010 |
| A_51_P207591 | Annexin A8 Anxa8 NM_013473 | 229 | −3.4140 | −3.9550 | −3.7250 | −3.6980 | 0.2715 |
| A_51_P220150 | FK506 binding protein 12-rapamycin associated protein 1 | 231 | −3.0980 | −3.6400 | −4.4850 | −3.7410 | 0.6990 |
| | Frap1 BC023373 | | | | | | |
| A_51_P207591 | Annexin A8 Anxa8 NM_013473 | 229 | −3.1360 | −4.3440 | −3.9180 | −3.7993 | 0.6127 |
| A_51_P105078 | S100 calcium binding protein A4 S100a4 NM_011311 | 232 | −3.0610 | −3.2290 | −5.3650 | −3.8850 | 1.2845 |
| A_51_P207591 | Annexin A8 Anxa8 NM_013473 | 229 | −3.1940 | −4.8350 | −3.6870 | −3.9053 | 0.8420 |
| A_52_P667913 | Protocadherin gamma subfamily A, 7 Pcdhga7 NM_033590 | 233 | −2.5810 | −5.4080 | −3.7700 | −3.9197 | 1.4194 |
| A_51_P512969 | Cartilage intermediate layer protein 2 Cilp2 AK004006 | 234 | −3.7500 | −4.0320 | −4.2110 | −3.9977 | 0.2324 |
| A_51_P364639 | Keratin complex 2, basic, gene 6g Krt2-6g NM_019956 | 235 | −4.5190 | −3.3910 | −4.1590 | −4.0230 | 0.5762 |
| A_51_P207591 | Annexin A8 Anxa8 NM_013473 | 229 | −3.3870 | −4.1790 | −4.5500 | −4.0387 | 0.5941 |
| A_51_P207591 | Annexin A8 Anxa8 NM_013473 | 229 | −3.4160 | −3.8430 | −4.8930 | −4.0507 | 0.7601 |
| A_51_P484526 | Wnt inhibitory factor 1 Wif1 NM_011915 | 236 | −3.4400 | −4.6040 | −4.1520 | −4.0653 | 0.5868 |
| A_51_P207591 | Annexin A8 Anxa8 NM_013473 | 229 | −3.4590 | −4.0170 | −4.7310 | −4.0690 | 0.6376 |
| A_51_P207591 | Annexin A8 Anxa8 NM_013473 | 229 | −3.4930 | −4.0270 | −4.7910 | −4.1037 | 0.6524 |
| A_52_P571290 | RIKEN cDNA 2610009E16 gene 2610009E16Rik NM_026988 | 237 | −3.0450 | −4.9320 | −4.4950 | −4.1573 | 0.9878 |
| A_51_P409010 | Cartilage oligomeric matrix protein Comp NM_016685 | 238 | −3.8980 | −4.9110 | −4.7730 | −4.5273 | 0.5494 |
| A_51_P377094 | Procollagen, type I, alpha 1 Col1a1 NM_007742 | 222 | −4.2140 | −6.4150 | −3.7230 | −4.7840 | 1.4337 |
| A_51_P404463 | RIKEN cDNA 1500015O10 gene 1500015O10Rik NM_024283 | 239 | −4.3710 | −5.6800 | −5.3680 | −5.1397 | 0.6837 |
| A_51_P280455 | Proteoglycan 4 (megakaryocyte stimulating factor, articular | 240 | −5.0520 | −6.0760 | −5.2560 | −5.4613 | 0.5420 |
| | superficial zone protein) Prg4 XM_355243 | | | | | | |

^{a}The data in the columns entitled “Experiment A”, “Experiment B”, “Experiment C”, “Mean”, and “STDEV” are presented in the form of a fold increase in gastrocnemius muscle versus Achilles tendon. The values are expressed as the log₂[fold increase]. By way of example, the first entry in Table 1A corresponds to “Cell death-inducing DNA fragmentation factor, alpha subunit-like effector A Cidea NM_007702”, and has a mean of 2.3853. Thus, this gene has expressed 2^{2.3853} (i.e., about 5.22) fold higher in gastrocnemius muscle than in Achilles tendon. In Table 1B, the means have negative values to indicate that these genes are overexpressed in Achilles tendon versus gastrocnemius nuscle (i.e., underexpressed in gastrocnemius muscle versus Achilles tendon). Therefore, Proteoglycan 4 (megakaryocyte stimulating factor, articular superficial zone protein) Prg4 XM_355243 is expressed at a level that is 2^{5.4613} (about 44.1) fold higher in Achilles tendon than in gastrocnemius muscle.
^{b}The descriptions that appear in the column headed by “NAME” include one or more of a gene description, a gene name, and one or more database accession numbers. All accession numbers are for the GENBANK ® database unless otherwise indicated. Thus, the entry “Cell death-inducing DNA fragmentation factor, alpha subunit-like effector A Cidea NM_007702”, the gene name is “Cidea”, which is “cell death-inducing DNA fragmentation factor, alpha subunit-like effector A”, and corresponds to GENBANK Accession No. NM_007702.

### Example 2

### Gene Expression Analysis of Wild Type Mouse Tendon Versus P2Y₂ Knock Out Mouse Tendon

Mice homozygous for a targeted disruption of the purinergic P2Y₂ receptor (P2Y₂-R) have been described (see Cressman et al., 1999). Achilles tendons were isolated from mice homozygous for the P2Y₂-R knockout and wild type mice as outlined in EXAMPLE 1. RNA was then prepared for a reverse transcriptase reaction using random hexamers to prepare cDNAs. Wild type mouse Achilles tendon (AT) RNAs were reverse transcribed into cDNAs labeled with Cyanine 3 (a green dye fluorophore; Cy3) as the control dye while P2Y₂-R knockout (P2Y₂ KO) tendon RNAs were labeled with cyanine 5 (a red dye fluorophore; Cy5). cDNAs from AT or P2Y₂ KO were pooled in equal proportions then hybridized with arrayed DNA sequences using the Agilent mouse microarray chip. Hybridized arrays were then imaged and fluorescence quantitation was made for each dye and each spot. The ratio of fluorescence intensities for red and green for each spot was proportional to the relative abundance of each cDNA in the target specimens. Genes that showed at least a 4 fold difference between WT and P2Y₂ KO tendon are presented in Table 2.

TABLE 2
Comparison of Gene Expression Levels Between Wild Type Mouse Achilles Tendon and P2Y2 Knockout Mouse Achilles Tendon^{a}

| | | | | | | |
|---|---|---|---|---|---|---|
| | | SEQ ID | | | | |
| CLID | NAME^{b} | NO: | Experiment A | Experiment B | Mean | STDEV |

A. Genes Upregulated at Least Three Fold in P2Y2 Knockout Mice

| | | | | | | |
|---|---|---|---|---|---|---|
| A_51_P163106 | 3-hydroxybutyrate dehydrogenase (heart, mitochondrial) Bdh | 241 | 2.8290 | 2.5870 | 2.7080 | 0.1711 |
| | NM_175177 | | | | | |
| A_51_P150145 | Adult male testis cDNA, RIKEN full-length enriched library, | 242 | 2.1000 | 2.4800 | 2.2900 | 0.2687 |
| | clone: 4932438E20 product: unknown EST, full insert sequence | | | | | |
| | AK077046 | | | | | |
| A_52_P16563 | cDNA sequence BC040823 BC040823 BC040823 | 243 | 2.3060 | 2.2440 | 2.2750 | 0.0438 |
| A_51_P480427 | Olfactory receptor 430 Olfr430 NM_146718 | 244 | 2.0470 | 2.2620 | 2.1545 | 0.1520 |
| A_52_P301724 | Ngfi-A binding protein 1 Nab1 AK018122 | 245 | 1.2280 | 2.8560 | 2.0420 | 1.1512 |
| A_51_P338443 | Angiopoietin-like 4 Angptl4 NM_020581 | 246 | 2.5020 | 1.4150 | 1.9585 | 0.7686 |
| A_51_P250217 | Phosphoenolpyruvate carboxykinase 1, cytosolic Pck1 | 19 | 1.7550 | 2.0940 | 1.9245 | 0.2397 |
| | NM_011044 | | | | | |
| A_51_P361557 | LUC7-like 2 (S. cerevisiae) Luc7l2 NM_138680 | 247 | 0.8300 | 2.8800 | 1.8550 | 1.4496 |
| A_52_P566316 | RIKEN cDNA 2310015A10 gene 2310015A10Rik AK053779 | 248 | 2.4720 | 1.2060 | 1.8390 | 0.8952 |
| A_52_P619911 | Dapper homolog 2, antagonist of beta-catenin (Xenopus) Dact2 | 249 | 2.0550 | 1.5410 | 1.7980 | 0.3635 |
| | AK041604 | | | | | |
| A_51_P324690 | Osteoclast inhibitory lectin Ocil NM_053109 | 250 | 0.5180 | 3.0320 | 1.7750 | 1.7777 |
| A_51_P400016 | RIKEN cDNA 2210407G14 gene 2210407G14Rik AK088732 | 251 | 2.6240 | 0.8960 | 1.7600 | 1.2219 |
| A_52_P274496 | Hypothetical protein 6720430O15 6720430O15 NM_183180 | 252 | 0.9730 | 2.5430 | 1.7580 | 1.1102 |
| A_51_P117666 | RIKEN cDNA 1810032O08 gene 1810032O08Rik NM_025472 | 253 | 1.8120 | 1.7010 | 1.7565 | 0.0785 |
| A_52_P779909 | Transcribed locus, strongly similar to NP_031532.2 ATP | 254 | 1.3430 | 2.1670 | 1.7550 | 0.5827 |
| | synthase, H+ transporting, mitochondrial F0 complex, subunit c | | | | | |
| | (subunit 9), isoform 1 [Mus musculus] AI481739 | | | | | |
| A_51_P364168 | Low density lipoprotein receptor-related protein 5 Lrp5 | 255 | 1.8350 | 1.6240 | 1.7295 | 0.1492 |
| | NM_008513 | | | | | |
| A_51_P166277 | Serine/arginine repetitive matrix 2 Srrm2 NM_175229 | 256 | 2.1400 | 1.2670 | 1.7035 | 0.6173 |
| A_52_P361391 | Olfactory receptor 1153 Olfr1153 NM_146640 | 257 | 1.4480 | 1.9370 | 1.6925 | 0.3458 |
| A_52_P448304 | RIKEN cDNA 2900045N06 gene 2900045N06Rik NM_028385 | 258 | 1.5500 | 1.7670 | 1.6585 | 0.1534 |
| A_51_P112627 | Sialyltransferase 7 ((alpha-N-acetylneuraminyl 2,3-beta- | 259 | 0.9360 | 2.3400 | 1.6380 | 0.9928 |
| | galactosyl-1,3)-N-acetyl galactosaminde alpha-2,6- | | | | | |
| | sialyltransferase) B Siat7b NM_009180 | | | | | |
| A_51_P483473 | Sialyltransferase 9 (CMP-NeuAc:lactosylceramide alpha-2,3- | 260 | 1.3280 | 1.9440 | 1.6360 | 0.4356 |
| | sialyltransferase) Siat9 NM_011375 | | | | | |
| A_52_P260346 | Hemoglobin, beta adult major chain Hbb-b1 NM_008220 | 4 | 2.5450 | 0.6760 | 1.6105 | 1.3216 |
| A_51_P358233 | RIKEN cDNA 2310061N23 gene 2310061N23Rik AK010014 | 261 | 1.1330 | 2.0810 | 1.6070 | 0.6703 |

B. Genes Downregulated at Least Three Fold in P2Y2 Knockout Mice

| | | | | | | |
|---|---|---|---|---|---|---|
| A_52_P174328 | RIKEN cDNA 9430063L05 gene 9430063L05Rik NM_178080 | 263 | −0.4960 | −2.7030 | −1.5995 | 1.5606 |
| A_51_P396879 | RIKEN cDNA E130201H02 gene E130201H02Rik AK021400 | 264 | −0.4340 | −2.7650 | −1.5995 | 1.6483 |
| A_51_P193336 | Nucleobindin 2 Nucb2 NM_016773 | 265 | −0.3140 | −2.8880 | −1.6010 | 1.8201 |
| A_52_P413289 | ADP-ribosylation factor-like 1 Arl1 NM_025859 | 266 | −1.1800 | −2.0230 | −1.6015 | 0.5961 |
| A_52_P652212 | Proteasome (prosome, macropain) 26S subunit, non-ATPase, | 267 | −1.0100 | −2.1930 | −1.6015 | 0.8365 |
| | 14 Psmd14 NM_021526 | | | | | |
| A_52_P581138 | DNA segment, Chr 2, ERATO Doi 485, expressed D2Ertd485e | 202 | −2.7130 | −0.4910 | −1.6020 | 1.5712 |
| | NM_212450 | | | | | |
| A_51_P239693 | Myeloid/lymphoid or mixed-lineage leukemia 5 Mll5 BC036286 | 268 | −1.2800 | −1.9250 | −1.6025 | 0.4561 |
| A_52_P214851 | Survival motor neuron domain containing 1 Smndc1 | 269 | −1.1150 | −2.0900 | −1.6025 | 0.6894 |
| | NM_172429 | | | | | |
| A_51_P296456 | RIKEN cDNA 3010027A04 gene 3010027A04Rik AK019393 | 270 | −2.2200 | −0.9890 | −1.6045 | 0.8704 |
| A_52_P228079 | Activating transcription factor 1 Atf1 NM_007497 | 271 | −1.3250 | −1.8930 | −1.6090 | 0.4016 |
| A_51_P129299 | Synaptophysin-like protein Sypl NM_013635 | 272 | −1.3800 | −1.8380 | −1.6090 | 0.3239 |
| A_52_P647740 | Kelch repeat and BTB (POZ) domain containing 10 Kbtbd10 | 273 | −0.8460 | −2.3750 | −1.6105 | 1.0812 |
| | XM_130293 | | | | | |
| A_52_P597860 | WAS protein family, member 2 Wasf2 NM_153423 | 274 | −2.2390 | −0.9890 | −1.6140 | 0.8839 |
| A_51_P409985 | RIKEN cDNA C530009C10 gene C530009C10Rik AK016794 | 275 | −1.2440 | −1.9860 | −1.6150 | 0.5247 |
| A_52_P276840 | ATPase, class II, type 9A Atp9a NM_015731 | 276 | −1.3950 | −1.8400 | −1.6175 | 0.3147 |
| A_51_P353221 | Thrombospondin 4 Thbs4 NM_011582 | 277 | −1.2460 | −1.9960 | −1.6210 | 0.5303 |
| A_52_P553841 | ATP synthase, H+ transporting mitochondrial F1 complex, beta | 278 | −0.4790 | −2.7650 | −1.6220 | 1.6164 |
| | subunit Atp5b NM_016774 | | | | | |
| A_52_P572284 | Lysosomal-associated protein transmembrane 4A Laptm4a | 279 | −1.3440 | −1.9020 | −1.6230 | 0.3946 |
| | NM_008640 | | | | | |
| A_52_P336142 | ATP-binding cassette, sub-family G (WHITE), member 2 Abcg2 | 280 | −1.5820 | −1.6730 | −1.6275 | 0.0643 |
| | NM_011920 | | | | | |
| A_51_P175146 | Copine III Cpne3 NM_027769 | 281 | −2.3250 | −0.9310 | −1.6280 | 0.9857 |
| A_51_P191400 | Titin Ttn AK035141 | 282 | −1.3860 | −1.8770 | −1.6315 | 0.3472 |
| A_52_P456279 | Chaperonin subunit 8 (theta) Cct8 NM_009840 | 283 | −1.3610 | −1.9120 | −1.6365 | 0.3896 |
| A_52_P359061 | Profilin 2 Pfn2 NM_019410 | 284 | −0.2910 | −2.9840 | −1.6375 | 1.9042 |
| A_52_P149438 | RIKEN cDNA 1110001A05 gene 1110001A05Rik NM_019809 | 285 | −1.3640 | −1.9140 | −1.6390 | 0.3889 |
| A_52_P534411 | Origin recognition complex, subunit 3-like (S. cerevisiae) Orc3l | 286 | −1.9340 | −1.3500 | −1.6420 | 0.4130 |
| | NM_015824 | | | | | |
| A_51_P470589 | Leucyl-tRNA synthetase Lars AK009823 | 287 | −2.4680 | −0.8180 | −1.6430 | 1.1667 |
| A_52_P462350 | Down-regulator of transcription 1 Dr1 NM_026106 | 288 | −2.4100 | −0.8780 | −1.6440 | 1.0833 |
| A_51_P485862 | Eukaryotic translation elongation factor 1 alpha 2 Eef1a2 | 289 | −0.7390 | −2.5590 | −1.6490 | 1.2869 |
| | NM_007906 | | | | | |
| A_51_P336491 | Casein kinase 1, alpha 1 Csnk1a1 NM_146087 | 290 | −2.1980 | −1.1030 | −1.6505 | 0.7743 |
| A_51_P106227 | Proteasome (prosome, macropain) subunit, alpha type 4 Psma4 | 291 | −1.4690 | −1.8390 | −1.6540 | 0.2616 |
| | NM_011966 | | | | | |
| A_51_P156833 | Ubiquitin specific protease 14 Usp14 NM_021522 | 292 | −2.2100 | −1.1120 | −1.6610 | 0.7764 |
| A_52_P668543 | Leukocyte-associated Ig-like receptor 1 Lair1 NM_178611 | 293 | −3.2020 | −0.1210 | −1.6615 | 2.1786 |
| A_52_P543430 | Similar to Ras-related protein Rab-2A LOC545747 XM_620188 | 294 | −1.5010 | −1.8290 | −1.6650 | 0.2319 |
| A_51_P231979 | Annexin A6 Anxa6 NM_013472 | 295 | −1.3870 | −1.9450 | −1.6660 | 0.3946 |
| A_52_P192106 | Similar to eukaryotic translation elongation factor 1 alpha 1 | 296 | −0.3710 | −2.9790 | −1.6750 | 1.8441 |
| | LOC545224 XM_619489 | | | | | |
| A_51_P421804 | Translocase of inner mitochondrial membrane 10 homolog | 297 | −2.6550 | −0.6970 | −1.6760 | 1.3845 |
| | (yeast) Timm10 NM_013896 | | | | | |
| A_52_P165455 | WW domain containing E3 ubiquitin protein ligase 1 Wwp1 | 298 | −2.3710 | −0.9820 | −1.6765 | 0.9822 |
| | BC055937 | | | | | |
| A_51_P163797 | RIKEN cDNA G630013P12 gene G630013P12Rik XM_127501 | 299 | −2.2860 | −1.0680 | −1.6770 | 0.8613 |
| A_52_P81562 | Eukaryotic translation elongation factor 2 Eef2 NM_007907 | 300 | −0.0150 | −3.3520 | −1.6835 | 2.3596 |
| A_51_P364788 | Myosin, heavy polypeptide 1, skeletal muscle, adult Myh1 | 301 | −1.1480 | −2.2280 | −1.6880 | 0.7637 |
| | AK041122 | | | | | |
| A_52_P355139 | RIKEN cDNA 1810015C04 gene 1810015C04Rik AK088619 | 302 | −1.1380 | −2.2530 | −1.6955 | 0.7884 |
| A_52_P189030 | MAX gene associated Mga NM_013720 | 303 | −1.0390 | −2.3690 | −1.7040 | 0.9405 |
| A_51_P134007 | Nucleolin Ncl NM_010880 | 304 | −1.0860 | −2.3230 | −1.7045 | 0.8747 |
| A_52_P553820 | NAP030172-1 | 305 | −2.0880 | −1.3250 | −1.7065 | 0.5395 |
| A_52_P454295 | Titin Ttn AK084780 | 306 | −2.7580 | −0.6560 | −1.7070 | 1.4863 |
| A_51_P207622 | Fibromodulin Fmod NM_021355 | 139 | −0.7320 | −2.6910 | −1.7115 | 1.3852 |
| A_52_P112188 | RIKEN cDNA A930027G11 gene Gnas NM_010309 | 307 | −0.3200 | −3.1120 | −1.7160 | 1.9742 |
| A_52_P412529 | F-box only protein 3 Fbxo3 NM_020593 | 308 | −2.4850 | −0.9530 | −1.7190 | 1.0833 |
| A_52_P657759 | Expressed sequence AI553587 AI553587 NM_178909 | 309 | −1.8270 | −1.6230 | −1.7250 | 0.1442 |
| A_52_P576886 | SMAD specific E3 ubiquitin protein ligase 2 Smurf2 | 310; 262 | −1.7610 | −1.6940 | −1.7275 | 0.0474 |
| | XM_126673; NM_025481 | | | | | |
| A_51_P229280 | Eukaryotic translation initiation factor 3, subunit 10 (theta) | 311 | −1.7190 | −1.7600 | −1.7395 | 0.0290 |
| | Eif3s10 X17373 | | | | | |
| A_52_P79187 | RIKEN cDNA 2900001A12 gene 2900001A12Rik AK013457 | 312 | −2.8400 | −0.6410 | −1.7405 | 1.5549 |
| A_51_P267544 | FSHD region gene 1 Frg1 NM_013522 | 313 | −2.2970 | −1.1850 | −1.7410 | 0.7863 |
| A_52_P134381 | Proteasome (prosome, macropain) 26S subunit, non-ATPase, | 314 | −1.4310 | −2.0660 | −1.7485 | 0.4490 |
| | 12 Psmd12 NM_025894 | | | | | |
| A_52_P496566 | RIKEN cDNA 2310047C17 gene 2310047C17Rik NM_175108 | 315 | −1.9720 | −1.5260 | −1.7490 | 0.3154 |
| A_52_P252007 | Similar to Ac2-008 LOC544824 XM_618949 | 316 | −1.0820 | −2.4210 | −1.7515 | 0.9468 |
| A_51_P450957 | Actin, alpha 2, smooth muscle, aorta Acta2 NM_007392 | 101 | −0.1270 | −3.3870 | −1.7570 | 2.3052 |
| A_52_P418477 | Tropomyosin 2, beta Tpm2 NM_009416 | 317 | −0.9320 | −2.5950 | −1.7635 | 1.1759 |
| A_51_P409988 | RIKEN cDNA C530009C10 gene C530009C10Rik NM_026577 | 318 | −1.7040 | −1.8250 | −1.7645 | 0.0856 |
| A_52_P666930 | Thyroid hormone receptor alpha Thra NM_178060 | 319 | −2.2410 | −1.2930 | −1.7670 | 0.6703 |
| A_51_P224505 | Bcl2-associated athanogene 1 Bag1 NM_009736 | 320 | −1.1760 | −2.3620 | −1.7690 | 0.8386 |
| A_51_P387670 | GTP binding protein 4 Gtpbp4 NM_027000 | 321 | −2.1130 | −1.4590 | −1.7860 | 0.4624 |
| A_51_P160870 | Reticulon 4 Rtn4 NM_194054 | 322 | −1.6000 | −1.9730 | −1.7865 | 0.2638 |
| A_51_P257762 | RIKEN cDNA A930006P13 gene Pcaf AK030070 | 323 | −1.2480 | −2.3430 | −1.7955 | 0.7743 |
| A_51_P343556 | Carnitine deficiency-associated gene expressed in ventricle 3 | 324 | −1.0650 | −2.5300 | −1.7975 | 1.0359 |
| | Cdv3 NM_175565 | | | | | |
| A_52_P430628 | RAB geranylgeranyl transferase, b subunit Rabggtb | 325 | −1.1370 | −2.4660 | −1.8015 | 0.9397 |
| | NM_011231 | | | | | |
| A_51_P401792 | Titin Ttn AK009648 | 282 | −1.6450 | −1.9720 | −1.8085 | 0.2312 |
| A_51_P347452 | HIV TAT specific factor 1 Htatsf1 NM_028242 | 326 | −2.2260 | −1.3920 | −1.8090 | 0.5897 |
| A_51_P259214 | Solute carrier family 39 (metal ion transporter), member 6 | 327 | −2.6540 | −0.9690 | −1.8115 | 1.1915 |
| | Slc39a6 NM_139143 | | | | | |
| A_52_P653684 | Glutamyl-prolyl-tRNA synthetase Eprs BC040802 | 328 | −1.7530 | −1.8720 | −1.8125 | 0.0841 |
| A_51_P338803 | Phosphatidylinositol glycan, class T Pigt NM_133779 | 329 | −2.4900 | −1.1420 | −1.8160 | 0.9532 |
| A_52_P508750 | Granulin Grn NM_008175 | 177 | −2.8730 | −0.7600 | −1.8165 | 1.4941 |
| A_51_P459350 | Destrin Dstn NM_019771 | 330 | −1.4080 | −2.2270 | −1.8175 | 0.5791 |
| A_52_P679966 | Sarcolemma associated protein Slmap AK129403 | 331 | −2.4530 | −1.1820 | −1.8175 | 0.8987 |
| A_52_P443846 | PTPRF interacting protein, binding protein 1 (liprin beta 1) | 332 | −2.6870 | −0.9490 | −1.8180 | 1.2290 |
| | Ppfibp1 NM_026221 | | | | | |
| A_52_P571290 | RIKEN cDNA 2610009E16 gene 2610009E16Rik BC052052 | 333 | −2.4750 | −1.1670 | −1.8210 | 0.9249 |
| A_52_P571684 | Radixin Rdx NM_009041 | 334 | −1.1540 | −2.4990 | −1.8265 | 0.9511 |
| A_52_P461517 | Ubiquitin associated protein 2-like Ubap2l NM_153489 | 335 | −2.1830 | −1.4940 | −1.8385 | 0.4872 |
| A_52_P112182 | Stimulatory G protein alpha subunit {clone WC-16} S49980 | 336 | −0.2440 | −3.4500 | −1.8470 | 2.2670 |
| A_52_P535255 | GCIP-interacting protein p29 Gcipip NM_026780 | 337 | −2.1670 | −1.5480 | −1.8575 | 0.4377 |
| A_52_P420712 | Praja 2, RING-H2 motif containing Pja2 AK122282 | 338 | −1.4070 | −2.3310 | −1.8690 | 0.6534 |
| A_52_P623337 | Nucleolin Ncl NM_010880 | 304 | −1.3390 | −2.4090 | −1.8740 | 0.7566 |
| A_51_P164030 | T-complex protein 1 Tcp1 NM_013686 | 339 | −1.1260 | −2.6220 | −1.8740 | 1.0578 |
| A_51_P198045 | RAB28, member RAS oncogene family Rab28 AK012286 | 39 | −1.9470 | −1.8230 | −1.8850 | 0.0877 |
| A_52_P472958 | RIKEN cDNA 4732497O03 gene 4732497O03Rik NM_144826 | 340 | −2.0910 | −1.6790 | −1.8850 | 0.2913 |
| A_51_P141152 | Sirtuin 1 ((silent mating type information regulation 2, homolog) | 341 | −3.0900 | −0.6830 | −1.8865 | 1.7020 |
| | 1 (S. cerevisiae) Sirt1 NM_019812 | | | | | |
| A_51_P502724 | RIKEN cDNA B430201A12 gene B430201A12Rik AK005412 | 342 | −3.2750 | −0.5250 | −1.9000 | 1.9445 |
| A_51_P479914 | Phosphatidylinositol 3-kinase, catalytic, beta polypeptide Pik3cb | 343 | −3.5070 | −0.3100 | −1.9085 | 2.2606 |
| | NM_029094 | | | | | |
| A_51_P339503 | Chaperonin subunit 4 (delta) Cct4 NM_009837 | 344 | −0.8530 | −2.9870 | −1.9200 | 1.5090 |
| A_51_P214503 | ras1 related extracellular matrix protein 2 Frem2 NM_172862 | 345 | −3.2020 | −0.6400 | −1.9210 | 1.8116 |
| A_52_P524700 | Titin Ttn AK084709 | 346 | −2.1880 | −1.6560 | −1.9220 | 0.3762 |
| A_51_P366890 | Guanosine diphosphate (GDP) dissociation inhibitor 3 Gdi3 | 347 | −1.0810 | −2.7850 | −1.9330 | 1.2049 |
| | NM_008112 | | | | | |
| A_52_P30877 | Similar to high mobility group protein BC054110 | 348 | −1.2600 | −2.6070 | −1.9335 | 0.9525 |
| A_52_P302977 | TAF9 RNA polymerase II, TATA box binding protein (TBP)- | 349 | −1.5650 | −2.3210 | −1.9430 | 0.5346 |
| | associated factor Taf9 NM_027139 | | | | | |
| A_51_P100856 | Fibronectin 1 Fn1 NM_010233 | 221 | −1.6170 | −2.2740 | −1.9455 | 0.4646 |
| A_52_P31687 | RE1-silencing transcription factor Rest NM_011263 | 350 | −2.5440 | −1.3470 | −1.9455 | 0.8464 |
| A_51_P448109 | Calpain 2 Capn2 NM_009794 | 351 | −0.8460 | −3.0730 | −1.9595 | 1.5747 |
| A_51_P320434 | Expressed sequence AI317223 AI317223 NM_001002764 | 352 | −1.9600 | −1.9610 | −1.9605 | 0.0007 |
| A_52_P358505 | RIKEN cDNA 5730485H21 gene 5730485H21Rik AK017709 | 353 | −1.4370 | −2.4880 | −1.9625 | 0.7432 |
| A_51_P391542 | Similar to proteasome alpha7/C8 subunit Psma3 NM_011184 | 354 | −1.5610 | −2.3840 | −1.9725 | 0.5819 |
| A_52_P228932 | Glycogen synthase 3, brain Gys3 NM_030678 | 355 | −2.7810 | −1.1730 | −1.9770 | 1.1370 |
| A_52_P7937 | Phosphatidic acid phosphatase 2a Ppap2a NM_008903 | 356 | −1.9400 | −2.0200 | −1.9800 | 0.0566 |
| A_52_P659477 | Titin Ttn AB100271 | 357 | −2.2450 | −1.7220 | −1.9835 | 0.3698 |
| A_52_P599317 | Heparan sulfate 6-O-sulfotransferase 2 Hs6st2 BC063327 | 358 | −3.0400 | −0.9800 | −2.0100 | 1.4566 |
| A_52_P658974 | Similar to Hmgb1 protein XM_358238 | 359 | −1.2730 | −2.7540 | −2.0135 | 1.0472 |
| A_52_P392598 | RIKEN cDNA 9430072K23 gene Ramp2 AK020134 | 360 | −3.3440 | −0.7020 | −2.0230 | 1.8682 |
| A_51_P224630 | RIKEN cDNA 1190002H09 gene 1190002H09Rik AK004450 | 361 | −1.9600 | −2.0890 | −2.0245 | 0.0912 |
| A_52_P434549 | Apoptotic chromatin condensation inducer 1 Acin1 NM_023190 | 362 | −2.3140 | −1.7750 | −2.0445 | 0.3811 |
| A_52_P615362 | Fibronectin leucine rich transmembrane protein 2 Flrt2 | 363 | −3.4150 | −0.7020 | −2.0585 | 1.9184 |
| | BC067058 | | | | | |
| A_51_P512210 | Myosin, heavy polypeptide 6, cardiac muscle, alpha Myh6 | 364 | −1.6760 | −2.4590 | −2.0675 | 0.5537 |
| | NM_010856 | | | | | |
| A_52_P464193 | Integrin-linked kinase-associated serine/threonine phosphatase | 365 | −2.0350 | −2.1080 | −2.0715 | 0.0516 |
| | 2C Ilkap NM_023343 | | | | | |
| A_52_P299231 | Solute carrier family 25 (mitochondrial carrier, phosphate | 366 | −1.2710 | −2.8770 | −2.0740 | 1.1356 |
| | carrier), member 3 Slc25a3 AK028313 | | | | | |
| A_51_P218535 | Nebulin Neb X70032 | 367 | −2.0240 | −2.1350 | −2.0795 | 0.0785 |
| A_52_P520439 | Phosphatidylethanolamine binding protein Gnaq NM_018858 | 368 | −1.7950 | −2.3710 | −2.0830 | 0.4073 |
| A_51_P486121 | AF4/FMR2 family, member 3 Aff3 AK209098 | 230 | −2.9500 | −1.2200 | −2.0850 | 1.2233 |
| A_52_P527944 | Protein tyrosine phosphatase, receptor type Z, polypeptide 1 | 172 | −3.5600 | −0.6180 | −2.0890 | 2.0803 |
| | Ptprz1 AJ428208 | | | | | |
| A_51_P161225 | DEAD box polypeptide 46 Ddx46 AK008639 | 369 | −2.3690 | −1.8110 | −2.0900 | 0.3946 |
| A_51_P247883 | Procollagen, type V, alpha 2 Col5a2 NM_007737 | 370 | −3.2350 | −0.9610 | −2.0980 | 1.6080 |
| A_52_P313185 | Synaptic vesicle glycoprotein 2 b Sv2b NM_153579 | 371 | −3.4640 | −0.7460 | −2.1050 | 1.9219 |
| A_51_P515026 | Kidney cell line derived transcript 1 Kdt1 NM_175088 | 372 | −2.2080 | −2.0850 | −2.1465 | 0.0870 |
| A_52_P644452 | Dedicator of cytokinesis 9 Dock9 AK122431 | 373 | −3.3720 | −0.9290 | −2.1505 | 1.7275 |
| A_52_P646312 | Pleckstrin homology domain containing, family A member 5 | 374 | −2.8890 | −1.4330 | −2.1610 | 1.0295 |
| | Plekha5 NM_144920 | | | | | |
| A_51_P115953 | 7 days neonate cerebellum cDNA, RIKEN full-length enriched | 375 | −1.4450 | −2.8860 | −2.1655 | 1.0189 |
| | library, clone: A730024G14 product: weakly similar to | | | | | |
| | CORTEXIN [Rattus norvegicus], full insert sequence AK042789 | | | | | |
| A_52_P630493 | DnaJ (Hsp40) homolog, subfamily B, member 6 Dnajb6 | 376 | −3.0640 | −1.2740 | −2.1690 | 1.2657 |
| | NM_011847 | | | | | |
| A_52_P379337 | Reticulon 4 Rtn4 NM_194054 | 322 | −1.9050 | −2.4400 | −2.1725 | 0.3783 |
| A_52_P675052 | Golgi autoantigen, golgin subfamily b, macrogolgin 1 Golgb1 | 138 | −2.4220 | −1.9480 | −2.1850 | 0.3352 |
| | XM_148244 | | | | | |
| A_51_P495331 | Neulin Neb AF203898 XM_130232 | 377 | −1.9880 | −2.4030 | −2.1955 | 0.2934 |
| A_52_P69998 | KDEL endoplasmic reticulum protein retention receptor 2 Kdelr2 | 378 | −3.8100 | −0.6050 | −2.2075 | 2.2663 |
| | NM_025841 | | | | | |
| A_52_P49453 | TIGR Accession No. TC1413911 | 379 | −2.7510 | −1.6770 | −2.2140 | 0.7594 |
| A_51_P108525 | Pleckstrin homology domain containing, family A | 380 | −4.0360 | −0.4190 | −2.2275 | 2.5576 |
| | (phosphoinositide binding specific) member 3 Plekha3 | | | | | |
| | NM_031256 | | | | | |
| A_52_P299505 | protein synthesis elongation factor Tu eEF-Tu, eEf-1-alpha | 381 | −0.8610 | −3.6060 | −2.2335 | 1.9410 |
| | mRNA M22432 | | | | | |
| A_51_P224534 | AHNAK nucleoprotein (desmoyokin) Ahnak NM_009643 | 169 | −1.7300 | −2.7530 | −2.2415 | 0.7234 |
| A_52_P172665 | RIKEN cDNA 4921533L14 gene 4921533L14Rik NM_026604 | 382 | −3.8720 | −0.6400 | −2.2560 | 2.2854 |
| A_52_P12877 | Heat shock protein 8 Hspa8 NM_031165 | 383 | −0.8660 | −3.6510 | −2.2585 | 1.9693 |
| A_52_P115191 | Similar to hypothetical protein 1 (rRNA external transcribed | 195 | −2.6030 | −1.9380 | −2.2705 | 0.4702 |
| | spacer) - mouse; LOC434481 XM_486315 | | | | | |
| A_51_P165504 | Twist homolog 2 (Drosophila) Twist2 NM_007855 | 384 | −4.1330 | −0.4310 | −2.2820 | 2.6177 |
| A_51_P272046 | Catenin beta Catnb NM_007614 | 385 | −1.9950 | −2.5730 | −2.2840 | 0.4087 |
| A_52_P240561 | Zinc finger protein 75 Zfp75 NM_172918 | 386 | −1.9600 | −2.6380 | −2.2990 | 0.4794 |
| A_51_P377094 | Procollagen, type I, alpha 1 Col1a1 NM_007742 | 222 | −4.0780 | −0.7130 | −2.3955 | 2.3794 |
| A_51_P186856 | Keratin complex 2, basic, gene 5 Krt2-5 NM_027011 | 387 | −0.2540 | −4.6270 | −2.4405 | 3.0922 |
| A_52_P249402 | Prothymosin alpha Ptma NM_008972 | 207 | −4.1740 | −0.8440 | −2.5090 | 2.3547 |
| A_51_P118637 | RIKEN cDNA 3110050K21 gene 3110050K21Rik AK078225 | 388 | −2.2050 | −2.9210 | −2.5630 | 0.5063 |
| A_52_P311417 | Luc7 homolog (S. cerevisiae)-like Luc7l NM_028190 | 389 | −3.8090 | −1.4190 | −2.6140 | 1.6900 |
| A_52_P421357 | Restin (Reed-Steinberg cell-expressed intermediate filament- | 390 | −3.4170 | −1.8150 | −2.6160 | 1.1328 |
| | associated protein) Rsn NM_019765 | | | | | |
| A_51_P111285 | Keratin complex 1, acidic, gene 10 Krt1-10 NM_010660 | 391 | −1.0160 | −4.4980 | −2.7570 | 2.4621 |
| A_52_P569906 | Titin Ttn AB100271 | 392 | −2.6460 | −2.9320 | −2.7890 | 0.2022 |
| A_51_P356705 | Pleckstrin homology domain containing, family B (evectins) | 393 | −4.9470 | −0.6400 | −2.7935 | 3.0455 |
| | member 2 Plekhb2 NM_145516 | | | | | |
| A_51_P395652 | Myosin, heavy polypeptide 2, skeletal muscle, adult Myh2 | 208 | −3.1670 | −2.6900 | −2.9285 | 0.3373 |
| | NM_144961 | | | | | |
| A_51_P495269 | Loricrin Lor NM_008508 | 226 | −0.4240 | −6.4160 | −3.4200 | 4.2370 |

^{a,b}The data is presented in the same fashion as described hereinabove with respect to Table 1.

### Example 3

### Gene Expression Analysis of Wild Type Mouse Tendon Versus P2Y₁/P2Y₂ Double Knock Out Mouse Tendon

Mice homozygous for a targeted disruption of the purinergic P2Y₁ receptor (P2Y₁-R) have been described (see Leon et al., 1999). Mice homozygous for the P2Y₁-R knockout (P2Y₁-R) were bred to homozygous P2Y₂-R KO mice, and mice homozygous for both the P2Y₁-R disruption and the P2Y₂-R disruption were identified (referred to herein as “double knockout” or DKO mice). DKO mice appeared to have defects in tendon development, as the tail tendon fascicle of the DKO mice was both wider (17.1 microns vs. 14.3 microns in wild type mice) and had a wavy appearance (whereas the tail tendon fascicle of the wild type mice was straight).

Achilles tendons were isolated from wild type mice and DKO mice as outlined in EXAMPLE 1. RNA was isolated and cDNAs prepared, with wild type mouse Achilles tendon (AT) RNAs reverse transcribed into cDNAs labeled with Cyanine 3 (a green dye fluorophore; Cy3) and DKO mouse tendon RNAs (DKO) labeled with cyanine 5 (a red dye fluorophore; Cy5). cDNAs from AT or DKO were pooled in equal proportions and hybridized to the Agilent mouse microarray chip. Hybridized arrays were imaged and fluorescence quantitated for each dye and each spot.

Genes that showed at least a 2 fold difference between wild type and DKO tendon are presented in Table 3. Seven genes, keratin associated protein 16-10 (Krtap 16-10; GENBANK® Accession No. NM_{—}183296), Ioricrin (Lor; GENBANK® Accession No. NM_{—}008508), keratin associated protein 6-1 (Krtap6-1; GENBANK®Accession No. NM_{—}010672), keratin complex 2, basic, gene 5 (Krt2-5; GENBANK® Accession No. NM_{—}027011), keratin associated protein 6-3 (Krtap6-3; GENBANK® Accession No. NM_{—}130866), keratin complex-1, acidic, gene C29 (Krt1-c29; GENBANK® Accession No. NM_{—}010666), and annexin A8 (Anxa8; GENBANK® Accession No. NM_{—}013473) that were upregulated in tendon versus muscle were also upregulated in DKO tendon.

TABLE 3
Comparison of Gene Expression Levels Between Wild Type Mouse Achilles Tendon and
P2Y1/P2Y2 Double Knockout Mouse Achilles Tendon^{a}

| | | | | | | | |
|---|---|---|---|---|---|---|---|
| | | SEQ ID | Experiment | | | | |
| CLID | NAME^{b} | NO: | A | Experiment B | Experiment C | Mean | STDEV |

A. Genes Upregulated at Least Two Fold in P2Y1/P2Y2 Double Knockout Mice

| | | | | | | | |
|---|---|---|---|---|---|---|---|
| A_52_P463962 | Keratin associated protein 16-10 Krtap 16-10 | 394 | 3.4660 | 3.3820 | 3.5570 | 3.4683 | 0.0875 |
| | NM_183296 | | | | | | |
| A_51_P495269 | Loricrin Lor NM_008508 | 226 | 2.9750 | 2.7400 | 2.8960 | 2.8703 | 0.1196 |
| A_51_P204350 | RIKEN cDNA 2310015J09 gene 2310015J09Rik | 395 | 2.3510 | 2.3750 | 2.6200 | 2.4487 | 0.1489 |
| | NM_027983 | | | | | | |
| A_52_P225117 | Keratin associated protein 6-1 Krtap6-1 | 396 | 2.1140 | 2.3340 | 2.5300 | 2.3260 | 0.2081 |
| | NM_010672 | | | | | | |
| A_51_P160673 | Potassium voltage-gated channel, lsk-related | 176 | 1.7330 | 2.0470 | 2.2680 | 2.0160 | 0.2688 |
| | family, member 1-like Kcne1l NM_021487 | | | | | | |
| A_52_P523368 | RIKEN cDNA 2310020A21 gene 2310020A21Rik | 397 | 2.0410 | 1.9020 | 2.0230 | 1.9887 | 0.0756 |
| | NM_175249 | | | | | | |
| A_51_P345073 | RIKEN cDNA 2310020A21 gene 2310020A21Rik | 397 | 1.8590 | 1.8780 | 2.1980 | 1.9783 | 0.1905 |
| | NM_175249 | | | | | | |
| A_52_P479051 | Keratin associated protein 6-1 Krtap6-1 | 396 | 1.9770 | 1.6440 | 2.2670 | 1.9627 | 0.3117 |
| | NM_010672 | | | | | | |
| A_51_P186856 | Keratin complex 2, basic, gene 5 Krt2-5 | 387 | 1.9690 | 1.8140 | 1.9220 | 1.9017 | 0.0795 |
| | NM_027011 | | | | | | |
| A_52_P2259 | Keratin associated protein 6-3 Krtap6-3 | 398 | 1.9030 | 1.8690 | 1.8700 | 1.8807 | 0.0193 |
| | NM_130866 | | | | | | |
| A_52_P270429 | RIKEN cDNA 2200001I15 gene 2200001I15Rik | 192 | 1.6500 | 1.8200 | 1.8180 | 1.7627 | 0.0976 |
| | NM_183278 | | | | | | |
| A_52_P437884 | Mindbomb homolog 1 (Drosophila) Mib1 | 399 | 1.9380 | 1.3670 | 1.5780 | 1.6277 | 0.2887 |
| | BC083072 | | | | | | |
| A_52_P313185 | Synaptic vesicle glycoprotein 2 b Sv2b | 371 | 1.8580 | 1.4420 | 1.4430 | 1.5810 | 0.2399 |
| | NM_153579 | | | | | | |
| A_52_P468068 | RIKEN cDNA 4732442J06 gene 4732442J06Rik | 400 | 1.3250 | 1.6060 | 1.7890 | 1.5733 | 0.2337 |
| | AV240687 | | | | | | |
| A_51_P441898 | RIKEN cDNA 4631426H08 gene 4631426H08Rik | 223 | 1.5620 | 1.5840 | 1.5530 | 1.5663 | 0.0159 |
| | NM_133730 | | | | | | |
| A_52_P22896 | SNF2 histone linker PHD RING helicase Shprh | 401 | 2.4750 | 1.3480 | 0.8700 | 1.5643 | 0.8241 |
| | AK086203 | | | | | | |
| A_52_P587738 | Purinergic receptor P2Y, G-protein coupled 2 | 402 | 1.4950 | 1.5500 | 1.6360 | 1.5603 | 0.0711 |
| | P2ry2 NM_008773 | | | | | | |
| A_51_P359046 | Secreted Ly6/Plaur domain containing 1 Slurp1 | 403 | 1.0000 | 1.5650 | 2.0230 | 1.5293 | 0.5124 |
| | NM_020519 | | | | | | |
| A_51_P412926 | Keratin complex-1, acidic, gene C29 Krt1-c29 | 404 | 1.8450 | 1.4520 | 1.2610 | 1.5193 | 0.2978 |
| | NM_010666 | | | | | | |
| A_51_P207591 | Annexin A8 Anxa8 NM_013473 | 229 | 1.3290 | 1.5270 | 1.6610 | 1.5057 | 0.1670 |
| A_51_P207591 | Annexin A8 Anxa8 NM_013473 | 229 | 1.3090 | 1.4210 | 1.7340 | 1.4880 | 0.2203 |
| A_51_P501844 | Cytochrome P450, family 26, subfamily b, | 405 | 1.6040 | 1.4840 | 1.3720 | 1.4867 | 0.1160 |
| | polypeptide 1 Cyp26b1 NM_175475 | | | | | | |
| A_51_P133684 | Cysteine and glycine-rich protein 3 Csrp3 | 143 | 1.4210 | 1.5180 | 1.4860 | 1.4750 | 0.0494 |
| | NM_013808 | | | | | | |
| A_51_P207591 | Annexin A8 Anxa8 NM_013473 | 229 | 1.3100 | 1.4640 | 1.6330 | 1.4690 | 0.1616 |
| A_51_P207591 | Annexin A8 Anxa8 NM_013473 | 229 | 1.2960 | 1.3990 | 1.6740 | 1.4563 | 0.1954 |
| A_51_P207591 | Annexin A8 Anxa8 NM_013473 | 229 | 1.1620 | 1.5230 | 1.6610 | 1.4487 | 0.2577 |
| A_51_P207591 | Annexin A8 Anxa8 NM_013473 | 229 | 1.3590 | 1.3280 | 1.6170 | 1.4347 | 0.1587 |
| A_51_P111285 | Keratin complex 1, acidic, gene 10 Krt1-10 | 391 | 1.3990 | 1.3700 | 1.5340 | 1.4343 | 0.0875 |
| | NM_010660 | | | | | | |
| A_51_P207591 | Annexin A8 Anxa8 NM_013473 | 229 | 1.1540 | 1.4410 | 1.7040 | 1.4330 | 0.2751 |
| A_51_P287635 | Purinergic receptor P2Y, G-protein coupled 2 | 402 | 1.6160 | 1.4640 | 1.2120 | 1.4307 | 0.2041 |
| | P2ry2 NM_008773 | | | | | | |
| A_51_P207591 | Annexin A8 Anxa8 NM_013473 | 229 | 1.2740 | 1.4360 | 1.5570 | 1.4223 | 0.1420 |
| A_51_P115953 | RIKEN cDNA A730024G14 gene A730024G14Rik | 406 | 1.7010 | 1.4080 | 1.1440 | 1.4177 | 0.2786 |
| | AK042789 | | | | | | |
| A_51_P232207 | Homeo box B6 Hoxb6 NM_008269 | 407 | 2.5750 | 1.0740 | 0.5770 | 1.4087 | 1.0402 |
| A_51_P207622 | Fibromodulin Fmod NM_021355 | 139 | 1.7690 | 1.2200 | 1.1880 | 1.3923 | 0.3266 |
| A_52_P573336 | Suprabasin Sbsn NM_172205 | 408 | 1.2080 | 1.2650 | 1.6620 | 1.3783 | 0.2473 |
| A_51_P267053 | Thrombospondin 3 Thbs3 NM_013691 | 409 | 1.4170 | 1.3280 | 1.3650 | 1.3700 | 0.0447 |
| A_51_P105078 | S100 calcium binding protein A4 S100a4 | 232 | 1.0120 | 1.4870 | 1.5630 | 1.3540 | 0.2986 |
| | NM_011311 | | | | | | |
| A_51_P506417 | Keratin complex 1, acidic, gene 14 Krt1-14 | 410 | 1.5020 | 1.1610 | 1.3890 | 1.3507 | 0.1737 |
| | NM_016958 | | | | | | |
| A_52_P570487 | Olfactory receptor 1344 Olfr1344 NM_177061 | 411 | 0.0180 | 3.6960 | 0.3340 | 1.3493 | 2.0384 |
| A_51_P205907 | Filamin C, gamma (actin binding protein 280) Flnc | 412 | 2.1580 | 1.1720 | 0.6700 | 1.3333 | 0.7570 |
| | XM_284175 | | | | | | |
| A_52_P686785 | P686785 Extra cellular link domain-containing 1 | 413 | 1.1530 | 1.2620 | 1.5840 | 1.3330 | 0.2241 |
| | Xlkd1 NM_053247 | | | | | | |
| A_52_P455295 | HCF-binding transcription factor Zhangfei | 414 | 1.6120 | 1.2330 | 1.1300 | 1.3250 | 0.2538 |
| | MGI: 2675296 NM_145151 | | | | | | |
| A_52_P335064 | Musculoskeletal, embryonic nuclear protein 1 | 415 | 1.4190 | 1.3810 | 1.1730 | 1.3243 | 0.1324 |
| | Mustn1 NM_181390 | | | | | | |
| A_52_P131062 | Keratin associated protein 8-1 Krtap8-1 AA739048 | 416 | 1.1430 | 1.4800 | 1.2130 | 1.2787 | 0.1778 |
| A_51_P207591 | Annexin A8 Anxa8 NM_013473 | 229 | 1.1300 | 1.2540 | 1.4270 | 1.2703 | 0.1492 |
| A_51_P364639 | Keratin complex 2, basic, gene 6g Krt2-6g | 235 | 1.2050 | 1.3140 | 1.2880 | 1.2690 | 0.0569 |
| | NM_019956 | | | | | | |
| A_51_P122321 | RIKEN cDNA 9230117N10 gene 9230117N10Rik | 417 | 1.0830 | 1.3090 | 1.3640 | 1.2520 | 0.1489 |
| | NM_133775 | | | | | | |
| A_52_P634111 | Hypothetical protein D930020L01 AK086316 | 418 | 1.4690 | 1.3450 | 0.9280 | 1.2473 | 0.2834 |
| A_51_P346445 | Heat shock protein family, member 7 | 419 | 1.3300 | 1.3720 | 0.9910 | 1.2310 | 0.2089 |
| | (cardiovascular) Hspb7 NM_013868 | | | | | | |
| A_51_P207591 | Annexin A8 Anxa8 NM_013473 | 229 | 1.0870 | 1.1070 | 1.4930 | 1.2290 | 0.2288 |
| A_51_P196844 | Oxysterol binding protein-like 3 Osbpl3 | 420 | 1.3410 | 1.0340 | 1.2720 | 1.2157 | 0.1611 |
| | NM_027881 | | | | | | |
| A_51_P346445 | Heat shock protein family, member 7 | 419 | 1.5310 | 1.1930 | 0.8980 | 1.2073 | 0.3167 |
| | (cardiovascular) Hspb7 NM_013868 | | | | | | |
| A_51_P349961 | Group specific component Gc NM_008096 | 421 | 1.9730 | 0.9430 | 0.7030 | 1.2063 | 0.6747 |
| A_52_P543684 | Kallikrein 26 Klk26 NM_010644 | 422 | 0.9810 | 1.3040 | 1.3290 | 1.2047 | 0.1941 |
| A_51_P184331 | Sodium channel, voltage-gated, type III, beta | 423 | 1.1850 | 1.0810 | 1.3270 | 1.1977 | 0.1235 |
| | Scn3b BC058636 | | | | | | |
| A_51_P313561 | Lamin A Lmna NM_019390 | 424 | 1.5420 | 0.9830 | 1.0450 | 1.1900 | 0.3064 |
| A_52_P380379 | Uncoupling protein 3, mitochondrial Ucp3 | 425 | 1.2410 | 0.9340 | 1.3750 | 1.1833 | 0.2261 |
| | NM_009464 | | | | | | |
| A_52_P592305 | Potassium voltage gated channel, Shaw-related | 426 | 1.7380 | 0.8240 | 0.9750 | 1.1790 | 0.4900 |
| | subfamily, member 1 Kcnc1 NM_008421 | | | | | | |
| A_51_P346445 | Heat shock protein family, member 7 | 419 | 1.5300 | 1.0270 | 0.9450 | 1.1673 | 0.3167 |
| | (cardiovascular) Hspb7 NM_013868 | | | | | | |
| A_52_P588483 | Fibulin 1 Fbln1 NM_010180 | 165 | 1.2090 | 1.1000 | 1.1130 | 1.1407 | 0.0595 |
| A_51_P395309 | Kallikrein 5 Klk5 NM_008456 | 182 | 0.9560 | 1.2680 | 1.1960 | 1.1400 | 0.1634 |
| A_51_P446510 | Epithelial membrane protein 3 Emp3 NM_010129 | 427 | 1.2490 | 1.0150 | 1.1270 | 1.1303 | 0.1170 |
| A_51_P505530 | Tenascin XB Tnxb NM_031176 | 428 | 1.0640 | 1.2480 | 1.0640 | 1.1253 | 0.1062 |
| A_52_P534355 | RIKEN cDNA A630042F09 A630042F09Rik | 429 | 1.1170 | 1.0610 | 1.1970 | 1.1250 | 0.0684 |
| | AK041855 | | | | | | |
| A_51_P375558 | Myocilin Myoc NM_010865 | 430 | 1.3750 | 0.9220 | 1.0750 | 1.1240 | 0.2304 |
| A_51_P346445 | Heat shock protein family, member 7 | 419 | 1.3820 | 1.0190 | 0.9690 | 1.1233 | 0.2254 |
| | (cardiovascular) Hspb7 NM_013868 | | | | | | |
| A_51_P416647 | Kallikrein 13 Klk13 NM_010115 | 135 | 0.9430 | 1.0410 | 1.3710 | 1.1183 | 0.2242 |
| A_51_P448236 | Cathepsin K Ctsk NM_007802 | 431 | 1.3690 | 0.9090 | 1.0740 | 1.1173 | 0.2330 |
| A_51_P426353 | Uncoupling protein 1, mitochondrial Ucp1 | 432 | 1.1170 | 1.2190 | 1.0010 | 1.1123 | 0.1091 |
| | NM_009463 | | | | | | |
| A_52_P213909 | Hemoglobin, beta adult major chain Hbb-b1 | 4 | 0.7730 | 1.2490 | 1.3070 | 1.1097 | 0.2930 |
| | NM_008220 | | | | | | |
| A_51_P358765 | Secreted phosphoprotein 1 Spp1 NM_009263 | 224 | 1.3240 | 0.8750 | 1.1250 | 1.1080 | 0.2250 |
| A_51_P492456 | Hyaluronan synthase1 Has1 NM_008215 | 433 | 0.9900 | 1.0620 | 1.2440 | 1.0987 | 0.1309 |
| A_51_P346445 | Heat shock protein family, member 7 | 419 | 1.3330 | 1.0840 | 0.8730 | 1.0967 | 0.2303 |
| | (cardiovascular) Hspb7 NM_013868 | | | | | | |
| A_51_P346445 | Heat shock protein family, member 7 | 419 | 1.3580 | 1.0850 | 0.8450 | 1.0960 | 0.2567 |
| | (cardiovascular) Hspb7 NM_013868 | | | | | | |
| A_51_P220150 | FK506 binding protein 12-rapamycin associated | 231 | 1.4880 | 1.0320 | 0.7660 | 1.0953 | 0.3651 |
| | protein 1 Frap1 BC023373 | | | | | | |
| A_51_P157083 | Growth arrest specific 1 Gas1 NM_008086 | 434 | 1.4550 | 0.9630 | 0.8570 | 1.0917 | 0.3191 |
| A_51_P151732 | Plakophilin 1 Pkp1 NM_019645 | 435 | 1.1290 | 1.0440 | 1.0710 | 1.0813 | 0.0434 |
| A_51_P135517 | Coagulation factor C homolog (Limulus | 436 | 0.9120 | 1.0880 | 1.2340 | 1.0780 | 0.1612 |
| | polyphemus) Coch NM_007728 | | | | | | |
| A_51_P346445 | Heat shock protein family, member 7 | 419 | 1.4930 | 1.0210 | 0.7150 | 1.0763 | 0.3919 |
| | (cardiovascular) Hspb7 NM_013868 | | | | | | |
| A_51_P475049 | Ubiquitin carboxy-terminal hydrolase L1 Uchl1 | 209 | 1.0600 | 0.9290 | 1.2240 | 1.0710 | 0.1478 |
| | NM_011670 | | | | | | |
| A_52_P218058 | C-type lectin domain family 5, member a Clec5a | 437 | 1.1100 | 1.0070 | 1.0880 | 1.0683 | 0.0542 |
| | AK046600 | | | | | | |
| A_52_P62085 | Cathepsin Z Ctsz NM_022325 | 438 | 1.4570 | 0.9500 | 0.7940 | 1.0670 | 0.3466 |
| A_51_P406328 | Serine (or cysteine) proteinase inhibitor, clade B, | 439 | 1.2910 | 1.0370 | 0.8660 | 1.0647 | 0.2138 |
| | member 6c Serpinb6c NM_148942 | | | | | | |
| A_52_P257204 | Heat shock protein 1, beta Hspcb NM_008302 | 440 | 1.5370 | 1.1510 | 0.5040 | 1.0640 | 0.5220 |
| A_51_P218924 | RIKEN cDNA 1110008E08 gene 1110008E08Rik | 441 | 1.3970 | 0.8260 | 0.9630 | 1.0620 | 0.2981 |
| | AK003565 | | | | | | |
| A_52_P229536 | CD44 antigen Cd44 AK045226 | 442 | 1.1420 | 1.0790 | 0.9590 | 1.0600 | 0.0930 |
| A_51_P246924 | RIKEN cDNA 2700055K07 gene 2700055K07Rik | 443 | 1.3170 | 0.9680 | 0.8780 | 1.0543 | 0.2319 |
| | NM_026481 | | | | | | |
| A_51_P447874 | Heat shock protein family, member 7 | 419 | 1.1070 | 1.0110 | 1.0390 | 1.0523 | 0.0494 |
| | (cardiovascular) Hspb7 NM_013868 | | | | | | |
| A_51_P237893 | Integrin beta 3 Itgb3 NM_016780 | 444 | 1.2210 | 0.9250 | 1.0040 | 1.0500 | 0.1533 |
| A_52_P533707 | Cholinergic receptor, nicotinic, alpha polypeptide 1 | 445 | 1.0870 | 1.0570 | 0.9750 | 1.0397 | 0.0580 |
| | (muscle) Chrna1 NM_007389 | | | | | | |
| A_51_P170059 | Small proline rich-like 10 Sprrl10 NM_025420 | 446 | 1.2860 | 0.8720 | 0.9600 | 1.0393 | 0.2181 |
| A_51_P346445 | Heat shock protein family, member 7 | 419 | 1.3270 | 0.8950 | 0.8780 | 1.0333 | 0.2545 |
| | (cardiovascular) Hspb7 NM_013868 | | | | | | |
| A_51_P364788 | Myosin, heavy polypeptide 1, skeletal muscle, | 447 | 1.5260 | 1.1020 | 0.4480 | 1.0253 | 0.5431 |
| | adult Myh1 XM_354615 | | | | | | |
| A_51_P283473 | RIKEN cDNA 1110018M03 gene 1110018M03Rik | 448 | 1.0570 | 0.7470 | 1.2610 | 1.0217 | 0.2588 |
| | NM_026271 | | | | | | |
| A_51_P347965 | Agouti related protein Agrp NM_007427 | 449 | 1.1760 | 0.7760 | 1.0700 | 1.0073 | 0.2072 |
| A_51_P236287 | Scaffold attachment factor B Safb AK087504 | 450 | 1.4750 | 1.0150 | 0.5240 | 1.0047 | 0.4756 |
| A_51_P462271 | Aggrecan 1 Agc1 NM_007424 | 451 | 0.8220 | 1.0590 | 1.1300 | 1.0037 | 0.1613 |
| A_52_P105537 | Nephroblastoma overexpressed gene Nov | 452 | 1.2660 | 0.9040 | 0.8350 | 1.0017 | 0.2315 |
| | NM_010930 | | | | | | |
| A_51_P356942 | Tripartite motif-containing 55 Trim55 XM_355438 | 453 | 1.2100 | 0.7340 | 1.0580 | 1.0007 | 0.2431 |
| A_52_P111390 | 3-phosphoinositide dependent protein kinase-1 | 454 | 1.1550 | 0.8000 | 1.0450 | 1.0000 | 0.1817 |
| | Pdpk1 NM_011062 | | | | | | |

B. Genes Downregulated at Least Two Fold in P2Y1/P2Y2 Double Knockout Mice

| | | | | | | | |
|---|---|---|---|---|---|---|---|
| A_52_P249544 | DNA segment, Chr 5, Brigham & Women's | 455 | −1.9510 | −0.1980 | −0.8850 | −1.0113 | 0.8833 |
| | Genetics 0860 expressed D5Bwg0860e | | | | | | |
| | NM_027530 | | | | | | |
| A_52_P620290 | Establishment of cohesion 1 homolog 1 (S. cerevisiae) | 456 | −1.0070 | −0.8320 | −1.2030 | −1.0140 | 0.1856 |
| | Esco1 BC008220 | | | | | | |
| A_51_P338485 | Aldehyde dehydrogenase family 6, subfamily A1 | 457 | −0.9380 | −1.0760 | −1.0550 | −1.0230 | 0.0744 |
| | Aldh6a1 NM_134042 | | | | | | |
| A_52_P138126 | 6-phosphofructo-2-kinase/fructose-2,6- | 458 | −1.2350 | −0.6930 | −1.1580 | −1.0287 | 0.2932 |
| | biphosphatase 3 Pfkfb3 NM_133232 | | | | | | |
| A_52_P241742 | RIKEN cDNA 2010003O02 gene 2010003O02Rik | 459 | −1.4710 | −0.9070 | −0.7100 | −1.0293 | 0.3950 |
| | AK008077 | | | | | | |
| A_52_P490863 | Nucleolar protein family A, member 3 Nola3 | 460 | −1.2500 | −0.9120 | −0.9450 | −1.0357 | 0.1864 |
| | NM_025403 | | | | | | |
| A_52_P536947 | Cytoplasmic FMR1 interacting protein 2 Cyfip2 | 461 | −0.8480 | −1.0820 | −1.1840 | −1.0380 | 0.1723 |
| | NM_133769 | | | | | | |
| A_51_P282268 | Small nuclear RNA activating complex, | 462 | −0.9210 | −1.2020 | −1.0650 | −1.0627 | 0.1405 |
| | polypeptide 1 Snapc1 NM_178392 | | | | | | |
| A_52_P115787 | Growth factor receptor bound protein 10 Grb10 | 463 | −1.0920 | −0.7340 | −1.3650 | −1.0637 | 0.3165 |
| | NM_010345 | | | | | | |
| A_52_P402761 | Establishment of cohesion 1 homolog 1 (S. cerevisiae) | 464 | −1.0600 | −0.7620 | −1.3770 | −1.0663 | 0.3075 |
| | Esco1 XM_484702 | | | | | | |
| A_52_P254817 | Resistin like alpha Retnla NM_020509 | 465 | −1.0950 | −1.1330 | −0.9850 | −1.0710 | 0.0769 |
| A_51_P512072 | Aminolevulinate, delta-, dehydratase Alad | 466 | −0.8350 | −1.1580 | −1.2260 | −1.0730 | 0.2089 |
| | NM_008525 | | | | | | |
| A_51_P189334 | TIGR Accession No. TC1414310 | 467 | −1.2830 | −0.8660 | −1.0920 | −1.0803 | 0.2087 |
| A_51_P511015 | Frizzled homolog 9 (Drosophila) Fzd9 AK021164 | 468 | −1.2030 | −1.2030 | −0.8970 | −1.1010 | 0.1767 |
| A_52_P168097 | ATPase, Ca++ transporting, cardiac muscle, slow | 469 | −0.7390 | −0.9170 | −1.6640 | −1.1067 | 0.4908 |
| | twitch 2 Atp2a2 NM_009722 | | | | | | |
| A_52_P81038 | Similar to RIKEN cDNA 4832428D23 gene | 470 | −0.9450 | −1.2110 | −1.1770 | −1.1110 | 0.1448 |
| | LOC433294 AK041301 | | | | | | |
| A_52_P1101647 | Ankyrin repeat and SOCS box-containing protein | 471 | −0.9440 | −1.2250 | −1.1820 | −1.1170 | 0.1514 |
| | 15 Asb15 AK079418 | | | | | | |
| A_52_P40777 | Rho GTPase activating protein 12 Arhgap12 | 472 | −0.6940 | −1.1030 | −1.5970 | −1.1313 | 0.4522 |
| | AK037784 | | | | | | |
| A_51_P408989 | RIKEN cDNA 2810055F11 gene 2810055F11Rik | 473 | −1.1770 | −1.1250 | −1.2020 | −1.1680 | 0.0393 |
| | NM_026038 | | | | | | |
| A_51_P357606 | Phytanoyl-CoA dioxygenase domain containing 1 | 474 | −1.3340 | −1.2820 | −0.9310 | −1.1823 | 0.2192 |
| | Phyhd1 NM_172267 | | | | | | |
| A_51_P389265 | Adiponutrin Adpn NM_054088 | 475 | −1.2810 | −1.2770 | −1.0670 | −1.2083 | 0.1224 |
| A_51_P439426 | acetyl-Coenzyme A carboxylase alpha Acac | 476 | −1.7690 | −1.0400 | −0.9340 | −1.2477 | 0.4546 |
| | NM_133360 | | | | | | |
| A_51_P140237 | Four and a half LIM domains 2 Fhl2 NM_010212 | 477 | −1.5080 | −1.5360 | −0.8530 | −1.2990 | 0.3865 |
| A_51_P212491 | 6-phosphofructo-2-kinase/fructose-2,6- | 458 | −1.3170 | −1.3830 | −1.2800 | −1.3267 | 0.0522 |
| | biphosphatase 3 Pfkfb3 NM_133232 | | | | | | |
| A_52_P395228 | Nicotinamide nucleotide transhydrogenase Nnt | 478 | −1.4990 | −1.2990 | −1.2180 | −1.3387 | 0.1446 |
| | NM_008710 | | | | | | |
| A_51_P451075 | ATPase, Ca++ transporting, cardiac muscle, slow | 469 | −1.3930 | −1.3400 | −1.3490 | −1.3607 | 0.0284 |
| | twitch 2 Atp2a2 NM_009722 | | | | | | |
| A_51_P387239 | Interferon inducible GTPase 1 ligp1 NM_021792 | 479 | −1.5740 | −1.6010 | −1.0210 | −1.3987 | 0.3273 |
| A_51_P465582 | Haloacid dehalogenase-like hydrolase domain | 480 | −1.8530 | −1.2230 | −1.1830 | −1.4197 | 0.3758 |
| | containing 3 Hdhd3 NM_024257 | | | | | | |
| A_51_P470715 | Cytokine inducible SH2-containing protein Cish | 481 | −1.2390 | −1.7290 | −1.3000 | −1.4227 | 0.2670 |
| | NM_009895 | | | | | | |
| A_52_P171166 | cDNA sequence BC048679 BC048679 | 482 | −1.6960 | −1.3070 | −1.3690 | −1.4573 | 0.2090 |
| | NM_183143 | | | | | | |
| A_51_P123879 | Steroid 5 alpha-reductase 2-like 2 Srd5a2l2 | 483 | −1.8060 | −1.3600 | −1.8480 | −1.6713 | 0.2704 |
| | NM_153801 | | | | | | |
| A_51_P110830 | A disintegrin-like and metalloprotease (reprolysin | 216 | −1.9670 | −1.4160 | −1.7400 | −1.7077 | 0.2769 |
| | type) with thrombospondin type 1 motif, 8 Adamts8 | | | | | | |
| | NM_013906 | | | | | | |
| A_52_P413395 | Sarcolipin Sln NM_025540 | 161 | −1.8150 | −1.7600 | −1.5900 | −1.7217 | 0.1173 |
| A_52_P100252 | Fatty acid synthase Fasn AK080374 | 484 | −1.1940 | −1.8030 | −2.2670 | −1.7547 | 0.5381 |
| A_51_P161308 | Solute carrier family 22 (organic cation | 485 | −1.8190 | −2.0430 | −1.6100 | −1.8240 | 0.2165 |
| | transporter), member 2 Slc22a2 NM_013667 | | | | | | |
| A_51_P408729 | 3-phosphoglycerate dehydrogenase Phgdh | 486 | −1.7310 | −1.9680 | −1.9010 | −1.8667 | 0.1222 |
| | NM_016966 | | | | | | |
| A_52_P509020 | A disintegrin-like and metalloprotease (reprolysin | 216 | −2.3950 | −1.5440 | −1.7300 | −1.8897 | 0.4474 |
| | type) with thrombospondin type 1 motif, 8 Adamts8 | | | | | | |
| | NM_013906 | | | | | | |
| A_51_P321126 | Fatty acid synthase Fasn AK080374 | 484 | −1.1730 | −2.1560 | −2.4010 | −1.9100 | 0.6499 |
| A_52_P547662 | Purinergic receptor P2Y, G-protein coupled 1 | 487 | −3.9400 | −3.5340 | −3.9930 | −3.8223 | 0.2511 |
| | P2ry1 NM_008772 | | | | | | |
| A_51_P239673 | Hypoxanthine guanine phosphoribosyl transferase | 488 | −4.2080 | −4.6210 | −4.8350 | −4.5547 | 0.3187 |
| | 1 Hprt1 NM_013556 | | | | | | |

^{a,b}The data is presented in the same fashion as described hereinabove with respect to Tables 1 and 2.

### Example 4

### Gene Expression Analysis of Human Tenocytes with and without Exposure to Interleukin 1β

Human tendon epitenon cells from the flexor digitorum profundus (FDP) were collected from surgical specimens and were maintained in Medium 199 (GIBCO®, Invitrogen Corp., Carlsbad, Calif., United States of America) containing 10% fetal bovine serum (FBS; HyClone, Logan, Utah, United States of America), 20 mM Hepes (pH 7.2; GIBCO®), 1% penicillin/streptomycin solution (GIBCO®). Cells were allowed to attach and spread for 24 hours before addition of 100 pM recombinant human IL-1β (rhIL-1β). The serum concentration was reduced from 10% to 2% upon addition of rhIL-1β. Culture medium was changed daily. Cells at passage 3 were treated with 100 pM IL-1β for 6 hours, and untreated cells after an equivalent time in culture were used as controls.

For the human tenocytes treated with or without IL-1β, about 3600 genes out of 20 k were changed at least about 2 fold, 1000 genes were changed at least about 4 fold, 275 genes were changed at least about 8 fold, 80 genes were changed at least about 16 fold, 22 genes were changed at least about 32 fold, and 3 genes were changed at least about 64 fold. Expression level differences of some of the MMPs were among the most dramatic changes observed. However, the alteration of mucin gene expression by IL-1β was one of several unexpected findings.

TABLE 4
Comparison of Gene Expression Levels Between Human Tenocytes Exposed In Vitro to
Human Recombinant IL-1β Versus Unexposed Human Tenocytes^{a}

| | | | |
|---|---|---|---|
| | | SEQ | +1L-1β |
| | | ID | vs. |
| CLID | NAME^{b} | NO. | Control |

A. Genes Upregulated at Least Eight Fold by hIL-1β Treatment

| | | | |
|---|---|---|---|
| CGEN_HUM_1006382_1 | CXCL2 Chemokine (C—X—C motif) ligand 2 NM_002089 | 489 | 6.3150 |
| CGEN_HUM_1009916_1 | CXCL3 Chemokine (C—X—C motif) ligand 3 NM_002090 | 490 | 5.5610 |
| CGEN_HUM_1011980_1 | G0S2 Putative lymphocyte G0/G1 switch gene NM_015714 | 491 | 5.5430 |
| CGEN_HUM_1012345_1 | COL1A2 Collagen, type I, alpha 2 L47668 | 492 | 5.2770 |
| CPEROU_OLIGO_32_0 | IL1A Interleukin 1, alpha NM_000575 | 493 | 5.1970 |
| CGEN_HUM_1006519_1 | IL8 Interleukin 8 M17017 | 494 | 5.1050 |
| CGEN_HUM_1007899_1 | TNFAIP2 Tumor necrosis factor, alpha-induced protein 2 NM_006291 | 495 | 5.0750 |
| CGEN_HUM_1008675_1 | COL6A2 Type VI collagen alpha 2 chain precursor M20777; AY029208 | 496 | 5.0680 |
| CGEN_HUM_1006376_1 | PTX3 Pentaxin-related gene, rapidly induced by IL-1 beta NM_002852 | 497 | 5.0230 |
| CGEN_HUM_1000105_1 | IER3 Immediate early response 3 NM_003897 | 498 | 5.0070 |
| CPEROU_OLIGO_633_0 | ADAM15 A disintegrin and metalloproteinase domain 15 (metargidin) AA292676 | 499 | 4.9940 |
| CGEN_HUM_1006393_1 | CSF3 Colony stimulating factor 3 (granulocyte) NM_000759 | 500 | 4.9790 |
| CPEROU_OLIGO_884_0 | MSN Moesin R22977 | 501 | 4.8100 |
| CPEROU_OLIGO_782_0 | FOXC1 Forkhead box C1 N22552 | 502 | 4.8000 |
| CGEN_HUM_1006585_1 | SERPINB2 Serine (or cysteine) proteinase inhibitor, clade B (ovalbumin), member 2 | 503 | 4.7560 |
| | NM_002575 | | |
| CGEN_HUM_1011802_1 | MMP2 Matrix metalloproteinase 2 (gelatinase A, 72 kDa gelatinase, 72 kDa type IV | 504 | 4.7410 |
| | collagenase) X58968 | | |
| CGEN_HUM_1006064_1 | CCL20 Chemokine (C-C motif) ligand 20 NM_004591 | 505 | 4.6790 |
| CPEROU_OLIGO_80_0 | BF B-factor, properdin AA401441 | 506 | 4.6590 |
| CGEN_HUM_1006022_1 | PTGS2 Prostaglandin-endoperoxide synthase 2 (prostaglandin G/H synthase and | 507 | 4.6390 |
| | cyclooxygenase) NM_000963 | | |
| CGEN_HUM_1011318_1 | MAGED2 Melanoma antigen, family D, 2 U92544 | 508 | 4.5690 |
| CGEN_HUM_1018359_1 | Full length insert cDNA clone ZC30C07 AF086184 | 509 | 4.4930 |
| CGEN_HUM_1006490_1 | G1P3 Interferon, alpha-inducible protein (clone IFI-6-16) NM_002038 | 510 | 4.4900 |
| CGEN_HUM_1011497_1 | EFEMP2 EGF-containing fibulin-like extracellular matrix protein 2 NM_016938 | 511 | 4.4640 |
| CGEN_HUM_1010898_1 | SLC39A14 Solute carrier family 39 (zinc transporter), member 14 D31887 | 512 | 4.4080 |
| CGEN_HUM_1007394_1 | MLF2 Myeloid leukemia factor 2 NM_005439 | 513 | 4.4040 |
| CGEN_HUM_1007970_1 | FGF2 Fibroblast growth factor 2 (basic) NM_002006 | 514 | 4.3950 |
| CPEROU_OLIGO_447_0 | HepG2 3′ region Mbol cDNA, clone hmd2a08m3. AA487750 | 515 | 4.3620 |
| CGEN_HUM_1006508_1 | SDF1B cytokine SDF-1-beta U16752 | 516 | 4.3570 |
| CGEN_HUM_1010229_1 | CXCL6 Chemokine (C—X—C motif) ligand 6 (granulocyte chemotactic protein 2) | 517 | 4.2770 |
| | Y08770 | | |
| CGEN_HUM_1007360_1 | LAMP1 Lysosomal-associated membrane protein 1 NM_005561 | 518 | 4.1880 |
| CPEROU_OLIGO_41_0 | MT1X Metallothionein 1X N80129 | 519 | 4.1520 |
| CPEROU_OLIGO_283_0 | HMGA1 High mobility group AT-hook 1 AA448261 | 520 | 4.1080 |
| CGEN_HUM_1006430_1 | IL1A interleukin 1 alpha M28983 | 521 | 4.0760 |
| CGEN_HUM_1009830_1 | TGFBR2 Transforming growth factor, beta receptor II (70/80 kDa) NM_003242 | 522 | 4.0480 |
| CGEN_HUM_1008002_1 | ATF5 Activating transcription factor 5 NM_012068 | 523 | 4.0390 |
| CGEN_HUM_1002530_1 | HNRPUL1 Heterogeneous nuclear ribonucleoprotein U-like 1 NM_007040 | 524 | 4.0350 |
| CGEN_HUM_1008744_1 | CLSTN1 Calsyntenin 1 NM_014944 | 525 | 4.0210 |
| CGEN_HUM_1008447_1 | APP Amyloid beta (A4) precursor protein (protease nexin-II, Alzheimer disease) | 526 | 4.0120 |
| | M35675 | | |
| CGEN_HUM_1017516_1 | INHBA Inhibin, beta A (activin A, activin AB alpha polypeptide) AK001903 | 527 | 4.0040 |
| CGEN_HUM_1009861_1 | GNB2 Guanine nucleotide binding protein (G protein), beta polypeptide 2 | 528 | 3.9610 |
| | NM_005273 | | |
| CGEN_HUM_1008791_1 | PCQAP PC2 (positive cofactor 2, multiprotein complex) glutamine/Q-rich-associated | 529 | 3.9450 |
| | protein NM_015889 | | |
| CGEN_HUM_1002984_1 | EIF4G1 Eukaryotic translation initiation factor 4 gamma, 1 NM_004953 | 530 | 3.9250 |
| CGEN_HUM_1010841_1 | ELN Elastin (supravalvular aortic stenosis, Williams-Beuren syndrome) NM_000501 | 531 | 3.8910 |
| CGEN_HUM_1003129_1 | EHD1 EH-domain containing 1 NM_006795 | 532 | 3.8880 |
| CGEN_HUM_1010565_1 | ADRM1 Adhesion regulating molecule 1 NM_007002 | 533 | 3.8830 |
| CPEROU_OLIGO_609_0 | GENBANK ® Accession No. H69582 | 534 | 3.8710 |
| CGEN_HUM_1008656_1 | COL6A2 type VI collagen alpha 2 chain precursor X15881; AY029208 | 535 | 3.8440 |
| CGEN_HUM_1000304_1 | HAS1 Hyaluronan synthase 1 NM_001523 | 536 | 3.8120 |
| CGEN_HUM_1005956_1 | ARPC1B Actin related protein 2/3 complex, subunit 1B, 41 kDa NM_005720 | 537 | 3.8100 |
| CGEN_HUM_1008636_1 | LTBP3 Latent transforming growth factor beta binding protein 3 AF135960 | 538 | 3.7920 |
| CGEN_HUM_1007887_1 | SMOX Spermine oxidase NM_019025 | 539 | 3.7900 |
| CGEN_HUM_1010254_1 | LAMB2 Laminin, beta 2 (laminin S) NM_002292 | 540 | 3.7810 |
| CGEN_HUM_1009002_1 | TRIP10 Thyroid hormone receptor interactor 10 AJ000414 | 541 | 3.7630 |
| CGEN_HUM_1017411_1 | FBS1 Fibrosin 1 AK022551 | 542 | 3.7410 |
| CGEN_HUM_1006495_1 | CSF2 Colony stimulating factor 2 (granulocyte-macrophage) NM_000758 | 543 | 3.7400 |
| CGEN_HUM_1008484_1 | HUMC6A2A1 alpha-2 collagen type VI, alpha-2 collagen type VI-a, and alpha-2 | 544 | 3.7360 |
| | collagen type VI-a′ gene, exons 6, 5, 4, and 3 M34571 | | |
| CGEN_HUM_1007239_1 | TUBB2 Tubulin, beta 2 NM_001069 | 545 | 3.7130 |
| CPEROU_OLIGO_955_0 | CCL2 Chemokine (C-C motif) ligand 2 AA425102 | 546 | 3.7070 |
| CPEROU_OLIGO_627_0 | CYCS Cytochrome c, somatic NM_018947 | 547 | 3.6750 |
| CGEN_HUM_1002036_1 | JUNB Jun B proto-oncogene NM_002229 | 548 | 3.6750 |
| CGEN_HUM_1006668_1 | SOD2 Superoxide dismutase 2, mitochondrial M36693 | 549 | 3.6720 |
| CGEN_HUM_1007822_1 | MAPK3 Mitogen-activated protein kinase 3 X60188 | 550 | 3.6480 |
| CGEN_HUM_1013195_1 | HUMO40 osteonectin, 5′UTR region D28381 | 551 | 3.5740 |
| CGEN_HUM_1018077_1 | C9orf26 Chromosome 9 open reading frame 26 (NF-HEV) AB024518 | 552 | 3.5540 |
| CGEN_HUM_1008682_1 | HUMC6A2A2 alpha-2 collagen type VI and alpha-2 collagen type VI-a gene, exons | 553 | 3.5440 |
| | 2a and 2b M34572 | | |
| CGEN_HUM_1007336_1 | NBL1 Neuroblastoma, suppression of tumorigenicity 1 NM_005380 | 554 | 3.5250 |
| CPEROU_OLIGO_826_0 | ITM2C Integral membrane protein 2C AA034213 | 555 | 3.5210 |
| CGEN_HUM_1005878_1 | SPATS2 Spermatogenesis associated, serine-rich 2 AK023202 | 556 | 3.5200 |
| CGEN_HUM_1005594_1 | MT2A Metallothionein 2A NM_005953 | 557 | 3.5060 |
| CGEN_HUM_1008786_1 | PRO1855 Hypothetical protein PRO1855 NM_018509 | 558 | 3.5000 |
| CGEN_HUM_1003165_1 | MAP2K2 Mitogen-activated protein kinase kinase 2 L11285 | 559 | 3.4990 |
| CGEN_HUM_1003829_1 | MMP1 Matrix metalloproteinase 1 (interstitial collagenase) NM_002421 | 560 | 3.4800 |
| CGEN_HUM_1012020_1 | ADDA alpha-adducin mRNA, partial sequence, alternatively spliced S70313 | 561 | 3.4680 |
| CGEN_HUM_1008726_1 | CD44 CD44 antigen (homing function and Indian blood group system) M59040 | 562 | 3.4590 |
| CGEN_HUM_1007048_1 | CNOT3 CCR4-NOT transcription complex, subunit 3 NM_014516 | 563 | 3.4550 |
| CPEROU_OLIGO_258_0 | FLOT2 Flotillin 2 R72913 | 564 | 3.4500 |
| CGEN_HUM_1012392_1 | C6orf106 Chromosome 6 open reading frame 106 AF052106 | 565 | 3.4450 |
| CGEN_HUM_1015858_1 | cDNA FLJ13836 fis, clone THYRO1000734 AK023898 | 566 | 3.4350 |
| CGEN_HUM_1009055_1 | LOC440460 X99662 | 567 | 3.4180 |
| CGEN_HUM_1006686_1 | HSPB7 Heat shock 27 kDa protein family, member 7 (cardiovascular) NM_014424 | 568 | 3.4120 |
| CPEROU_OLIGO_952_0 | STAT1 Signal transducer and activator of transcription 1, 91 kDa AA486367 | 569 | 3.4120 |
| CGEN_HUM_1006431_1 | IL1B Interleukin 1, beta M15330 | 570 | 3.4000 |
| CGEN_HUM_1017927_1 | LOC162427 Hypothetical protein LOC162427 L38937 | 571 | 3.3910 |
| CGEN_HUM_1006458_1 | IL1RN Interleukin 1 receptor antagonist M55646 | 572 | 3.3430 |
| CGEN_HUM_1003212_1 | LOXL2 Lysyl oxidase-like 2 NM_002318 | 573 | 3.3420 |
| CGEN_HUM_1011355_1 | NOL6 Nucleolar protein family 6 (RNA-associated) AK025612 | 574 | 3.3390 |
| CGEN_HUM_1011264_1 | TSPYL2 TSPY-like 2 AF273046 | 575 | 3.3380 |
| CGEN_HUM_1009728_1 | IFNAR2 Interferon (alpha, beta and omega) receptor 2 NM_000874 | 576 | 3.3370 |
| CPEROU_OLIGO_698_0 | CORO1C Coronin, actin binding protein, 1C AA126947 | 577 | 3.3330 |
| CGEN_HUM_1006712_1 | MT1L Metallothionein 1L X97261 | 578 | 3.3330 |
| CGEN_HUM_1005168_1 | ABCC1 ATP-binding cassette, sub-family C (CFTR/MRP), member 1 NM_004996 | 579 | 3.3230 |
| CGEN_HUM_1002999_1 | WBSCR1 Williams-Beuren syndrome chromosome region 1 D26068 | 580 | 3.3220 |
| CGEN_HUM_1016751_1 | cDNA DKFZp564E233 (from clone DKFZp564E233) AL049260 | 581 | 3.3120 |
| CGEN_HUM_1017629_1 | FLJ20701 Hypothetical protein FLJ20701 NM_017933 | 582 | 3.3120 |
| CGEN_HUM_1010466_1 | AK026383 FLJ22730 fis, clone HSI15793, highly similar to AF004162 Homo sapiens | 583 | 3.3100 |
| | nickel-specific induction protein (Cap43) AK026383 | | |
| CGEN_HUM_1003426_1 | PPP2R1A Protein phosphatase 2 (formerly 2A), regulatory subunit A (PR 65), alpha | 584 | 3.3040 |
| | isoform NM_014225 | | |
| CGEN_HUM_1009464_1 | PPAP2C Phosphatidic acid phosphatase type 2C NM_003712 | 585 | 3.2970 |
| CPEROU_OLIGO_527_0 | MRPS22 Mitochondrial ribosomal protein S22 N62924 | 586 | 3.2860 |
| CGEN_HUM_1009759_1 | BSG Basigin (OK blood group) NM_001728 | 587 | 3.2670 |
| CGEN_HUM_1007904_1 | PCTK1 PCTAIRE protein kinase 1 NM_006201 | 588 | 3.2480 |
| CPEROU_OLIGO_397_0 | PFKP Phosphofructokinase, platelet AA608558 | 589 | 3.2430 |
| CGEN_HUM_1012268_1 | HIC1 Hypermethylated in cancer 1 NM_006497 | 590 | 3.2200 |
| CPEROU_OLIGO_608_0 | GENBANK ® Accession No. H69582 | 591 | 3.2120 |
| CPEROU_OLIGO_272_0 | GPX3 Glutathione peroxidase 3 (plasma) AA664180 | 592 | 3.2090 |
| CGEN_HUM_1007390_1 | AKT1 V-akt murine thymoma viral oncogene homolog 1 NM_005163 | 593 | 3.2060 |
| CGEN_HUM_1002450_1 | PABPC1 Poly(A) binding protein, cytoplasmic 1 NM_002568 | 594 | 3.2010 |
| CGEN_HUM_1006699_1 | TRA1 Tumor rejection antigen (gp96) 1 NM_003299 | 595 | 3.1970 |
| CGEN_HUM_1008785_1 | ADAMTS7 A disintegrin-like and metalloprotease (reprolysin type) with | 596 | 3.1950 |
| | thrombospondin type 1 motif, 7 AL110226 | | |
| CGEN_HUM_1005849_1 | LOC51238 hypothetical protein LOC51238 NM_016465 | 597 | 3.1900 |
| CPEROU_OLIGO_950_0 | SRPK1 SFRS protein kinase 1 NM_003137 | 598 | 3.1880 |
| CGEN_HUM_1007529_1 | MLLT1 Myeloid/lymphoid or mixed-lineage leukemia (trithorax homolog, Drosophila); | 599 | 3.1870 |
| | translocated to, 1 NM_005934 | | |
| CPEROU_OLIGO_937_0 | RBL2 Retinoblastoma-like 2 (p130) NM_005611 | 600 | 3.1820 |
| CPEROU_OLIGO_454_0 | MGC39900 Hypothetical protein MGC39900 N91887 | 601 | 3.1710 |
| CGEN_HUM_1002039_1 | CEBPB CCAAT/enhancer binding protein (C/EBP), beta NM_005194 | 602 | 3.1660 |
| CGEN_HUM_1006872_1 | SYMPK Symplekin NM_004819 | 603 | 3.1580 |
| CGEN_HUM_1007208_1 | KIF22 Kinesin family member 22 NM_007317 | 604 | 3.1540 |
| CGEN_HUM_1007565_1 | IL15RA Interleukin 15 receptor, alpha NM_002189 | 605 | 3.1450 |
| CGEN_HUM_1010131_1 | STAT1 Signal transducer and activator of transcription 1, 91 kDa NM_007315 | 606 | 3.1420 |
| CGEN_HUM_1009895_1 | GPR Putative G protein coupled receptor NM_007223 | 607 | 3.1310 |
| CGEN_HUM_1007634_1 | PHB Prohibitin NM_002634 | 608 | 3.1240 |
| CGEN_HUM_1018847_1 | VDP Vesicle docking protein p115 NM_003715 | 609 | 3.1160 |
| CGEN_HUM_1002994_1 | EIF5A Eukaryotic translation initiation factor 5A NM_001970 | 610 | 3.1100 |
| CGEN_HUM_1004900_1 | DKFZP564B167 DKFZP564B167 protein NM_015415 | 611 | 3.1060 |
| CPEROU_OLIGO_862_0 | SLC39A6 Solute carrier family 39 (zinc transporter), member 6 H29315 | 612 | 3.0650 |
| CGEN_HUM_1007546_1 | NAB2 NGFI-A binding protein 2 (EGR1 binding protein 2) NM_005967 | 613 | 3.0640 |
| CPEROU_OLIGO_532_0 | C1R Complement component 1, r subcomponent T69603 | 614 | 3.0510 |
| CGEN_HUM_1006456_1 | MCP-3 monocyte chemotactic protein-3 X72308 | 615 | 3.0500 |
| CGEN_HUM_1003896_1 | C1R Complement component 1, r subcomponent NM_001733 | 616 | 3.0400 |
| CGEN_HUM_1011935_1 | MT-1g metallothionein MT-1g isoform S68954 | 617 | 3.0390 |
| CPEROU_OLIGO_71_0 | GENBANK ® Accession No. H66070 | 618 | 3.0330 |
| CGEN_HUM_1008673_1 | TPBG Trophoblast glycoprotein NM_006670 | 619 | 3.0300 |
| CGEN_HUM_1010055_1 | PTK2 PTK2 protein tyrosine kinase 2 NM_005607 | 620 | 3.0170 |
| CGEN_HUM_1004641_1 | PIK4CB Phosphatidylinositol 4-kinase, catalytic, beta polypeptide NM_002651 | 621 | 3.0140 |
| CGEN_HUM_1008056_1 | PPP1CC Protein phosphatase 1, catalytic subunit, gamma isoform NM_002710 | 622 | 3.0140 |
| CPEROU_OLIGO_276_0 | CXCL1 Chemokine (C—X—C motif) ligand 1 (melanoma growth stimulating activity, | 623 | 3.0110 |
| | alpha) W42723 | | |

B. Genes Downregulated at Least Eight Fold by Treatment with hIL-1β Treatment

| | | | |
|---|---|---|---|
| CGEN_HUM_1013169_1 | FLJ12800 Hypothetical protein FLJ12800 AK023691 | 624 | −3.0000 |
| CGEN_HUM_1018287_1 | NAPE-PLD N-acyl-phosphatidylethanolamine-hydrolyzing phospholipase D | 625 | −3.0050 |
| | AK000801 | | |
| CGEN_HUM_1006696_1 | SERPINH1 Serine (or cysteine) proteinase inhibitor, clade H (heat shock protein 47), | 626 | −3.0620 |
| | member 1, (collagen binding protein 1) NM_001235 | | |
| CGEN_HUM_1017475_1 | MGC3200 Hypothetical protein LOC284615 AL359622 | 627 | −3.0690 |
| CGEN_HUM_1018837_1 | RAPH1 Ras association (RaIGDS/AF-6) and pleckstrin homology domains 1 | 628 | −3.0700 |
| | AF086189 | | |
| CPEROU_OLIGO_197_0 | Transcribed locus AA056377 | 629 | −3.0770 |
| CGEN_HUM_1000972_1 | COH1 Cohen syndrome 1 NM_017890 | 630 | −3.0870 |
| CPEROU_OLIGO_155_0 | Full-length cDNA clone CS0CAP002YO01 of Thymus of Homo sapiens (human) | 631 | −3.0880 |
| | W87826 | | |
| CGEN_HUM_1009060_1 | RASAL2 RAS protein activator like 2 NM_004841 | 632 | −3.1120 |
| CGEN_HUM_1002744_1 | CTBP2 C-terminal binding protein 2 NM_001329 | 633 | −3.1140 |
| CGEN_HUM_1005622_1 | ARF3 ADP-ribosylation factor 3 NM_001659 | 634 | −3.1200 |
| CGEN_HUM_1006998_1 | MNS1 Meiosis-specific nuclear structural protein 1 NM_018365 | 635 | −3.1210 |
| CGEN_HUM_1007685_1 | FLJ10385 Hypothetical protein FLJ10385 U58658 | 636 | −3.1370 |
| CGEN_HUM_1007662_1 | HRASLS HRAS-like suppressor NM_020386 | 637 | −3.1410 |
| CGEN_HUM_1006557_1 | PAX5 Paired box gene 5 (B-cell lineage specific activator) NM_016734 | 638 | −3.1800 |
| CGEN_HUM_1016508_1 | cDNA: FLJ22769 fis, clone KAIA1316 AK026422 | 639 | −3.1840 |
| CGEN_HUM_1016177_1 | PTGDR Prostaglandin D2 receptor (DP) AK026202 | 640 | −3.1870 |
| CGEN_HUM_1015660_1 | cDNA FLJ11479 fis, clone HEMBA1001784 AK021541 | 641 | −3.1920 |
| CGEN_HUM_1001684_1 | ZNF205 Zinc finger protein 205 NM_003456 | 642 | −3.2060 |
| CGEN_HUM_1014309_1 | FLJ11151 Hypothetical protein FLJ11151 NM_018340 | 643 | −3.2080 |
| CGEN_HUM_1008916_1 | CNOT7 CCR4-NOT transcription complex, subunit 7 NM_013354 | 644 | −3.2130 |
| CGEN_HUM_1014020_1 | cDNA FLJ13605 fis, clone PLACE1010562 AK023667 | 645 | −3.2190 |
| CGEN_HUM_1003408_1 | PTPRN Protein tyrosine phosphatase, receptor type, N NM_002846 | 646 | −3.2230 |
| CGEN_HUM_1007205_1 | TUBD1 Tubulin, delta 1 NM_016261 | 647 | −3.2280 |
| CGEN_HUM_1012948_1 | CTNND2 Catenin (cadherin-associated protein), delta 2 (neural plakophilin-related | 648 | −3.2310 |
| | arm-repeat protein) AF056423 | | |
| CGEN_HUM_1017096_1 | G6PD Glucose-6-phosphate dehydrogenase M19866 | 649 | −3.2370 |
| CGEN_HUM_1018146_1 | LOC90110 Hypothetical protein LOC90110 AL117623 | 650 | −3.2720 |
| CGEN_HUM_1014942_1 | EIF5B Eukaryotic translation initiation factor 5B AK025799 | 651 | −3.2810 |
| CGEN_HUM_1013908_1 | GENBANK ® Accession No. U61100 | 652 | −3.2880 |
| CGEN_HUM_1005267_1 | SLC30A4 Solute carrier family 30 (zinc transporter), member 4 NM_013309 | 653 | −3.2940 |
| CGEN_HUM_1006072_1 | FPRL1 Formyl peptide receptor-like 1 NM_001462 | 654 | −3.3020 |
| CGEN_HUM_1013068_1 | LOC201158 Similar to CGI-148 protein AK022250 | 655 | −3.3160 |
| CGEN_HUM_1013140_1 | OR2F1 olfactory receptor, family 2, subfamily F, member 1 NM_012369 | 656 | −3.3320 |
| CGEN_HUM_1011543_1 | MYO9A Myosin IXA NM_006901 | 657 | −3.3530 |
| CGEN_HUM_1017078_1 | HUMCFMS01 transmembrane glycoprotein (c-fms) gene, exon 1, and platelet- | 658 | −3.3580 |
| | derived growth factor receptor (PDGF) gene, 3′UTR M25785 | | |
| CGEN_HUM_1014604_1 | MLSTD2 Male sterility domain containing 2 AK024967 | 659 | −3.3860 |
| CGEN_HUM_1015769_1 | cDNA FLJ12239 fis, clone MAMMA1001268 AK022301 | 660 | −3.3930 |
| CGEN_HUM_1005734_1 | RAB19B GTP-binding protein RAB19B AF091033 | 661 | −3.4080 |
| CGEN_HUM_1002596_1 | PRPF3 PRP3 pre-mRNA processing factor 3 homolog (yeast) NM_004698 | 662 | −3.4320 |
| CGEN_HUM_1014273_1 | P53AIP1 P53-regulated apoptosis-inducing protein 1 AB045832 | 663 | −3.4760 |
| CGEN_HUM_1016941_1 | QC2 QC2 geneX69081 | 664 | −3.4840 |
| CGEN_HUM_1017835_1 | TMEM35 Transmembrane protein 35 AK024146 | 665 | −3.4950 |
| CGEN_HUM_1017654_1 | GTCD1 glycosyltransferase-like domain containing 1, transcript variant 2 | 666 | −3.5010 |
| | NM_014118; NM_024659 | | |
| CGEN_HUM_1014548_1 | clone HEB4 Cri-du-chat region mRNA AF009287 | 667 | −3.5540 |
| CGEN_HUM_1006275_1 | MAP3K5 Mitogen-activated protein kinase kinase kinase 5 NM_005923 | 668 | −3.6390 |
| CGEN_HUM_1005939_1 | NUP155 Nucleoporin 155 kDa NM_004298 | 669 | −3.6390 |
| CGEN_HUM_1010762_1 | SCML2 Sex comb on midleg-like 2 (Drosophila) NM_006089 | 670 | −3.6500 |
| CGEN_HUM_1001704_1 | EVX1 Eve, even-skipped homeo box homolog 1 (Drosophila) NM_001989 | 671 | −3.6580 |
| CGEN_HUM_1009575_1 | EPHA6 EPH receptor A6 AL133666 | 672 | −3.6630 |
| CGEN_HUM_1003918_1 | MEP1B Meprin A, beta NM_005925 | 673 | −3.6670 |
| CGEN_HUM_1014069_1 | Clone IMAGE: 111510 mRNA sequence AF143870 | 674 | −3.6840 |
| CGEN_HUM_1012275_1 | Full length insert cDNA clone YB63G06 AF147362 | 675 | −3.6950 |
| CGEN_HUM_1004163_1 | MGC3123 Hypothetical protein MGC3123 AY007092 | 676 | −3.6980 |
| CGEN_HUM_1018482_1 | Full length insert cDNA clone ZD88D12 AF086474 | 677 | −3.7020 |
| CGEN_HUM_1017224_1 | C10orf18 Chromosome 10 open reading frame 18 AL049233 | 678 | −3.7480 |
| CGEN_HUM_1005136_1 | SAC Testicular soluble adenylyl cyclase NM_018417 | 679 | −3.7480 |
| CGEN_HUM_1013865_1 | cDNA clone IMAGE: 5278284, mRNA AK024371 | 680 | −3.7520 |
| CGEN_HUM_1009665_1 | KIAA1467 Serotonin-7 receptor pseudogene U86813 | 681 | −3.7890 |
| CGEN_HUM_1010847_1 | SALL2 Sal-like 2 (Drosophila) X98834 | 682 | −3.8340 |
| CGEN_HUM_1017708_1 | FMNL2 Formin-like 2 AL390143 | 683 | −3.8610 |
| CGEN_HUM_1005022_1 | ABCA12 ATP-binding cassette, sub-family A (ABC1), member 12 AL080207 | 684 | −3.8640 |
| CGEN_HUM_1010038_1 | NPY Neuropeptide Y NM_000905 | 685 | −3.9050 |
| CPEROU_OLIGO_37_0 | IL20 Interleukin 20 NM_018724 | 686 | −3.9280 |
| CGEN_HUM_1013103_1 | IL1RAPL1 Interleukin 1 receptor accessory protein-like 1 AL157478 | 687 | −3.9690 |
| CGEN_HUM_1012359_1 | LCHN LCHN protein AF116707 | 688 | −3.9700 |
| CGEN_HUM_1000029_1 | RHOH Ras homolog gene family, member H NM_004310 | 689 | −3.9710 |
| CGEN_HUM_1009159_1 | TNFRSF18 Tumor necrosis factor receptor superfamily, member 18 NM_004195 | 690 | −3.9960 |
| CGEN_HUM_1005100_1 | C16orf3 Chromosome 16 open reading frame 3 NM_001214 | 691 | −4.0860 |
| CGEN_HUM_1007404_1 | FGF5 Fibroblast growth factor 5 NM_004464 | 692 | −4.1240 |
| CGEN_HUM_1018152_1 | PLCXD2 Phosphatidylinositol-specific phospholipase C, X domain containing 2 | 693 | −4.1240 |
| | AF143877 | | |
| CGEN_HUM_1002550_1 | RBMY2FP RNA binding motif protein, Y-linked, family 2, member F pseudogene | 694 | −4.1690 |
| | U94387 | | |
| CGEN_HUM_1008501_1 | CHRDL2 Chordin-like 2 AL110168 | 695 | −4.1940 |
| CGEN_HUM_1014739_1 | HRMT1L1 HMT1 hnRNP methyltransferase-like 1 (S. cerevisiae) AL050065 | 696 | −4.1970 |
| CGEN_HUM_1007487_1 | NAG Neuroblastoma-amplified protein NM_015909 | 697 | −4.2130 |
| CGEN_HUM_1013840_1 | GENBANK ® Accession No. NM_018635 | 698 | −4.2170 |
| CGEN_HUM_1018020_1 | PARVB Parvin, beta AF147358 | 699 | −4.2230 |
| CGEN_HUM_1009154_1 | GNRH2 Gonadotropin-releasing hormone 2 NM_001501 | 700 | −4.2560 |
| CGEN_HUM_1012300_1 | SEL1L Sel-1 suppressor of lin-12-like (C. elegans) AK022015 | 701 | −4.2760 |
| CGEN_HUM_1008102_1 | KIF2C Kinesin family member 2C NM_006845 | 702 | −4.2960 |
| CGEN_HUM_1004037_1 | UBQLN3 Ubiquilin 3 NM_017481 | 703 | −4.3240 |
| CGEN_HUM_1010980_1 | GENBANK ® Accession No. NM_017973 | 704 | −4.3380 |
| CGEN_HUM_1016527_1 | cDNA clone YR22D05 AF085916 | 705 | −4.4100 |
| CGEN_HUM_1013092_1 | KLK12 Kallikrein 12 NM_019598 | 706 | −4.4890 |
| CGEN_HUM_1010428_1 | DHX34 DEAH box polypeptide 34 NM_014681 | 707 | −4.5320 |
| CGEN_HUM_1018667_1 | SYT9 Synaptotagmin IX AL137512 | 708 | −4.5470 |
| CGEN_HUM_1010012_1 | CCR6 Chemokine (C-C motif) receptor 6 NM_004367 | 709 | −4.6040 |
| CGEN_HUM_1018044_1 | EST from clone 76558, 5′ end AL110290 | 710 | −4.6380 |
| CGEN_HUM_1005434_1 | RYR1 Ryanodine receptor 1 (skeletal) J05200 | 711 | −4.7160 |
| CGEN_HUM_1016063_1 | RAB38 RAB38, member RAS oncogene family AK026725 | 712 | −4.7420 |
| CGEN_HUM_1014304_1 | SBF2 SET binding factor 2 U80769 | 713 | −4.7490 |
| CGEN_HUM_1010210_1 | SIRPB2 Signal-regulatory protein beta 2 NM_018556 | 714 | −4.7720 |
| CGEN_HUM_1005628_1 | RAB20 RAB20, member RAS oncogene family NM_017817 | 715 | −4.8360 |
| CGEN_HUM_1012516_1 | FLJ10786 Hypothetical protein FLJ10786 NM_018219 | 716 | −4.8700 |
| CGEN_HUM_1009809_1 | LOC55971 Insulin receptor tyrosine kinase substrate NM_018842 | 717 | −4.9950 |
| CGEN_HUM_1011590_1 | OR2L1P Olfactory receptor, family 2, subfamily L, member 1 pseudogene X64980 | 718 | −5.0820 |
| CGEN_HUM_1013476_1 | cDNA DKFZp434D1516 (from clone DKFZp434D1516) AL137284 | 719 | −5.1540 |
| CGEN_HUM_1013511_1 | GPR8 G protein-coupled receptor 8 NM_005286 | 720 | −5.3030 |
| CGEN_HUM_1012346_1 | PRO1048 hypothetical protein PRO1048 NM_018497 | 721 | −5.3920 |
| CGEN_HUM_1018788_1 | CALN1 Calneuron 1 AF070549 | 722 | −5.5170 |
| CGEN_HUM_1014948_1 | SGOL1 Shugoshin-like 1 (S. pombe) AK024292 | 723 | −6.2870 |
| CGEN_HUM_1014954_1 | cDNA DKFZp566P1546 (from clone DKFZp566P1546) AL050085 | 724 | −6.9370 |

^{a}The data presented in the column entitled “+1L-1β vs. Control” are presented in the form of a fold increase in IL-1β-treated cells versus control cells (i.e., no IL-1β treatment). The values are expressed as the log₂[fold increase] as before. In Table 4B, the means have negative values to indicate that these genes are downregulated by IL-1β treatment.
^{b}The descriptions that appear in the column headed by “NAME” include one or more of a gene name, a gene description, and one or more database accession numbers. All accession numbers are for the GENBANK ® database unless otherwise indicated.

### Example 5

### Gene Expression Analysis of Achilles Tendon Versus Other Tendons

Achilles tendon, flexor tendon, and tail tendon tissues were collected wild type mice and RNA was isolated and reverse transcribed as described above in General Materials and Methods. Mouse Achilles tendon (AT) RNAs were reverse transcribed into cDNAs labeled with Cyanine 3 (a green dye fluorophore; Cy3) while flexor tendon or tial tendon RNAs were labeled with cyanine 5 (a red dye fluorophore; Cy5). cDNAs from AT, flexor tendon, or tail tendon were pooled in equal proportions then hybridized with arrayed DNA sequences using the Agilent chip, with AT being compared to flexor tendon in one experiment, and with tail tendon in another. Hybridized arrays were then imaged and fluorescence quantitation was made for each dye and each spot.

Genes that were expressed at an at least 2 fold higher level in AT versus flexor tendon included loricrin (Lor; GENBANK®Accession No. NM_{—}0085087), keratin complex 2, basic, gene 17 (Krt-17; GENBANK® Accession No. NM_{—}010668), small prolinerich-like 3 (Sprrl3; GENBANK® Accession No. NM_{—}025984), keratin complex 1, acidic, gene 10 (Krt1-10; GENBANK® Accession No. NM_{—}010660), lymphocyte antigen 6 complex, locus D (Ly6d; GENBANK® Accession No. NM_{—}010742), filaggrin (Flg; GENBANK® Accession No. AF510860), RIKEN cDNA2200001115 gene (2200001I15 Rik; GENBANK® Accession No. NM_{—}026394), myosin, heavy polypeptide 6, cardiac muscle, alpha (Myh6; GENBANK® Accession No. NM_{—}010856), similar to keratinocyteprolin-rich protein (AA589586; GENBANK® Accession No. AK003253), and adipsin (And; GENBANK® Accession No. NM_{—}013459). Genes that were expressed at an at least 2 fold higher level in AT versus tail tendon included filaggrin (Flg; GENBANK® Accession No. AF510860), loricrin (Lor; GENBANK® Accession No. NM_{—}0085087), calmodulin 4 (Calm4; GENBANK® Accession No. NM_{—}020036); hornerin (GENBANK® Accession No. AY027660), similar to keratinocytesproline-rich protein (LOC433619; GENBANK® Accession Nos. XM_{—}904796 and XM_{—}485267), lymphocyte antigen 6 complex, locus D (Ly6D: GENBANK® Accession No. NM_{—}010742), paired like homeodomain transcription factor 1 (Pitxl; GENBANK® Accession No. NM_{—}011097), keratin complex 1, acidic, gene 10 (Krt1-10; GENBANK® Accession No. NM_{—}010660), small prolinerich-like 2 (Sprrl2; GENBANK® Accession No. NM_{—}028625), small prolinerich-like 10 (Sprrl10; GENBANK® Accession No. AK004318), small prolinerich-like 7 (Sprrl7; GENBANK® Accession No. NM_{—}027137), and serine protease inhibitor, Kazal type 5 (Spink5; GENBANK® Accession No. XM_{—}283487).

### Discussion of Examples 1-5

Disclosed herein are the first results of gene array experiments revealing comparisons of differential gene expression in tendon versus a nearest neighbor tissue (muscle), to a treatment with a cytokine thought to be involved in tendon pathology (IL-1β), and to tendon cells in different genetic environments (P2Y₂ knockout and P2Y₁/P2Y₂ double knockout mice). Inspection of the entire gene list for lower fold changes in expression show other candidate genes such as tenomodulin, thought to be a marker for tendon, and titin, thought to be a marker for muscle, that were expressed to an even greater degree in tendon.

### REFERENCES

The references listed below as well as all references cited in the specification, including patents, patent applications, journal articles, and all database entries (e.g., GENBANK®, TIGR, ENSEMBL, and Agilent Accession numbers, including any annotations presented in the databases associated with the disclosed sequences), are incorporated herein by reference to the extent that they supplement, explain, provide a background for, or teach methodology, techniques, and/or compositions employed herein.
Alam et al. 1990 188 Anal Biochem 245-254.
Albert et al. (1992) J Virol 66:5627-5630.
Alexay et al. (1996) The International Society of Optical Engineering 2705/63.
Altschul (1993) 36 J Mol Evol 290-300.
Altschul et al. (1990) 215 J Mol Biol 403-410.
Altschul et al. (1994) 6 Nature Genet 119-129.
Ausubel et al. (2002) Short Protocols in Molecular Biology, Fifth ed. Wiley, New York, N.Y., United States of America.
Ausubel et al. (2003) Current Protocols in Molecular Biology, John Wylie & Sons, Inc., New York, N.Y., United States of America.
Batzer et al. (1991) 19 Nucleic Acid Res 5081.
Bej et al. (1991) Appl Environ Microbiol 57:3529-3534.
Boom et al. (1990) J Clin Microbiol 28:495-503.
Buffone et al. (1991) Clin Chem 37:1945-1949.
Busch et al. (1992) Transfusion 32:420-425.
Cha & Thilly (1993) PCR Methods Appl 3:S18-S29.
Chiodi et al. (1992) J Clin Microbiol 30:255-258.
Cressman et al. (1999) 274 J Biol Chem 26461-26468.
DeRisi et al. (1996) Nat Genet 14:457-460.
Dubiley et al. (1997) Nuc Acids Res 25:2259-2265.
Englert (2000) in Schena, ed., Microarray Biochip Technology, pp. 231-246, Eaton Publishing, Natick, Mass., United States of America.
Fodor et al. (1991) Science 251:767-773.
Fodor et al. (1993) Nature 364:555-556.
Grant 1995 in Molecular Biology and Biotechnology, Meyers (ed.) VCH Publishers, New York, N.Y., United States of America.
Guedon et al. (2000) Anal Chem 72(24):6003-6009.
Hamel et al. (1995) J Clin Microbiol 33:287-291.
Heaton et al. (2001) Proc Natl Acad Sci USA 98(7):3701-3704.
Henikoff & Henikoff (1992) 89 Proc Natl Acad Sci USA 10915-10919.
Hermanson (1990) Bioconjugate Techniques, Academic Press, San Diego, Calif., United States of America.
Herrewegh et al. (1995) J Clin Microbiol 33:684-689.
Izraeli et al. (1991) Nuc Acids Res 19:6051.
Karlin & Altschul (1993) 90 Proc Natl Acad Sci USA 5873-5877.
Karlin et al. (1990) 87 Proc Natl Acad Sci USA 2264-2268.
Kohsaka & Carson (1994) J Clin Lab Anal 8:425-455.
Lanciotti et al. (1992) J Clin Microbiol 30:545-551.
Leon et al., (1999) 104 J Clin Invest 1731-1737.
Linz et al. (1990) J Clin Chem Clin Biochem 28:5-13.
Lisle et al. (2001) BioTechniques 30:1268-1272.
Liu & Hlady (1996) Coll Sur B 8:25-37.
Lockhart et al. (1996) 14 Nat Biotechnol 1675-1680.
Mace et al. (2000) in Schena, ed., Microarray Biochip Technology, pp. 39-64, Eaton Publishing, Natick, Mass., United States of America.
Maier et al. (1994) J Biotechnol 35:191-203.
McCaustland et al. (1991) J Virol Methods 35:331-342.
McGall et al. (1996) 93 Proc Nat Acad Sci USA 13555-13460.
McPherson et al. (1995) PCR 2: A Practical Approach, IRL Press, New York, N.Y., United States of America.
Millar et al., (1995) Anal Biochem 226:325-330.
Natarajan et al. (1994) PCR Methods Appl 3:346-350.
Needleman & Wunsch (1970) 48 J Mol Biol 443-453.
Nelson et al. (2001) Anal Chem 73(1):1-7.
O'Donnell et al. (1997) Anal Chem 69:2438-2443.
Ohtsuka et al. (1985) 260 J Biol Chem 2605-2608.
Paladichuk (1999) The Scientist 13(16):20-23.
PCT International Patent Application Publications WO 93/09668; WO 95/11755; WO 97/14028; WO 99/19515; WO 99/32660; WO 99/63385; WO 01/13120; WO 01/14589; WO 01/23082.
Pearson & Lipman (1988) 85 Proc Natl Acad Sci USA 2444-2448.
Piétu et al. (1996) Genome Res 6:492-503.
Randolph & Waggoner (1995) Nuc Acids Res 25:2923-2929.
Ratner & Castner (1997) in Vickerman, ed., Surface Analysis: The Principal Techniques, John Wiley & Sons, New York, United States of America.
Robertson & Walsh-Weller (1998) Methods Mol Biol 98:121-154.
Rose (2000) in Schena, ed., Microarray Biochip Technology, pp. 19-38, Eaton Publishing, Natick, Mass., United States of America.
Rossolini et al. (1994) 8 Mol Cell Probes 91-98.
Roux (1995) PCR Methods Appl 4:S185-S194.
Rupp et al. (1988) BioTechniques 6:56-60.
Sambrook & Russell (2001) Molecular Cloning: A Laboratory Manual, Cold Spring Harbor Press, Cold Spring Harbor, N.Y.
Sapolsky & Lipshutz (1996) Genomics 33:445-456.
Schena (2000) Microarray Biochip Technology. Eaton Publishing, Natick, Mass., United States of America.
Schena et al. (1995) Science 270:467-470.
Schena et al. (1996) Proc Natl Acad Sci USA 93:10614-10619.
Shalon et al. (1996) Genome Res 6:639-645.
Shoemaker et al. (1996) Nat Genet 14:450-456.
Shriver-Lake (1998) in Cass & Ligler, eds., Immobilized Biomolecules in Analysis, pp. 1-14, Oxford Press, Oxford, United Kingdom.
Silhavy et al. (1984) Experiments with Gene Fusions, Cold Spring Harbor Laboratory, Cold Spring Harbor, N.Y., United States of America.
Smith (1998) The Scientist 12(14):21-24.
Smith & Waterman (1981) 2 Adv Appl Math 482-489.
Smith et al. (1998) Clin Chem 44(9):2054-2056.
Southern (1975) J Mol Biol 98:503-517.
Strain & Chmielewski (2001) BioTechniques 30(6):1286-1291.
Steel et al. (2000) in Schena, ed., Microarray Biochip Technology, pp. 87-118, Eaton Publishing, Natick, Mass., United States of America.
Tanaka et al. (1994) J Gen Virol 75:2691-2698.
Telenius et al. (1992) Genomics 13:718-725.
Theriault et al. (1999) in Schena, ed., DNA Microarrays: A Practical Approach, pp. 101-120, Oxford University Press Inc., New York, N.Y., United States of America.
Tijssen (ed.) (1993) Laboratory Techniques in Biochemistry and Molecular Biology: Hybridization With Nucleic Acid Probes, Part I Theory and Nucleic Acid Preparation, Elsevier Press, New York, N.Y., United States of America.
U.S. Pat. Nos. 4,729,947; 5,143,854; 5,207,880; 5,230,781; 5,346,603; 5,360,523; 5,445,934; 5,534,125; 5,571,388; 5,743,960; 5,800,992; 5,837,832; 5,843,767; 5,846,717; 5,871,918; 5,916,524; 5,965,352; 5,968,745; 5,974,164; 5,985,557; 5,994,069; 6,001,567; 6,017,696; 6,066,457, 6,086,737; 6,090,543; 6,123,819; 6,127,127; 6,162,603; 6,185,561; 6,225,059; 6,229,911; 6,245,508.
Vankerckhoven et al. (1994) J Clin Microbiol 30:750-753.
Vignali (2000) J Immunol Methods 243(1-2):243-255.
Wang et al. (1998) Proc Natl Acad Sci USA 86:9717-9721.
Warrington et al. (2000) in Schena, ed., Microarray Biochip Technology, pp. 119-148, Eaton Publishing, Natick, Mass., United States of America.
Williams (1989) BioTechniques 7:762-769.
Williams et al. (1990) Nuc Acids Res 18(22):6531-6535.
Worley et al. (2000) in Schena, ed., Microarray Biochip Technology, pp. 65-86, Eaton Publishing, Natick, Mass., United States of America.
Yang et al. (1998) Science 282:2244-2246.
Yershov et al. (1996) Proc Natl Acad Sci USA 93:4319-4918.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for detecting connective tissue-specific gene expression in a sample, the method comprising detecting a level of expression in a sample of at least one gene for which expression is connective tissue-specific.

2. The method of claim 1, wherein the connective tissue is selected from the group consisting of muscle and tendon.

3. The method of claim 2, wherein the connective tissue is tendon.

4. The method of claim 1, wherein the at least one gene is selected from the group consisting of those genes listed in Tables 1-4.

5. The method of claim 1, wherein the detecting comprising hybridizing a nucleic acid isolated from the sample to an array comprising the at least one gene.

6. A method for diagnosing a disease of or an injury to a connective tissue in a mammalian subject, the method comprising detecting a level of expression in a biological sample of at least one gene for which an expression level is indicative of disease or injury in a connective tissue.

7. The method of claim 6, wherein the connective tissue is selected from the group consisting of muscle and tendon.

8. The method of claim 7, wherein the connective tissue is tendon.

9. The method of claim 6, wherein the at least one gene is selected from the group consisting of those genes listed in Tables 1-4.

10. The method of claim 9, wherein differential expression of at least one of the genes listed in Tables 1-4 is indicative of a disease or injury to a tendon.

11. The method of claim 6, wherein the detecting comprising hybridizing a nucleic acid isolated from a sample isolated from the mammalian subject to an array comprising the at least one gene.

12. A method for detecting the progression of a disease of or an injury to a connective tissue in a mammalian subject, the method comprising detecting a level of expression in a biological sample of at least one gene for which an expression level is indicative of progression of a disease or injury in a connective tissue.

13. The method of claim 12, wherein the connective tissue is selected from the group consisting of muscle and tendon.

14. The method of claim 13, wherein the connective tissue is tendon.

15. The method of claim 12, wherein the at least one gene is selected from the group consisting of those genes listed in Tables 1-4.

16. The method of claim 15, wherein differential expression of at least one of the genes listed in Tables 1-4 is indicative of progression of a disease of or an injury to a tendon.

17. The method of claim 12, wherein the detecting comprising hybridizing a nucleic acid isolated from a sample isolated from the mammalian subject to an array comprising the at least one gene.

18. A method for monitoring the treatment of a mammalian subject with a disease of or an injury to a connective tissue, the method comprising:
a) providing a treatment to the subject;
b) detecting a level of expression of at least one gene from a cell or biological sample from the subject; and
c) comparing the level of expression detected in step (b) to a level of expression from a cell population comprising normal connective tissue cells, to a level of expression from a cell population comprising diseased or injured connective tissue, or both.

19. The method of claim 18, wherein the connective tissue is selected from the group consisting of muscle and tendon.

20. The method of claim 19, wherein the connective tissue is tendon.

21. The method of claim 18, wherein the at least one gene is selected from the group consisting of those genes listed in Tables 1-4.

22. The method of claim 21, wherein differential expression of at least one of the genes listed in Tables 1-4 is indicative of an effect of the treatment provided on a disease of or an injury to a tendon.

23. The method of claim 18, wherein the detecting comprising hybridizing a nucleic acid isolated from a sample isolated from the mammalian subject to an array comprising the at least one gene.

24. A kit for detecting expression of a gene differentially expressed in a connective tissue, the kit comprising a plurality of reagents that can be used to detect expression levels for at least one gene for which expression is connective tissue-specific.

25. The kit of claim 24, wherein the at least one gene is selected from the group consisting of those genes listed in Tables 1-4.

26. The kit of claim 24, wherein the plurality of reagents comprise at least one oligonucleotide pair that can be used to specifically amplify the at least one gene for which expression is connective tissue-specific.

27. The kit of claim 26, wherein the at least one gene is selected from the group consisting of those genes listed in Tables 1-4.

28. The kit of claim 24, further comprising one or more solid supports comprising one or more oligonucleotides attached thereto that specifically bind to at least one of the genes listed in Tables 1-4.

29. The kit of claim 28, wherein the one or more solid supports comprise an array, a microarray, or combinations thereof.
